SEQUENCE LISTING (28 sequences)

SEQ ID NO: 1 - DNA, 1551 bp, Homo sapiens
atggctggcagtcttacaggtctcctgctcctgcaagctgtctcttgggcttctggggccaggccctgtatccccaaatcctttggatactcatctgtggtgtgtgtttgtaatgccacttattgtgatagctttgacccccccacctttcctgcactgggcaccttttcaaggtatgaatctaccaggtctgggaggaggatggagctgagtatggggcccatccaagcaaaccatactggcactggcttgctgctgacactgcaacctgaacagaagttccagaaagtgaagggctttggaggagccatgactgatgctgctgccctcaatattttggccctgagcccccctgctcagaatctccttttgaaatcatacttctctgaggagggaattggatacaatatcatcagggtgccaatggcctcatgtgactttagtattaggacttacacctatgctgatacccctgatgatttccagctgcataacttctcattgcctgaggaggataccaaattgaagatcccactcattcacagggccctgcaactggctcagagaccagtgtcattgctggcctccccctggacctccccaacttggctcaaaaccaatggggctgtcaatggtaagggctctcttaaggggcagcctggagacatttaccatcagacctgggccaggtattttgtgaagttcctggatgcttatgctgagcacaaattgcaattttgggctgttacagctgagaatgaaccctctgcaggactgctgtctggctatcctttccagtgcctgggctttacccctgagcatcagagggatttcattgccagggacctgggacctactcttgccaatagcacacaccataatgtgaggcttctgatgcttgatgaccagagacttctgctgccacactgggccaaggttgtcctgacagatcctgaggctgccaagtatgttcatgggattgctgtgcactggtatctggacttccttgctccagctaaggccaccctgggagaaacacacaggttgtttcccaatacaatgctttttgcatcagaggcctgtgtgggcagtaaattttgggagcagtctgttaggctggggagctgggatagaggaatgcaatactcccattctatcatcaccaatctgctctaccatgtggtggggtggactgactggaaccttgcccttaaccctgagggtggccccaattgggtcaggaattttgtggatagtcccatcattgtggatatcaccaaggacacattctataagcaaccaatgttctatcacctgggtcactttagtaagtttatccctgaggggtcccagagggtgggactggtggcttcccagaagaatgatctggatgctgtggccctgatgcaccctgatggcagtgctgtggttgttgttctcaatagaagctctaaagatgtgcccttgaccatcaaagatccagctgtgggatttctggaaacaatttcccctggttatagcatccacacttacctttggagaaggcagtga

SEQ ID NO: 2 - DNA, 388 bp, Homo sapiens
attcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggct

SEQ ID NO: 3 - DNA, 208 bp, Bos taurus
gccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcact

SEQ ID NO: 4 - DNA, 5295 bp, Artificial Sequence
  Recombinant Construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagaggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacagatctgaattcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggctgcagctctaaggtaaatataaaatttttaagtgtataatgtgttaaactactgattctaattgtttctctcttttagattccaacctttggaactcaattcagccaccatggctggcagtcttacaggtctcctgctcctgcaagctgtctcttgggcttctggggccaggccctgtatccccaaatcctttggatactcatctgtggtgtgtgtttgtaatgccacttattgtgatagctttgacccccccacctttcctgcactgggcaccttttcaaggtatgaatctaccaggtctgggaggaggatggagctgagtatggggcccatccaagcaaaccatactggcactggcttgctgctgacactgcaacctgaacagaagttccagaaagtgaagggctttggaggagccatgactgatgctgctgccctcaatattttggccctgagcccccctgctcagaatctccttttgaaatcatacttctctgaggagggaattggatacaatatcatcagggtgccaatggcctcatgtgactttagtattaggacttacacctatgctgatacccctgatgatttccagctgcataacttctcattgcctgaggaggataccaaattgaagatcccactcattcacagggccctgcaactggctcagagaccagtgtcattgctggcctccccctggacctccccaacttggctcaaaaccaatggggctgtcaatggtaagggctctcttaaggggcagcctggagacatttaccatcagacctgggccaggtattttgtgaagttcctggatgcttatgctgagcacaaattgcaattttgggctgttacagctgagaatgaaccctctgcaggactgctgtctggctatcctttccagtgcctgggctttacccctgagcatcagagggatttcattgccagggacctgggacctactcttgccaatagcacacaccataatgtgaggcttctgatgcttgatgaccagagacttctgctgccacactgggccaaggttgtcctgacagatcctgaggctgccaagtatgttcatgggattgctgtgcactggtatctggacttccttgctccagctaaggccaccctgggagaaacacacaggttgtttcccaatacaatgctttttgcatcagaggcctgtgtgggcagtaaattttgggagcagtctgttaggctggggagctgggatagaggaatgcaatactcccattctatcatcaccaatctgctctaccatgtggtggggtggactgactggaaccttgcccttaaccctgagggtggccccaattgggtcaggaattttgtggatagtcccatcattgtggatatcaccaaggacacattctataagcaaccaatgttctatcacctgggtcactttagtaagtttatccctgaggggtcccagagggtgggactggtggcttcccagaagaatgatctggatgctgtggccctgatgcaccctgatggcagtgctgtggttgttgttctcaatagaagctctaaagatgtgcccttgaccatcaaagatccagctgtgggatttctggaaacaatttcccctggttatagcatccacacttacctttggagaaggcagtgaaaatgaaggcctgataattgcaccaccaggcctgataggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcactagtccactccctctctgcgcgctcgctcgctcactgaggccgggcgaccaaaggtcgcccgacgcccgggctttgcccgggcggcctcagtgagcgagcgagcgcgcagagagggatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - protein, 497 aa, Homo sapiens
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 6 - protein, 516 aa, Homo sapiens
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 7 - DNA, 1611 bp, Homo sapiens
atggagttttcaagtccttccagagaggaatgtcccaagcctttgagtagggtaagcatcatggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtga

SEQ ID NO: 8 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - DNA, 644 bp, Artificial Sequence
  Recombinant construct
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 10 - DNA, 720 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 11 - DNA, 1611 bp, Artificial sequence
  Human GBA1 coding nucleotide sequence (IDT optimized sequence)
atggagttctcatctccctcacgagaagaatgtccgaaacctctttcaagagtaagcatcatggccggcagcttgaccggtcttttgttgttgcaggccgtgtcctgggcctcaggtgctaggccatgcattcctaaatccttcggctatagtagcgtggtttgcgtctgcaacgccacatactgtgacagtttcgatccacctaccttcccagcgctgggtaccttctcacggtatgaatcaacgcgatcagggcgcagaatggaactttcaatggggccaatccaagctaaccacacgggaacgggtcttctgctgacgctccaaccggaacaaaagttccaaaaggtaaaaggctttggaggtgcgatgactgatgccgcagcactcaacatcctggcgctctcaccgccggcacaaaatttgctgttgaagagttatttctcagaagaagggatcggttacaacatcatacgggtcccgatggcgagctgtgacttttctataagaacatatacctatgcggatacgcccgacgatttccaacttcataattttagtctgcctgaggaagacacaaagttgaagataccgctgatacacagagcattgcagcttgctcaacgaccggtcagcttgcttgccagcccatggacaagtccaacatggcttaagaccaatggcgcggttaatggcaagggatccctgaagggccagccgggagacatctatcatcaaacttgggcgcggtattttgtcaagttcttggacgcctacgctgagcacaaactgcagttctgggccgttaccgccgaaaatgaaccatccgccggactgctttctggctaccctttccaatgtcttggctttacgcctgaacaccaaagagacttcattgctcgggaccttggtccaacgctcgcgaacagtactcatcataatgtacgactcttgatgctcgatgaccagcgactgttgcttccacattgggccaaggtagttctgaccgaccccgaagccgctaaatacgtccacggcattgctgtccattggtaccttgactttttggctcccgcaaaagccactctgggtgaaacacacagactctttccaaacacgatgcttttcgcatcagaagcctgcgtcggaagtaaattttgggaacagtcagtaaggttgggtagttgggatcgcgggatgcaatatagtcatagcattattaccaacttgctttatcacgtcgttgggtggacagattggaacctcgcgttgaatcctgaaggcggccctaattgggtaagaaactttgttgattcacctattatcgtcgacataaccaaggacacattctacaagcaaccgatgttctatcaccttgggcatttcagtaaattcataccagagggcagccagcgcgtcgggttggtagcctctcaaaaaaacgatttggatgcggtcgctctgatgcatcccgacgggagcgcagtagtcgttgtccttaaccgaagctccaaggatgtacccctcacgattaaggaccctgctgtcgggttccttgaaactataagtcccggctatagtattcatacttatctctggagaagacagtga

SEQ ID NO: 12 - DNA, 1611 bp, Artificial sequence
  Human GBA1 coding nucleotide sequence (GenScript optimized sequence)
atggagttttcaagcccctcacgggaagagtgccctaagcccctgtcacgggtctcaattatggccgggagcctgactggcctgctgctgctgcaggccgtgagctgggcatcaggagccaggccttgcatcccaaagtctttcggctacagctccgtggtgtgcgtgtgcaacgccacctattgtgactccttcgatccccctacctttcccgccctgggcacattttctagatacgagtctacacgcagcggccggagaatggagctgagcatgggccctatccaggccaatcacaccggaacaggcctgctgctgaccctgcagccagagcagaagttccagaaggtgaagggctttggaggagcaatgacagacgcagccgccctgaacatcctggccctgtccccacccgcccagaatctgctgctgaagtcctacttctctgaggagggcatcggctataacatcatccgggtgcccatggccagctgcgacttttccatcagaacctacacatatgccgatacccctgacgatttccagctgcacaatttttccctgccagaggaggatacaaagctgaagatccccctgatccaccgggccctgcagctggcacagcggcccgtgagcctgctggccagcccctggacctcccctacatggctgaagaccaacggcgccgtgaatggcaagggctctctgaagggacagccaggcgacatctaccaccagacatgggccagatatttcgtgaagtttctggatgcctacgccgagcacaagctgcagttctgggccgtgaccgcagagaacgagccttctgccggcctgctgagcggctatcccttccagtgcctgggctttacacctgagcaccagcgggactttatcgccagagatctgggcccaaccctggccaactccacacaccacaatgtgaggctgctgatgctggacgatcagcgcctgctgctgcctcactgggccaaggtggtgctgaccgacccagaggccgccaagtacgtgcacggcatcgccgtgcactggtatctggatttcctggcaccagcaaaggccaccctgggagagacacaccggctgttccctaacaccatgctgtttgccagcgaggcctgcgtgggctccaagttttgggagcagtccgtgaggctgggatcttgggacaggggcatgcagtactcccactctatcatcaccaatctgctgtatcacgtggtgggctggacagactggaacctggccctgaatccagagggcggccccaactgggtgagaaatttcgtggatagccccatcatcgtggacatcaccaaggatacattctacaagcagccaatgttttatcacctgggccacttctctaagtttatcccagagggcagccagagggtgggcctggtggccagccagaagaacgacctggatgccgtggccctgatgcaccctgatggctccgccgtggtggtggtgctgaatcgctctagcaaggacgtgcctctgaccatcaaggatccagccgtgggcttcctggagactatttcccccggctattcaattcatacctatctgtggagaaggcagtga

SEQ ID NO: 13 - DNA, 448 bp, homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 14 - protein, 735 aa, Artificial sequence
  AAV TT capsid protein
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - DNA, 145 bp, Artificial sequence
  Flip ITR of AAV2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 16 - DNA, 145 bp, Artificial sequence
  Flop ITR of AAV2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 17 - protein, 449 aa, homo sapiens
MELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 18 - protein, 449 aa, homo sapiens; identical to SEQ ID NO: 17

SEQ ID NO: 19 - DNA, 1611 bp, Artificial sequence
  human GBA1 gene variant
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 20 - DNA, 376 bp, Artificial sequence
  nGUSB promoter #2
attcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagc

SEQ ID NO: 21 - DNA, 1663 bp, artificial sequence
  CAG promoter #2
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 22 - DNA, 400 bp, Artificial sequence
  Human Ubiquitin C (UbC) promoter short version
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggt

SEQ ID NO: 23 - DNA, 1212 bp, Artificial sequence
  Human Ubiquitin C (UbC) promoter long version
ccggaggcgcggcccaaaaccgcggagggcgcccgcggggggaggagtgccgctcgcgacggtgcagtctgcttcccgcgtcgctcgcaggactaggaaggcgggcctgcgagtcctgtcgccgggcgacgagtattctgagccggaatcttggggtcatagtcgtcttcctgtaaaatcctgccctgaacccactgagatcccgtgaccaaaagaaaggtctctcgccttgtccgctccttttcatcagggaagagccgctaagacgcctccctagaggcaccccgccacttgcggctactaatatattcctgcgcggcccacaccgtgtcgatcaaggcagcgtcggccctaaacccagcgccaagaacaaacacctagcgacactagcagtgaaccactcatcgcccgacgacccgaccggccccgaaagcaccggcggcccggcgagccaccctgccttcgcacacctctctggcggttcccgacatcagacccaggcgctcgttccaacgggacttgacccccaacccccctcgcgtcgttttaccgccgacaagggctcagaacttaccttctgcgaacactccgcccgacactccagcaactttgttccaccccccgtaccacccgccgttcttgggttccagaactccggaagcgattacgccctttcgagaataagcccactctacccgaccccgtggtagacccctgggactgcacttcaaacagtgactgacctcttgagccaaacagcagacaacgcccccgccgtcaataccgccacggcaacccgtcacgtgggcatggaaaccctcgcgcgcgggagcagcacagcactgcagtgggcaagacaaccgaatattacgtcccaccccggtggacggccatccacacgccatccgaaaagaggcagcgtcctgcgtcccaagcccggatcccatccgagaggacttagctgtccgcggcctggagaccactcccctccctattcactccgcagtcaaagaaaccagccaaaatacatggatagaagaattcatcgacttcgaggccaaaacttgatacgcgagccccaaccgctcacacaaaacacttcaaaaaatccgtggaaaactttacattagtaaacccagttatacattaaaagtcacaatctgatcatttaacaggcgatttaagaccggcaaaaaccgaaaaaacaatctg

SEQ ID NO: 24 - DNA, 511 bp, Artificial sequence
  Phosphoglycerate kinase 1 (PGK) promoter
gacccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaacagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttgggg

SEQ ID NO: 25 - DNA, 741 bp, Artificial sequence
  CBA/CBh promoter
agatgtactgccaagtaggaaagtcccgtaaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggaactgaaaaaccagaaagttaactggtaagtttagtctttttgtcttttatttcaggtcctggtggtgcaaatcaaagaactgctcctcagtggatgttgcctttacttctaggcctgtacggaagtgttacttctgctctaaaagct

SEQ ID NO: 26 - DNA, 818 bp, Artificial sequence
  CBA/CBh promoter
agatgtactgccaagtaggaaagtcccgtaaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggagtcgctgcgttgccttcgccccgtgccccgctccgcgccgcctcgcgccgcccgccccggctctgactgaccgcgttactcccacaggtgagcgggcgggacggcccttctcctccgggctgtaattagcaagaggtaagggtttaagggatggttggttggtggggtattaatgtttaattacctgttttacaggcctgaaatcacttggttttaggttgg

SEQ ID NO: 27 - DNA, 195 bp, Artificial Sequence
  Nucleotide sequence of the JeT promoter
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 28 - DNA, 510 bp, Artificial Sequence
  Nucleotide sequence of the Human Growth Hormone polyA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

(19) United States
(12) Patent Application Publication
(10) Pub. No.: US 2022/0298528 A1
(43) Pub. Date: Sep. 22, 2022

(54) VIRAL PARTICLES FOR USE IN TREATING SYNUCLEINOPATHIES SUCH AS PARKINSON'S DISEASES BY GENE THERAPY

(71) Applicants: UCB BIOPHARMA SRL, BRUSSELS (BE); FUNDACION PARA LA INVESTIGACION MEDICA APLICADA, Pamplona (ES); CONSORCIO CENTRO DE INVESTIGACIÓN BIOMÉDICA EN RED, Madrid (ES)

(72) Inventors: Gloria GONZALEZ ASEGUINOLAZA, PAMPLONA (ES); José Luis LANCIEGO PEREZ, PAMPLONA (ES); Ralph Michael LINDEN, Slough, Berkshire (GB)

(21) Appl. No.: 17/634,112

(22) PCT Filed: Aug. 6, 2020
(86) PCT No.: PCT/EP2020/072087

§ 371 Date: Feb. 9, 2022

(30) Foreign Application Priority Data
Aug. 12, 2019 (EP) 19382706.0

(51) Int. Cl. C12N 15/86; C12N 9/24; A61K 49/00; A61P 25/16

(52) U.S. Cl. CPC C12N 15/86; C12N 9/2405; C12Y 302/01045; A61K 49/0004; A61P 25/16; C12N 2750/14143; C12N 2750/14145; C12N 2750/14122; A61K 48/00

(57) ABSTRACT

The present disclosure relates to viral particles for use in treating synucleinopathies, particularly sporadic Parkinson Diseases by gene therapy. More specifically, the present invention relates to a viral particle for use in treating synucleinopathy by gene therapy in a subject in need thereof, said viral particle comprising a nucleic acid construct including a transgene encoding a glucocerebrosidase.

### TECHNICAL FIELD

The present disclosure relates to a viral particle comprising a nucleic acid construct comprising a transgene encoding a glucocerebrosidase and its use thereof in treating synucleinopathies, particularly sporadic Parkinson disease by gene therapy.

### BACKGROUND ART

Parkinson's disease (PD) is an unrelenting neurodegenerative disorder characterized by the loss of dopamine-producing neurons in a brain area known as the substantia nigra pars compacta (SNc). As a result of this neuronal loss, there is a progressive decline on brain levels of dopamine. Reduced dopamine levels induce a dysfunction of basal ganglia circuits, leading to the appearance of typical cardinal symptoms of PD (tremor, rigidity, bradykinesia and postural instability). At present, both pharmacological (levodopa and dopamine agonists) and surgical (deep brain stimulation) treatments are available, these treatments showing a high efficacy in alleviating motor symptoms. However, Parkinson disease is a general neurodegenerative disorder with SNc affected first but not limited to it. Existing therapeutic approaches are merely dopamine symptomatic treatment, without any effect in tuning down disease progression.

PD and related synucleinopathies are, by large, sporadic brain disorders (also known as idiopathic disorders; accounting for more than 90-95% of the diagnosed patients). Only in a small fraction of patients, a genetic background has been elucidated (familial cases). Among the genetic mutations that have been implicated in the appearance of these synucleinopathies (LRRK2, SNCA, PARKIN, DJ-1, among others), mutations in the GBA1 gene (located in chromosome 1 and coding for a lysosomal enzyme known as glucocerebrosidase) are by far the most numerous ones, and indeed a direct genetic link between homo- and heterozygous GBA1 mutations and increased incidence of PD and dementia with Lewy bodies (DLB) has been clearly demonstrated by Sidransky and co-workers (Sidransky E, et al. N Engl J Med 2009; 361:1651-1661), with an odds ratio of 5.43 for PD. The association of GBA1 mutations and DLB is even stronger than for PD (odds ratio of 8.28). GBA1 mutations confer a 20- to 30-fold increased risk for the development of PD and DLB, whereas the association between GBA1 mutations and Multiple System Atrophy (MSA) still is more controversial. The presence of such a direct link between GBA1 mutations and synucleinopathies is the strongest argument linking glucocerebrosidase (GCase) deficit with the appearance of PD and DLB. However, in terms of mechanisms engaged in the GCase-alpha-synuclein pathway, how exactly the degradation, aggregation and subcellular processing of alpha-synuclein and GCase activity are coupled together still is an open question in the field with limited experimental evidence.

To date, three main hypotheses linking GCase deficit with alpha-synuclein have been suggested: 1/a gain-of-function by the misfolded GCase (e.g. misfolded because of the GBA1 mutation) results in its direct interaction with alpha-synuclein, ultimately leading to alpha-synuclein aggregation and accumulation; 2/a loss-of-function of GCase (GCase deficiency because of degradation of the misfolded enzyme) leads to accumulation of a substrate (glucocerebroside) that in turn perturbs lipid homeostasis and subsequently affects alpha-synuclein trafficking, processing and clearance. This eventually promotes alpha-synuclein aggregation and facilitates oligomer formation; 3/a bidirectional feedback loop in which GCase deficiency facilitates formation of alpha-synuclein oligomers, these oligomers leading to further decrease in normal GCase activity, which in turn promotes formation of additional alpha-synuclein oligomers.

It has been postulated that 5-10% of PD patients hold a GBA1 mutation. For this subgroup of PD patients, the presence of a GBA1 mutation induces a severe GCase loss-of-function (leaving a residual GCase activity of no more that 15% of normal levels). This GCase loss-of-function triggers alpha-synuclein aggregation (through some unknown mechanisms), ultimately leading to neuronal death. It is also worth noting that for patients with sporadic PD and DLB, it has been postulated that aggregation of alpha-synuclein in itself (e.g. in the absence of GBA1 mutations) may also induce a GCase loss-of-function (Gegg M E, et al., Ann Neurol 2012; 72:455-463; Murphy K E, et al., Brain 2014; 137:834-848; Alcalay R N et al., Brain 2015; 138:2648-2658; Parnetti L, et al., Mov Disord 2014; 29:1019-1027), although the ultimate basis for this association has remained elusive, with very little experimental evidence to date.

In other words, the potential usefulness of enhancing GCase activity in sporadic synucleinopathies still remains to be demonstrated in pre-clinical research for the systemically delivery of recombinant enzyme.

Enhancement of GCase activity in an attempt to reduce alpha-synuclein burden at least in GBA1 mutation-associated patients have been, to some extent, explored. Direct supplementation of recombinant GCase enzyme (GCase replacement therapy) has been a successful treatment in Gaucher disease (Weinreb N J, et al., Am J Med 2002; 113-112-119; Connock M, et al., Health Technol Assess 2006; 10:iii-iv,ix-136). Systemically delivered recombinant GCase has been shown to localize to the lysosome and upregulate enzyme activity. However, the limitation of this approach in the context of neurodegenerative disorders is that GCase may not cross the blood brain barrier in significant concentrations to modify GCase brain activity. An alternative approach is direct intrathecal administration of recombinant GCase (Brady R O, Yang C, Zhuang Z. J Inherit Metab Dis 2013; 36:451-454; LeBowitz J. A Proc Natl Acad Sci USA 2015; 102:14485-14486). However, doubt remains over the ability of intrathecally administered GCase to provide a sufficient concentration gradient to penetrate deeply into neuronal tissues.

Other approaches have focused on glucosylceramide accumulation as a pathogenic mechanism of PD in GBA1 mutation carriers (Sardi S P, et al., Proc Natl Acad Sci USA 2013; 110:3537-3542). GCase substrate inhibition appears to reduce alpha-synuclein levels in synuclein-overexpressing cell lines (Sardi S P, et al., Proc Natl Acad Sci USA 2013; 110:3537-3542). Furthermore, miglustat, a reversible inhibitor of glucosylceramide synthase, has been used in the context of Gaucher disease type III although concerns exist over its efficacy and side effect profile. Two other glucosylceramide synthase inhibitors, eliglustat and venglustat, are under evaluation in clinical trials of Gaucher disease (ClinicalTrials.gov identifier NCT00891202) and PD (ClinicalTrials.gov identifier NCT02906020).

GCase chaperones have also been used to promote the transport of GCase from the ER to the lysosome. Isofagomine was the chaperone tested firstly, although clinical trials of this compound in the context of Gaucher disease were unsuccessful probably due to the fact that this chaperone binds with too high affinity to the active site of GCase, therefore inhibiting enzymatic activity within the lysosome. To circumvent this issue, a number of novel non-inhibitory small molecular chaperones of GCase as potential neuroprotective agents in PD are currently under development (Mazzuli J R, et al. J Neurosci 2016; 36:7693-7706). At present, the most likely small GCase molecular chaperon candidate in PD is ambroxol. In mice, GCase activity increased upon treatment with ambroxol (Migdalska-Richards A, Daly L, Bezard E, Schapira A H. Ann Neurol 2016; 80:766-775), and the same holds true in a non-human primate but only at a higher dose (Migdalska-Richards A, Ko W K D, Li Q et al. Synapse 2017; 256:e21967). Two phase II clinical trials of ambroxol in PD are underway (ClinicalTrials.gov identifiers NCT02941822 and NCT02914366).

Gene therapy with GBA1 has been reported, albeit only in mice. The co-injection of adeno-associated viral vectors coding for GBA1 and mutated alpha-synuclein in rats prevented dopaminergic neurons from neurodegeneration (Rocha E M, et al., Neurobiol Dis 2015; 82:495-503). However, the usefulness of AAV-GBA1 once the synucleinopathy is already in place has not been shown in this study. Furthermore, it has been reported that the use of a blood-brain barrier (BBB) penetrant AAV variant known as AAV9-PHP.B coding for the GBA1 gene in alpha-synuclein transgenic mice resulted in an almost complete clearance of alpha-synuclein aggregates throughout the mice brain (Morabito G, et al., Mol Ther 2017; 25:2727-2742). However, the authors used in this study a transgenic alpha-syn mice lacking the adequate PD-like phenotype. Moreover, AAV9-PHP.B has been shown to only work in specific mice strain and AAV9-PHP-B does not cross the blood-brain barrier in marmosets.

It still remains the need of a gene therapy with GBA1 suitable to treat sporadic Parkinson's Disease in human subject.

In particular, disease-modifying therapies for sporadic PD should be supported by proof-of-principle that the viral-mediated enhancement of GCase activity induces alpha-synuclein aggregates' clearance in dopaminergic neurons of the substantia nigra pars compacta (SNc) both in early and late stages of the disease along with the demonstration that the GCase-driven clearance of aggregated alpha-synuclein is neuroprotective for dopaminergic neurons and impedes the trans-neuronal passage of alpha-synuclein (prion-like spread). In addition, disease-modifying therapies for sporadic PD should also be supported by the attenuation of microglial-driven pro-inflammatory phenomena triggered by alpha-synuclein aggregation.

The present invention provides viral particles for viral-mediated enhancement of GCase activity for use in gene therapy for treating Parkinson's disease in patients at advanced stages of the disease, where a widespread synucleinopathy is present throughout the brain, in particular where engaging the cerebral cortex.

### SUMMARY

The inventors have designed therapeutic strategies that meet the above requirements as demonstrated in the non-human primate models of sporadic Parkinson's disease.

Accordingly, the present invention relates to a viral particle comprising a nucleic acid construct including a transgene encoding a glucocerebrosidase and its use in treating synucleinopathy by gene therapy in a subject in need thereof.

In one embodiment, said transgene comprises a) a nucleotide sequence selected from the group consisting of SEQ ID NO: 1, 7, 11, 12 and 19 or b) a nucleotide sequence encoding human glucocerebrosidase wherein human glucocerebrosidase comprises SEQ ID NO: 5, 6, 8, 17 or 18. In specific embodiments, said nucleic acid construct further comprises a promoter operably-linked to the transgene encoding glucocerebrosidase and wherein said promoter allows the expression of said transgene at least in neuronal and microglial cells of the substantia nigra pars compacta (SNc); and preferably also in neuronal cells of other brain areas, including at least the substantia nigra pars compacta, cerebral cortex, amygdala, and caudal intralaminar nuclei of the thalamus. Typically, said nucleic acid construct may comprise a transgene encoding a glucocerebrosidase under the control of an ubiquitous promoter, for example the GusB promoter, notably a promoter comprising or consisting of SEQ ID NO: 2 or 20, the JeT promoter comprising or consisting of SEQ ID NO: 27, the CAG promoter comprising or consisting of SEQ ID NO: 9 or 21 or human synapsin 1 promoter (hSyn) comprising or consisting of SEQ ID NO: 13.

In specific embodiments, said viral particle includes capsid protein selected among viral particles that simultaneously target at least neurons and microglial cells. More specifically, said viral particle may be selected among viral particles that simultaneously target at least dopaminergic neurons and microglial cells in the substantia nigra pars compacta.

In certain embodiments, said viral particle is selected among rAAV particles, preferably including capsid proteins selected from the group consisting of: AAV2, AAV5, AAV9, AAV-MNM004, AAV-MNM008, and AAV TT serotypes.

In a more specific embodiment, said viral particle includes AAV TT capsid protein, preferably which comprises a sequence of SEQ ID NO: 14 or sequence having at least 95%, 96%, 97%, 98% preferably 98.5%, more preferably 99 or 99.5% identity with SEQ ID NO: 14. In one preferred embodiment, the viral particle comprises a nucleic acid construct comprising:
a) a transgene encoding a human glucocerebrosidase; wherein said transgene comprises SEQ ID NO: 19 or a sequence encoding human glucocerebrosidase, wherein human glucocerebrosidase comprises SEQ ID NO: 5 or 8;
b) a promoter operably-linked to said transgene and wherein said promoter preferably allows the expression of said transgene at least in neuronal and microglial cells of the substantia nigra pars compacta (SNc); wherein said promoter is preferably CAG promoter comprising SEQ ID NO: 9 or 21, or GusB promoter comprising SEQ ID NO:2 or 20, or the JeT promoter comprising SEQ ID NO: 27 or hSyn promoter comprising SEQ ID NO: 13;
c) a polyadenylation signal sequence, preferably a polyadenylation signal sequence comprising SEQ ID NO: 28 or 3, preferably SEQ ID NO: 28;
   wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16. In certain embodiments, said viral particle comprises viral capsid protein selected among viral variant serotypes with retrograde transport (AAVretro).

Typically, said AAVretro may be able to retrogradely disseminate in the cerebral cortex, preferably at least to the substantia nigra pars compacta and cerebral cortex after parenchymal injection in the caudate or putamen nuclei of a non-human primate as determined in an in vivo dissemination assay. Advantageously, AAVretro injected in the caudate-putamen nuclei of a non-human primate may be able to retrogradely disseminate also to other brain areas innervating the caudate-putamen nuclei, including at least substantia nigra pars compacta, cerebral cortex, amygdala, and caudal intralaminar nuclei of the thalamus. In another aspect, the present disclosure relates to an in vivo dissemination assay includes the following steps:
a) injecting a test rAAV comprising GFP (green-fluorescent protein) encoding transgene (rAAV-GFP) by intraparenchymal injection of said rAAV-GFP into the post-commissural putamen of a non-human primate, and,
b) counting the number of GFP-expressing neurons in the cerebral cortex, preferably in brain areas innervating the caudate putamen nuclei, one month post injection, more particularly at least in the substantia nigra pars compacta, the cerebral cortex, the amygdala, and the caudal intralaminar nuclei of the thalamus.

In other embodiments, said in vivo dissemination assay further comprises a step c) of comparing the percentage of labeled neurons in the cerebral cortex, preferably in the brain areas innervating the caudate putamen nuclei with a control experiment performed with AAV-TT-GFP.

In certain embodiments, the viral particle according to the present disclosure, is advantageously selected among AAVretro particles which are able to disseminate in the cerebral cortex, preferably to at least to the substantia nigra pars compacta and the cerebral cortex, to at least the same level as AAV-TT as determined in an in vivo dissemination assay as described above.

In specific embodiments, said AAVretro capsid protein is selected among the following variant serotypes: AAV-MNM004, AAV-MNM008 and AAV-TT.

In a more specific embodiment, said AAV retro particle includes AAV TT serotype capsid protein, preferably which comprises a sequence of SEQ ID NO: 14 or sequence having at least 95%, 96%, 97%, 98%, preferably 98.5%, more preferably 99 or 99.5% identity with SEQ ID NO: 14.

In specific embodiments, said nucleic acid construct further comprises a polyadenylation signal sequence, notably a polyadenylation signal sequence of sequence SEQ ID NO: 3.

In specific embodiments, said nucleic acid construct is comprised in a viral vector which further comprises a 5′ITR and a 3′ITR sequences, preferably a 5′ITR and a 3′ITR sequences of an adeno-associated virus, more preferably a 5′ITR and a 3′ITR sequences from the AAV2 serotype which comprise or consist of sequence SEQ ID NO: 15 and/or 16 or a sequence having at least 80% or at least 90% of identity with SEQ ID NO: 15 and/or 16.

In specific embodiments, said nucleic acid construct comprises a nucleic acid sequence of SEQ ID NO: 4 or a nucleic acid sequence having at least 80% or at least 90% of identity with SEQ ID NO: 4.

In particular embodiments, said nucleic acid construct comprises a coding sequence of human glucocerebrosidase under the control of a promoter, allowing expression of said human glucocerebrosidase in at least both dopaminergic neurons and microglial cells, and said viral particle is selected among viral particles that targets at least dopaminergic neurons and microglial cells of the substantia nigra pars compacta, typically AAV particles including capsid proteins selected from the group consisting of AAV2, AAV5, AAV9, AAV-MNM004, AAV-MNM008, and AAV TT serotypes.

In another aspect, the disclosure relates to the use of a viral particle as described above in therapy, preferably in treating synucleinopathy by gene therapy in a subject in need thereof.

In specific embodiments, said synucleinopathy is a human sporadic synucleinopathy. In particular embodiments, said synucleinopathy is not associated to at least a mutation in a gene selected from the group consisting of LRRK2, SNCA, VPS35, GCH1, ATXN2, DNAJC13, TMEM230, GIGYF2, HTRA2, RIC3, EIF4G1, UCHL1, CHCHD2, GBA1, PRKN, PINK1, DJ1, ATP13A2, PLA2G6, FBXO7, DNAJC6, SYNJ1, SPG11, VPS13C, PODXL, PTRHD1, RAB39B, DNAJC13, TMEM230, GIGYF2, HTRA2, RIC3, EIF4G1, UCHL1, and CHCHD2.

Preferably, said synucleinopathy is a Parkinson's disease, typically sporadic Parkinson's disease.

In specific embodiments, when using AAVretro viral particles according to the present method of treatment, said subject to be treated is selected among patients with advanced stages of synucleinopathy, typically, at least H-Y stage 3 of Parkinson disease.

Said viral vector may preferably be administered to said subject by intraparenchymal administration, more preferably to the brain area of the substantia nigra pars compacta and/or the caudate putamen nuclei.

In another aspect, the disclosure also relates to the use of a viral particle as described above in treating neuronopathic Gaucher disease.

### BRIEF DESCRIPTION OF THE DRAWINGS

FIG. 1 is the amino acid sequence alignment of AAV-TT capsid protein sequence with AAV-2.

FIG. 2 is the amino acid sequence alignment of AAV-TT capsid protein sequence with AAV-9.

FIG. 3 is a cartoon summarizing the experimental approach carried out in mice.

FIG. 4 is a picture of western blots showing protein levels for GCase (left panel) and alpha-synuclein (right panel) in the left and right SNc having received rAAV9-null (without transgene) and rAAV9-GBA1 particles respectively. Obtained results showed that GCase expression resulted in alpha-synuclein reduction.

FIG. 5 is a histogram showing unbiased stereological estimation of the neuronal density of TH+ neurons in the SNc. Obtained results showed that alpha-synuclein clearance upon AAV-GBA1 mediated enhancement of GCase enzymatic activity induced a marked neuroprotective effect on dopaminergic neurons.

FIG. 6 is a cartoon summarizing the experimental approach carried out in non-human primates (NHPs) with rAAV2/9-GBA1 (also referred in the first example as rAAV9) (injected into the left SNc).

FIG. 7 shows coronal brain sections taken at the level of the post-commissural putamen and caudate nuclei as representative images of the obtained microPET scans regarding the NHPs being injected with rAAV9-GBA1 into the left SNc.

FIG. 8 is a histogram showing the results obtained after the unbiased stereological estimation of the density of TH+ neurons in the SNc of 4 NHPs injected with rAAV9-GBA1 into the left SNc. Quantification has been performed in one animal. 38.1% of SNc neurons died after 3 months from rAAV9-SynA53T delivery, contrasting with 14.9% of neuronal death observed in the left SNc (i.e. the one treated with rAAV9-GBA1).

FIG. 9: Cartoon summarizing the conducted experimental plan.

FIG. 10: Photomicrographs taken from coronal sections of the mice brain through the striatum and cerebral cortex showing the immunohistochemical detection of phosphorylated alpha-synuclein. A clear reduction in alpha-synuclein burden at the level of the cerebral cortex is seen in the cerebral cortex located ipsilaterally to the striatum injected with AAV2-retro-GBA1 (GusB promoter). A clear difference was observed when compared to the right cerebral cortex, i.e. the brain side treated firstly with AAV2-retro-SynA53T and later on with AAV2-retro-null (GusB promoter). Obtained data support the use of retrogradely-spreading viral vectors coding for the GBA1 gene for the treatment of disseminated synucleinopathies.

FIG. 11 is a cartoon summarizing the experimental approach carried out in non-human primates (NHPs) with rAAV-TT-GBA1 (injected into the left post-commissural putamen).

FIG. 12 shows coronal brain sections taken at the level of the post-commissural putamen and caudate nuclei as representative images of the obtained microPET scans regarding the NHPs being injected with rAAV-TT-GBA1 into the post-commissural putamen (top panel). On average, a 24.44% increase in 11C-DTBZ binding potential was observed in the left post-commissural putamen (e.g. the one injected with rAAV-TT-GBA1) (bottom panel).

FIG. 13 is a histogram showing the results obtained after the automated counting of the number of TH+ neurons in the SNc of 4 NHPs injected with rAAV-TT-GBA1 into the left post-commissural putamen. Quantification has been performed in 4 NHPs, showing that the total number of TH+ neurons in the left SNc was 22.3% higher than the number of TH+ neurons observed in the right SNc (e.g. the one not treated with the intraputaminal delivery of rAAV-TT-GBA1). * represents statistical significance p<0.05.

FIG. 14 is a histogram showing the results obtained after the automatic measuring of the optical density (OD) for TH stain into the left and right post-commissural putamen of 4 NHPs injected with rAAV-TT-GBA1 into the left post-commissural putamen. Quantification showed that the mean OD was 27.42% lower in the right vs. left post-commissural putamen (the latter being the one treated with the intraputaminal delivery of rAAV-TT-GBA1). *** represents statistical significance p<0.001.

FIG. 15 is a histogram showing the results obtained after the automated counting of the number of alpha-synuclein-expressing neurons in the SNc of 4 NHPs injected with rAAV-TT-GBA1 into the left post-commissural putamen. Quantification has been performed in 4 NHPs, showing that the total number of alpha-synuclein-expressing neurons in the left SNc was 38.3% lower than the number of alpha-synuclein-positive neurons observed in the right SNc (e.g. the one not treated with the intraputaminal delivery of rAAV-TT-GBA1). * represents statistical significance p<0.05, ** represents statistical significance p<0.001, and *** represents statistical significance p<0.001. ns:no statistical significance.

FIG. 16 is a histogram showing the differences in comparison between TH+(black bars) and a-Syn+ neurons (white bars) in the left vs. the right SNc in the animals tested. Percentages indicate the percentage of a-Syn+ neurons in the TH+ neurons. The percentage of alpha-synuclein-expressing neurons are consistently lower in the left SNc (e.g. the one located ipsilaterally to the post-commissural putamen being injected with AAV-TT-GBA1) than in the right SNc (untreated side). This shows that not only the neuronal survival in the left SNc is higher, but also that fewer of those surviving TH+ neurons are a-Syn+.

FIG. 17: Sagittal Rx plates showing the injection sites for all AAVs during ventriculography-assisted stereotaxic surgery.

FIG. 18: Representative photomicrographs showing the injection sites for all AAVs.

FIG. 19: Cartoons illustrating the injection sites for all animals, together with the precise location of GFP+ neurons (A: M295 and 296, B: 297 and 298).

FIG. 20: Biodistribution, and estimated intensities of GFP+ neurons in animals M295 (A) and M296 (B) (injected with AAV-TT-GFP). Small-sized dots (labeled as “low”) represent between 1 to 200 GFP+ cells; medium-sized dots (labeled as “moderate”) represent between 201 to 400 GFP+ cells, and large-sized dots (labeled as “high” represent more than 401 GFP+ cells.

FIG. 21: Biodistribution, and estimated intensities of GFP+ neurons in animals M297 (A) and M298 (B) (injected with AAV-9-GFP). Small-sized dots (labeled as “low”) represent between 1 to 200 GFP+ cells; medium-sized dots (labeled as “moderate”) represent between 201 to 400 GFP+ cells, and large-sized dots (labeled as “high” represent more than 401 GFP+ cells.

FIG. 22: Quantification. Histograms showing the total number of GFP+ neurons for all animals.

FIG. 23: Quantification. Histograms showing the number of GFP+ neurons for all animals across a number of regions of interest. Abbreviations: Anterior cingulate gyms (AcGg), Superior frontal gyrus (SFG), Precentral (PrG), Postcentral gyrus (PoG), Insular gyrus (Ing), Centromedian-parafascicular complex (CM-Pf), Substantia nigra pars compacta (SNc).

FIG. 24: Quantification. Graphs showing the rostrocaudal distribution of GFP+ neurons for all animals across a number of regions of interest of the left hemisphere. Abbreviations: Anterior cingulate gyms (AcGg), Superior frontal gyms (SFG), Precentral (PrG), Postcentral gyms (PoG), Insular gyrus (Ing), Centromedian-parafascicular complex (CM-Pf), Substantia nigra pars compacta (SNc).

FIG. 25: Quantification. Graphs showing the rostrocaudal distribution of GFP+ neurons for all animals across a number of regions of interest of the right hemisphere. Abbreviations: Anterior cingulate gyms (AcGg), Superior frontal gyms (SFG), Precentral (PrG), Postcentral gyms (PoG), Substantia nigra pars compacta (SNc).

FIG. 26: Workplan of conducted experiments.

FIG. 27: MicroPET scans with 11C-DTBZ performed at baseline, and 4-, 8- and 12-weeks post-injection of AAV9-SynA53T. Values for radiotracer binding potential were calculated through regions of interest comprising the entire rostrocaudal extent of the post-commissural putamen (typically engaging between 5 and 9 different sections).

FIG. 28: Histograms showing mean OD values for both animal groups as well as for each animal when considered individually. Representative photomicrographs taken at the level of the post-commissural putamen were also included.

FIG. 29: Graphs illustrate the observed changes in OD through the rostrocaudal extent of the post-commissural putamen (from 0 more rostral to 12 more caudal). OD differences by comparing left vs. right putamen (e.g. side treated with GBA1-coding vectors vs. untreated side, respectively) were drawn by areas shaded (corresponding to animals injected with either AAV-tt-GBA1 or with AAV9-GBA1).

FIG. 30: Histograms showing TH+ cell numbers for both animal groups as well as for each animal when considered individually. Mean values for left vs. right substantia nigra are statistically significant when considering each animal group as a whole. Representative photomicrographs taken at the level of the left and right substantia nigra were also included to illustrate the Aiforia®-conducted analyses in a more visual way.

FIG. 31: Graphs illustrate the observed changes in TH+ cell numbers through the rostrocaudal extent of the substantia nigra (from 0 more rostral to 14 more caudal). Observed differences when comparing left vs. right substantia nigra (e.g. side ipsilateral to the injections with GBA1-coding vectors vs. non-injected side, respectively) were drawn by areas shaded (corresponding to animals injected with either AAV-TT-GBA1 or with AAV9-GBA1).

FIG. 32: Histograms showing α-syn+ cell numbers for both animal groups as well as for each animal when considered individually. Mean values for left vs. right substantia nigra are statistically significant when considering each animal group as a whole. At the individual level, animals M280, M282 and M287 (all treated with AAV-TT-GBA1) also reached statistical significance, whereby animal M283 did not. Regarding animals injected with AAV9-GBA1, statistical significance was only observed in animal M286. Representative photomicrographs taken at the level of the left and right substantia nigra were also included to illustrate the Aiforia®-conducted analyses in a more visual way.

FIG. 33: Graphs illustrate the observed changes in α-syn+ cell numbers through the rostrocaudal extent of the substantia nigra (from 0 more rostral to 12 more caudal). Observed differences when comparing left vs. right substantia nigra (e.g. side ipsilateral to the injections with GBA1-coding vectors vs. non-injected side, respectively) were drawn by areas shaded (corresponding to animals injected with either AAV-TT-GBA1 or with AAV9-GBA1).

FIG. 34: Histogram illustrates the observed ratios between TH+ cells and α-syn+ cells (respectively TH and SYN).

### DETAILED DESCRIPTION

The inventors have identified new therapeutic strategies to treat synucleinopathies by gene therapy, and more specifically Parkinson's diseases, in particular sporadic Parkinson's disease.

The disclosure therefore relates to a viral particle, and its use in treating synucleinopathy, such as Parkinson's disease or neuronopathic Gaucher disease by gene therapy in a subject in need thereof, said viral particle comprising a viral vector or a nucleic acid construct which includes a transgene encoding a glucocerebrosidase.

As used herein, the term “viral particle” relates to an infectious and typically replication-defective virus particle comprising (i) a viral vector packaged within (ii) a capsid and optionally, (iii) a lipidic envelope surrounding the capsid.

The term “viral vector” typically refers to the nucleic acid part of the viral particle as disclosed herein, which is packaged in a capsid.

Said viral vector thus typically comprises at least (i) a nucleic acid construct including a transgene and suitable nucleic acid elements for its expression in a host treated by gene therapy, and (ii) all or a portion of a viral genome, for example the inverted terminal repeats of a viral genome.

As used herein, the term “nucleic acid construct” refers to a non-naturally occurring nucleic acid resulting from the use of recombinant DNA technology. Especially, a nucleic acid construct is a nucleic acid molecule which has been modified to contain segments of nucleic acid sequences, which are combined or juxtaposed in a manner which would not otherwise exist in nature.

As used herein, the term “transgene” refers to nucleic acid molecule (or nucleic acid in short), DNA or cDNA encoding a gene product for use as the active principle in gene therapy. The gene product may be an RNA, peptide or a protein.

The terms “nucleic acid” and “polynucleotide” or “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholines and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-O-allyl analogs and 2′-O-methylribonucleotide methylphosphonates which may be used in a nucleotide of the invention.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

As used here, the term “comprising” does not exclude other elements. For the purposes of the present disclosure, the term “consisting of” is considered to be a preferred embodiment of the term “comprising of”.

As used here, the term “notably”, “typically” or “particularly” are used interchangeably to refer to one alternative among several embodiments and the term “preferably” refers to a preferred embodiment.

As used here SNc is the acronym of substantia nigra pars compacta (SNc).

The Nucleic Acid Constructs of the Present Disclosure

The nucleic acid construct according to the present disclosure include a transgene and at least suitable nucleic acid elements for its expression in said host treated by gene therapy with the viral vector of the disclosure.

For example, said nucleic acid construct comprises a transgene consisting of the coding sequence of glucocerebrosidase and one or more control sequence required for expression of said coding sequence in the relevant cell types or tissue. Generally, the nucleic acid construct comprises a coding sequence and regulatory sequences preceding (5′ non-coding sequences) and following (3′ non-coding sequences) the coding sequence that are required for expression of the selected gene product. Thus, in specific embodiments, said nucleic acid construct comprises at least (i) a transgene encoding a glucocerebrosidase under the control of (ii) a promoter and (iii) a 3′ untranslated region that usually contains a polyadenylation site and/or transcription terminator. The nucleic acid construct may also comprise additional regulatory elements such as, for example, enhancer sequences, introns, microRNA targeted sequence, a polylinker sequence facilitating the insertion of a DNA fragment within a vector and/or splicing signal sequences.

The specific nucleic acid constructs comprise a transgene comprising a nucleotide sequence selected from the group consisting of SEQ ID NO: 1, 7, 11, 12 and 19 or a portion of a nucleic acid sequence selected from the group consisting of SEQ ID NO: 1, 7, 11, 12 and 19 as disclosed hereafter and vectors or particles comprising such specific nucleic acid constructs are also part of the present disclosure.

The Trans Gene Encoding Glucocerebrosidase

In particular, the nucleic acid construct according to the present disclosure comprises a transgene encoding glucocerebrosidase, preferably encoding human glucocerebrosidase comprising SEQ ID NO: 5, 6, 8, 17 or 18, preferably encoding human glucocerebrosidase comprising SEQ ID NO: 5, 6 or 8 (also known as isoform 1).

As used herein, the term “glucocerebrosidase” refers to β-Glucocerebrosidase (also called acid β-glucosidase, D-glucosyl-N-acylsphingosine glucohydrolase, or GCase), an enzyme with glucosylceramidase activity (EC 3.2.1.45) that is needed to cleave, by hydrolysis, the beta-glucosidic linkage of the chemical glucocerebroside, an intermediate in glycolipid metabolism that is abundant in cell membranes (particularly skin cells). The term “glucocerebrosidase” refers to the enzyme and any additional co-translation or post-translational modifications.

Human glucocerebrosidase is naturally encoded by GBA1 gene in human that generated five alternatively spliced mRNAs which encode three distinct isoforms of glucocerebrosidase (isoform 1 (SEQ ID NO: 5), isoform 2 (SEQ ID NO: 17) and isoform 3 (SEQ ID NO: 18)). As used herein, the term “glucocerebrosidase” refers to the three isoforms of glucocerebrosidase. Nucleotide sequence corresponding to the coding sequence portion CDS of human GBA1 mRNA isoform 1 (GeneBank ref. M19285.1:123-1733) is represented by SEQ ID NO: 7.

In specific embodiments, said nucleic acid construct comprises all or a portion (at least 1000, 1100, 1500, 2000, 2500 or at least 1500 nucleotides) of a coding nucleic acid sequence having at least 70%, 80%, 90%; 95%, 99% or 100% identity to the coding sequence of a naturally-occurring or recombinant glucocerebrosidase. Naturally occurring glucocerebrosidases include human, primate, murine or other mammalian known glucocerebrosidases, typically human glucocerebrosidase of SEQ ID NO: 5, 17 or 18.

Examples of recombinant glucocerebrosidase include imiglucerase (Cerezyme), velaglucerase (Vpriv) and taliglucerase (Elelyso).

In a preferred embodiment, said nucleic acid construct comprises a transgene encoding human glucocerebrosidase, wherein said human glucocerebrosidase comprises SEQ ID NO: 5, 6, 8, 17 or 18, for example, a nucleotide sequence as represented by a sequence selected from the group consisting of: SEQ ID NO: 1, 7, 11, 12 and 19, or a variant transgene consisting of a nucleotide sequence having at least 75%, at least 80% or at least 90%, at least 95% or at least 99% identity to a sequence selected from the group consisting of: SEQ ID NO: 1, 7, 11, 12 and 19. Preferably, said transgene includes a portion of a nucleotide sequence selected from the group consisting of: SEQ ID NO: 1, 7, 11, 12 and 19, e.g. the optimized sequence SEQ ID NO: 1, region 58 to 1551 of SEQ ID NO: 7 or 19, region 58 to 1611 of SEQ ID NO: 7 or 19, region 118 to 1611 of SEQ ID NO: 7 or 19. In one embodiment, said variant transgene encoding a portion of SEQ ID NO: 5, 6, 8, 17 or 18 or consisting of a nucleotide sequence having at least 75%, at least 80% or at least 90%, at least 95% or at least 99% identity to a sequence selected from the group consisting of: SEQ ID NO: 1, 7, 11, 12 and 19 that has substantially the same glucocerebrosidase activity as human glucocerebrosidase. In particular, a variant nucleic acid construct encodes a truncated glucocerebrosidase where one or more of the amino acid residues have been deleted.

As used herein, the term “sequence identity” or “identity” refers to the number of matches (identical nucleic acid or amino acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970, J Mol Biol.; 48(3):443-53) which aligns the sequences optimally over the entire length, while sequences of substantially different lengths are preferably aligned using a local alignment algorithm (e.g. Smith and Waterman algorithm (Smith and Waterman, 1981, J Theor Biol.; 91(2):379-80) or Altschul algorithm (Altschul S F et al., 1997, Nucleic Acids Res.; 25(17):3389-402.; Altschul S F et al., 2005, Bioinformatics.; 21(8):1451-6). Alignment for purposes of determining percent nucleic acid or amino acid sequence identity can be achieved in various ways that are within the skill in the art, for instance, using publicly available computer software available on internet web sites such as http://blast.ncbi.nlm.nih.gov/or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid or amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The Promoter for Use with the Nucleic Acid Constructs of the Disclosure

In one embodiment, the nucleic acid construct comprises a promoter. Said promoter initiates transgene expression upon introduction into a host cell.

As used herein, the term “promoter” refers to a regulatory element that directs the transcription of a nucleic acid to which it is operably linked. A promoter can regulate both rate and efficiency of transcription of an operably linked nucleic acid. A promoter may also be operably linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is located near the transcription start site of the gene or coding sequence to which is operably linked, on the same strand and upstream of the DNA sequence (towards the 5′ region of the sense strand). A promoter can be about 100-1000 base pairs long. Positions in a promoter are designated relative to the transcriptional start site for a particular gene (i.e., positions upstream are negative numbers counting back from −1, for example −100 is a position 100 base pairs upstream).

As used herein, the term “operably linked” refers to a linkage of polynucleotide (or polypeptide) elements in a functional relationship. A nucleic acid is “operably linked” when it is placed into a functional relationship with another nucleic acid sequence. For instance, a promoter or transcription regulatory sequence is operably linked to a coding sequence if it affects the transcription of the coding sequence. Operably linked means that the DNA sequences being linked are typically contiguous; where it is necessary to join two protein encoding regions, they are contiguous and in reading frame.

In a particular embodiment, the nucleic acid construct of the disclosure further comprises a promoter operably-linked to the transgene encoding glucocerebrosidase and wherein said promoter directs the expression of said transgene at least in dopaminergic neurons and microglial cells of the substantia nigra pars compacta (SNc), and preferably also in neuronal cells of other brain areas, including at least the substantia nigra pars compacta, cerebral cortex, amygdala, and caudal intralaminar nuclei of the thalamus.

Typically, such promoter may be tissue or cell type specific promoter, or an organ-specific promoter, or a promoter specific to multiple organs or a systemic or ubiquitous promoter.

As used herein, the term “ubiquitous promoter” more specifically relates to a promoter that is active in a variety of distinct cells or tissues of the brain, for example in both the neurons and glial cells, more specifically at least the dopaminergic neurons and microglial cells of the substantia nigra pars compacta, and preferably also in neuronal cells of other brain areas, including at least the substantia nigra pars compacta, cerebral cortex, amygdala, and caudal intralaminar nuclei of the thalamus.

Examples of promoter suitable for expression of the transgene in at least neuronal and microglial cells of the substantia nigra compacta include without limitation CMV promoter (Kaplitt 1994, Nat. Genet. 8:148-154), SV40 promoter (Hamer 1979, Cell 17:725-735), chicken beta actin (CBA) promoter (Miyazaki 1989, Gene 79:269-277), the CAG promoter (Niwa 1991, Gene 108:193-199), the b-glucuronidase promoter (GusB) (Shipley 1991, Genetics 10:1009-1018), the Elongation factor 1 alpha promoter (EF1α) (Nakai 1998, Blood 91:4600-4607), the human synapsin 1 gene promoter (hSyn) (Kugler S. et al. Gene Ther. 2003. 10(4):337-47) or the phosphoglycerate kinase 1 promoter (PGK1) (Hannan 1993, Gene 130:233-239).

In a particular embodiment, said ubiquitous promoter can be selected from the group consisting of: human ubiquitin C (UbC) promoter, preferably of SEQ ID NO: 22 or 23, human

Phosphoglycerate Kinase 1 (PGK) promoter, preferably of SEQ ID NO: 24 and human CBA/CBh promoter of SEQ ID NO: 25 or 26.

In one embodiment, the promoter is the GusB gene promoter, comprising or consisting of SEQ ID NO: 2 or 20. In another embodiment, the promoter is the CAG promoter, comprising or consisting of SEQ ID NO: 9 or 21. In another embodiment the promoter is JeT promoter comprising or consisting of SEQ ID NO: 27. In another embodiment, the promoter is hSyn 1 promoter comprising or consisting of SEQ ID: 13.

All these promoter sequences have properties of allowing expression of said transgene in at least neuronal and microglial cells of the substantia nigra pars compacta.

In a preferred embodiment, said nucleic acid construct includes the GusB promoter comprising or consisting of SEQ ID NO: 2 or 20 operably linked to a transgene encoding a glucocerebrosidase, typically a nucleotide sequence selected from the group consisting of SEQ ID NO: 1, 7, 11, 12 and 19. In another embodiment, said nucleic acid construct includes the JeT promoter comprising or consisting of SEQ ID NO: 27 operably linked to a transgene encoding a glucocerebrosidase, typically a nucleotide sequence selected from the group consisting of SEQ ID NO: 1, 7, 11, 12 and 19. In another embodiment, said nucleic acid construct includes the CAG promoter comprising or consisting of SEQ ID NO: 9 or 21 operably linked to a transgene encoding a glucocerebrosidase, typically a nucleotide sequence selected from the group consisting of SEQ ID NO: 1, 7, 11, 12 and 19. In another embodiment, said nucleic acid construct includes the hSyn promoter comprising or consisting of SEQ ID NO: 13 operably linked to a transgene encoding a glucocerebrosidase, typically a nucleotide sequence selected from the group consisting of SEQ ID NO: 1, 7, 11, 12 and 19.

In specific embodiments, the promoter for use in the present disclosure may be a chemical inducible promoter. As used herein, a chemical inducible promoter is a promoter that is regulated by the in vivo administration of a chemical inducer to said subject in need thereof. Examples of suitable chemical inducible promoters include without limitation Tetracycline/Minocycline inducible promoter (Chtarto 2003, Neurosci Lett. 352:155-158) or rapamycin inducible systems (Sanftner 2006, Mol Ther. 13:167-174).

The Polyadenylation Sequence for Use with the Nucleic Acid Constructs of the Disclosure

Each of these nucleic acid construct embodiments may also include a polyadenylation signal sequence; together or not with other optional nucleotide elements. As used herein, the term “polyadenylation signal” or “poly(A) signal” refers to a specific recognition sequence within 3′ untranslated region (3′ UTR) of the gene, which is transcribed into precursor mRNA molecule and guides the termination of the gene transcription. Poly(A) signal acts as a signal for the endonucleolytic cleavage of the newly formed precursor mRNA at its 3′-end, and for the addition to this 3′-end of a RNA stretch consisting only of adenine bases (polyadenylation process; poly(A) tail). Poly(A) tail is important for the nuclear export, translation, and stability of mRNA. In the context of the invention, the polyadenylation signal is a recognition sequence that can direct polyadenylation of mammalian genes and/or viral genes, in mammalian cells.

Poly(A) signals typically consist of a) a consensus sequence AAUAAA, which has been shown to be required for both 3′-end cleavage and polyadenylation of premessenger RNA (pre-mRNA) as well as to promote downstream transcriptional termination, and b) additional elements upstream and downstream of AAUAAA that control the efficiency of utilization of AAUAAA as a poly(A) signal. There is considerable variability in these motifs in mammalian genes.

In one embodiment, optionally in combination with one or more features of the various embodiments described above or below, the polyadenylation signal sequence of the nucleic acid construct of the invention is a polyadenylation signal sequence of a mammalian gene or a viral gene. Suitable polyadenylation signals include, among others, a SV40 early polyadenylation signal, a SV40 late polyadenylation signal, a HSV thymidine kinase polyadenylation signal, a protamine gene polyadenylation signal, an adenovirus 5 EIb polyadenylation signal, a growth hormone polydenylation signal, a PBGD polyadenylation signal, in silico designed polyadenylation signal (synthetic) and the like.

In a particular embodiment, the polyadenylation signal sequence of the nucleic acid construct is a polyadenylation signal sequence based on human or bovine growth hormone gene. In one embodiment, the polyadenylation signal sequence is based on the bovine growth hormone gene and comprises or consist of SEQ ID NO: 3. In a preferred embodiment, the polyadenylation signal sequence is based on the human growth hormone gene and comprises or consist of SEQ ID NO: 28.

In specific embodiments, the nucleic acid construct, preferably for use according to the present disclosure, includes the GusB promoter comprising or consisting of SEQ ID NO: 2 or 20 operably linked to the coding sequence of GBA1 gene selected from the group consisting of SEQ ID NO: 1, 7, 11, 12 and 19 and the polyadenylation signal sequence comprising or consisting of SEQ ID NO: 28 or SEQ ID NO: 3, preferably SEQ ID NO: 28.

In specific embodiments, the nucleic acid construct, preferably for use according to the present disclosure, includes the CAG promoter comprising or consisting of SEQ ID NO: 9 or 21 operably linked to the coding sequence of GBA1 gene selected from the group consisting of SEQ ID NO: 1, 7, 11, 12 and 19 and the polyadenylation signal sequence comprising or consisting of SEQ ID NO: 28 or SEQ ID NO: 3, preferably SEQ ID NO: 28.

In specific embodiments, the nucleic acid construct, preferably for use according to the present disclosure, includes the hSyn 1 promoter comprising or consisting of SEQ ID NO: 13 operably linked to the coding sequence of GBA1 gene selected from the group consisting of SEQ ID NO: 1, 7, 11, 12 and 19 and the polyadenylation signal sequence comprising or consisting of SEQ ID NO: 28 or SEQ ID NO: 3, preferably SEQ ID NO: 28.

In other specific embodiments, the nucleic acid construct, preferably for use according to the present disclosure, includes the JeT promoter comprising or consisting of SEQ ID NO: 27 operably linked to the coding sequence of GBA1 gene selected from the group consisting of SEQ ID NO: 1, 7, 11, 12 and 19 and the polyadenylation signal sequence comprising or consisting of SEQ ID NO: 28 or SEQ ID NO: 3, preferably SEQ ID NO: 28.

In another preferred embodiment, the nucleic acid construct comprises a) a transgene comprising a nucleotide sequence encoding a human glucocerebrosidase; wherein preferably said nucleotide sequence comprises SEQ ID NO: 19 and wherein human glucocerebrosidase comprises SEQ ID NO: 5 or 8; b) a promoter operably-linked to said transgene, wherein said promoter is preferably i. CAG promoter comprising or consisting of SEQ ID NO: 9 or 21, or ii. hSyn promoter comprising or consisting of SEQ ID NO: 13, or iii. GusB promoter comprising or consisting of SEQ ID NO: 2 or 20, or iv. JeT promoter comprising or consisting of SEQ ID NO: 27; and c) a polyadenylation signal sequence, preferably a polyadenylation signal sequence comprising or consisting of SEQ ID NO: 28 or SEQ ID NO: 3, preferably SEQ ID NO: 28.

Viral Vector

Viral vectors of the present disclosure typically comprise at least (i) a nucleic acid construct including a transgene and suitable nucleic acid elements for its expression in said host treated by gene therapy, and (ii) all or a portion of a viral genome, for example at least inverted terminal repeats of a viral genome.

In one embodiment, the viral vector according to the present disclosure comprises a 5′ITR, and a 3′ITR of a virus.

In one embodiment, the viral vector comprises a 5′ITR and a 3′ITR of a virus independently selected from the group consisting of parvoviruses (in particular adeno-associated viruses), adenoviruses, alphaviruses, retroviruses (in particular gamma retroviruses, and lentiviruses), herpesviruses, and SV40; in a preferred embodiment the virus is an adeno-associated virus (AAV), an adenovirus (Ad), or a lentivirus.

In one embodiment, the viral vector comprises a 5′ITR and a 3′ITR of an AAV.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected. The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length.

AAV ITRs in the viral vectors of the invention may have a wild-type nucleotide sequence or may be altered by the insertion, deletion or substitution of one or more nucleotides, typically, no more than 5, 4, 3, 2 or 1 nucleotide insertion, deletion or substitution as compared to known AAV ITRs. The serotype of the inverted terminal repeats (ITRs) of the AAV vector may be selected from any known human or non-human AAV serotype.

In specific embodiments, the viral vector may be carried out by using ITRs of any AAV serotype. Known AAV ITRs include without limitations, AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV.

In one embodiment, the nucleic acid construct described above is comprised in said viral vector which further comprises a 5′ITR and a 3′ITR of an AAV of a serotype AAV2. In a particular embodiment, the viral vector comprises a 5′ITR and 3′ITR of an AAV of a serotype AAV2, preferably of SEQ ID NO: 15 and/or 16 or a sequence having at least 80% or at least 90% of identity with SEQ ID NO: 15 and/or 16.

Hence, in a more specific embodiment, the viral vector of the disclosure, includes a nucleic acid construct including a GusB promoter of SEQ ID NO: 2 or 20, operably linked to the coding sequence of glucocerebrosidase selected from the group consisting of SEQ ID NO: 1, 7, 11, 12 and 19 and said viral vector further included AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16.

In another specific embodiment, the viral vector of the disclosure, includes a nucleic acid construct including promoter selected from the group consisting of a CAG promoter comprising or consisting of SEQ ID NO: 9 or 21 operably linked to the nucleotide sequence of glucocerebrosidase selected from the group consisting of SEQ ID NO: 1, 7, 11, 12 and 19 and said viral vector further includes AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16.

In another specific embodiment, the viral vector of the disclosure, includes a nucleic acid construct including promoter selected from the group consisting comprising or consisting of a hSyn 1 gene promoter of SEQ ID NO: 13 operably linked to the nucleotide sequence of glucocerebrosidase selected from the group consisting of SEQ ID NO:1, 7, 11, 12 and 19 and further including AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16.

In another specific embodiment, the viral vector of the disclosure, includes a nucleic acid construct including promoter selected from the group consisting comprising or consisting of a JeT promoter comprising or consisting of SEQ ID NO: 27 operably linked to the nucleotide sequence of glucocerebrosidase selected from the group consisting of SEQ ID NO:1, 7, 11, 12 and 19 and further including AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16.

In a particular embodiment, the viral vector of the disclosure comprises or consists of SEQ ID NO: 4 or a sequence having at least 80% or at least 90% of identity with SEQ ID NO: 4.

In a preferred embodiment, the viral vector of the disclosure, includes a nucleic acid construct including promoter selected from the group consisting comprising or consisting of a i. GusB promoter comprising or consisting of SEQ ID NO: 2 or 20; or ii. CAG promoter comprising or consisting of SEQ ID NO: 9 or 21; or iii. hSyn 1 gene promoter comprising or consisting of SEQ ID NO: 13; or iv. JeT promoter comprising or consisting of SEQ ID NO: 27, operably linked to the nucleotide sequence of glucocerebrosidase selected from the group consisting of SEQ ID NO:1, 7, 11, 12 and 19 and further including AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16, wherein more preferably 5′ ITR comprises or consists of SEQ ID NO: 15 and 3′ITR comprises or consists of SEQ ID NO: 16; and wherein the viral vector of the disclosure comprises or consists of SEQ ID NO: 4 or a sequence having at least 80% or at least 90% of identity with SEQ ID NO: 4.

On the other hand, the viral vector of the disclosure can be carried out by using synthetic 5′ITR and/or 3′ITR; and also by using a 5′ITR and a 3′ITR which come from viruses of different serotype. All other viral genes required for viral vector replication can be provided in trans within the virus-producing cells (packaging cells) as described below. Therefore, their inclusion in the viral vector is optional.

In one embodiment, the viral vector comprises a 5′ITR and a 3′ITR of a virus.

Viral Particle

The viral vector as disclosed above may be packaged in a capsid formed by the capsid proteins, thereby constituting a viral particle as described in the next section.

In preferred embodiments, the capsid is formed of capsid proteins of adeno-associated virus, hereafter referred as an AAV vector particle.

As used herein, an AAV vector particle comprises at least 5′ITR and 3′ITR of an AAV genome and capsid proteins of adeno-associated virus. The term AAV vector particle encompasses any recombinant AAV vector particle (rAAV) or mutant AAV vector particle obtained by genetic engineering of known rAAV.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

In a particular embodiment, an AAV viral particle according to the disclosure may be prepared by encapsulating the viral vector of an AAV vector/genome derived from a particular AAV serotype on a viral particle formed by natural Cap proteins corresponding to an AAV of the same particular serotype. Nevertheless, several techniques have been developed to modify and improve the structural and functional properties of naturally occurring AAV viral particles (Bunning H et al. J Gene Med 2008; 10: 717-733). Thus, in another embodiment, AAV viral particles according to the disclosure includes the nucleic acid construct including the gene encoding glucocerebrosidase as flanked by ITR(s) of a given AAV serotype packaged, for example, into: a) a viral particle constituted of capsid proteins derived from the same or different AAV serotype [e.g. AAV2 ITRs and AAV9 capsid proteins; AAV2 ITRs and AAV TT capsid proteins or other capsid proteins from AAVretro serotypes such as AAV2-retro, AAVMNM004 or AAVMNM008; etc]; b) a mosaic viral particle constituted of a mixture of capsid proteins from different AAV serotypes or mutants [e.g. AAV2 ITRs with a capsid formed by proteins of two or multiple AAV serotypes]; c) a chimeric viral particle constituted of capsid proteins that have been truncated by domain swapping between different AAV serotypes or variants [e.g. AAV2 ITRs with AAV5 capsid proteins with AAV3 domains]; or d) a targeted viral particle engineered to display selective binding domains, enabling stringent interaction with target cell specific receptors.

AAV-based gene therapy targeting the CNS have already been reviewed in Pignataro D, Sucunza D, Rico A J et al., J Neural Transm 2018; 125:575-589. More specifically, the AAV particles may be selected and/or engineered to target at least neuronal and microglial cells, and in particular at least the dopaminergic neurons and microglial cells in the substantia nigra pars compacta area (SNc) of the brain.

In specific embodiments, examples of AAV serotype of the capsid proteins for use of AAV viral particle according to the present disclosure include AAV2, AAV5, AAV9, AAV2-retro, AAV MNM004, AAV MNM008, and AAV TT. In more preferred embodiments, said AAV serotype of the capsid proteins are selected from AAV9 and AAV TT serotype.

In a particular embodiment, optionally in combination with one or more features of the various embodiments described above or below, the viral particle is a recombinant AAV viral particle comprising a AAV viral vector as described above, preferably including a nucleotide sequence selected from the group consisting of SEQ ID NO: 1, 7, 11, 12 and 19, and comprising capsid proteins of an AAV9 serotype or of an AAV TT serotype, preferably capsid protein of AAV TT serotype which comprises amino acid sequence SEQ ID NO: 14 or an amino acid sequence having at least 95%, 96%, 97%, 98%, preferably 98.5%, more preferably 99% or 99.5% of identity with SEQ ID NO: 14.

In another specific embodiment, the viral particle comprises a nucleic acid construct including a coding sequence of human glucocerebrosidase under the control of a promoter, said promoter allowing expression of said human glucocerebrosidase in at least both dopaminergic neurons and microglial cells, and said viral particle is selected among viral particles that targets at least dopaminergic neurons and microglial cells of the substantia nigra pars compacta, typically AAV particles including capsid proteins selected from the group consisting of AAV2, AAV5, AAV9, AAV2-retro, AAV MNM004, AAV MNM008, or AAV TT serotypes, preferably capsid protein of AAV TT serotype which comprises amino acid sequence SEQ ID NO: 14 or an amino acid sequence having at least 95%, 96%, 97%, 98%, preferably 98.5%, more preferably 99% or 99.5% of identity with SEQ ID NO: 14.

In a more specific embodiment, such recombinant AAV viral particle according to the present disclosure includes capsid proteins of the AAV9, AAV2-retro, AAV MNM004, AAV MNM008 or AAV TT serotype and a AAV viral vector including (i) a nucleic acid construct comprising a promoter selected from the group consisting of: GusB promoter comprising or consisting of SEQ ID NO: 2 or 20, a CAG promoter comprising or consisting of SEQ ID NO: 9 or 21, a Jet promoter comprising or consisting of SEQ ID NO: 27 and hSyn promoter comprising or consisting of SEQ ID NO: 13, operably linked to a nucleotide sequence of glucocerebrosidase selected from the group consisting of SEQ ID NO:1, 7, 11, 12 and 19 and (ii) AAV ITRs, such as 5′ and 3′ ITRs of AAV2 flanking said nucleic acid construct, preferably 5′ and 3′ ITRs of SEQ ID NO: 15 and/or 16 or a sequence having at least 80% or at least 90% of identity with SEQ ID NO: 15 and/or 16.

In a more specific embodiment, such recombinant AAV viral particle according to the present disclosure includes capsid proteins of the AAV TT serotype which comprises amino acid sequence SEQ ID NO: 14 or an amino acid sequence having at least 95%, 96%, 97%, 98%, preferably 98.5%, more preferably 99% or 99.5% of identity with SEQ ID NO: 14 and a AAV viral vector including (i) a nucleic acid construct comprising a GusB promoter comprising or consisting of SEQ ID NO: 2 or 20 operably linked to a nucleotide sequence of glucocerebrosidase selected from the group consisting of SEQ ID NO:1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

In a more specific embodiment, such recombinant AAV viral particle according to the present disclosure includes capsid proteins of the AAV TT serotype which comprises amino acid sequence SEQ ID NO: 14 or an amino acid sequence having at least 95%, 96%, 97%, 98%, preferably 98.5%, more preferably 99% or 99.5% of identity with SEQ ID NO: 14 and a AAV viral vector including (i) a nucleic acid construct comprising a CAG promoter comprising or consisting of SEQ ID NO: 9 or 21 operably linked to a nucleotide sequence of glucocerebrosidase selected from the group consisting of SEQ ID NO:1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

In a more specific embodiment, such recombinant AAV viral particle according to the present disclosure include capsid proteins of the AAV TT serotype which comprises amino acid sequence SEQ ID NO: 14 or an amino acid sequence having at least 95%, 96%, 97%, 98%, preferably 98.5%, more preferably 99% or 99.5% of identity with SEQ ID NO: 14 and a AAV viral vector including (i) a nucleic acid construct comprising hSyn promoter comprising or consisting of SEQ ID NO: 13 operably linked to a nucleotide sequence of glucocerebrosidase selected from the group consisting of SEQ ID NO:1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

In a more specific embodiment, such recombinant AAV viral particle according to the present disclosure include capsid proteins of the AAV TT serotype which comprises amino acid sequence SEQ ID NO: 14 or an amino acid sequence having at least 95%, 96%, 97%, 98%, preferably 98.5%, more preferably 99% or 99.5% of identity with SEQ ID NO: 14 and a AAV viral vector including (i) a nucleic acid construct comprising JeT promoter comprising or consisting of SEQ ID NO: 27 operably linked to a nucleotide sequence of glucocerebrosidase selected from the group consisting of SEQ ID NO: 1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

In one preferred embodiment, the viral particle comprises nucleic acid construct comprising:

a) a transgene encoding a human glucocerebrosidase; wherein said transgene comprises SEQ ID NO: 19 or a sequence encoding human glucocerebrosidase, wherein human glucocerebrosidase comprises SEQ ID NO: 5 or 8;

b) a promoter operably-linked to said transgene; wherein said promoter is preferably CAG promoter comprising or consisting of SEQ ID NO: 9 or 21, or GusB promoter comprising or consisting of SEQ ID NO: 2 or 20, or JeT promoter comprising or consisting of SEQ ID NO: 27 or hSyn promoter comprising or consisting of SEQ ID NO: 13;

c) a polyadenylation signal sequence, preferably a polyadenylation signal sequence comprising or consisting of SEQ ID NO: 28 or SEQ ID NO: 3, preferably SEQ ID NO: 28;

wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16.

The construction of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801. Viral particles with capsid proteins of the serotype AAV TT have also been described in Tordo J, et al., Brain 2018; 141:2014-2031.

Viral Particle with Retrograde Transport

In some embodiments, said viral particle according to the present disclosure is selected among viral variant serotypes with retrograde transport (AAVretro).

Axonal transport (sometimes also called axoplasmic transport or axoplasmic flow) refers to the movement of cellular organelles and proteins from the cell body of a given neuron toward the axon terminal endings (known as anterograde transport). As used herein, the term “retrograde transport” refers to the transport of particles in the opposite direction, i.e. from the axon terminals back to the parent cell bodies. In this regard, neurotropic viruses (rabies viruses being best example) are typically taken up by axon terminals and transported to the neuron's cell body by taking advantage of retrograde transport.

Examples of AAVretro particles includes without limitation capsid protein, preferably capsid protein of AAV2-retro, AAV-TT, AAV-MNM004 and AAV-MNM008, more preferably VP1 capsid protein of AAV2-retro, AAV-TT, AAV-MNM004 and AAV-MNM008.

AAV2-retro capsid protein has been described in WO2017/218842A1.

Other variegated different types of modified viral capsids such as AAV-TT, AAV-MNM004 and AAV-MNM008, have also been designed to transduce neurons innervating the area where the viral vector is delivered through the retrograde spread of the viral vector.

AAV-MNM004 and AAV-MNM008 are described for example in Davidsson et al. Proc. Natl. Acad. Sci. U.S.A. Dec. 9, 2019 doi: 10.1073/pnas.1910061116 and in WO2019/158619.

AAV-TT capsid also named AAV2 true-type capsid is described for example in WO2015/121501. In one embodiment, AAV-TT VP1 capsid protein comprises at least one amino acid substitution with respect to the wild type AAV VP1 capsid protein at a position corresponding to one or more of the following positions in an AAV2 protein sequence (NCBI Reference sequence: YP_680426.1): 125, 151, 162, 312, 457, 492, 499, 533, 546, 548, 585, 588 and/or 593, more particularly, AAV-TT comprises one or more of the following amino acid substitutions with respect to a wild type AAV2 VP1 capsid protein (NCBI Reference sequence: YP_680426.1): V1251, V151A, A1625, T2055, N3125, Q457M, S492A, E499D, F533Y, G546D, E548G, R5855, R588T and/or A5935. In one particular embodiment, AAV-TT comprises four or more mutations with respect to the wild type AAV2 VP1 capsid protein at the positions 457, 492, 499 and 533.

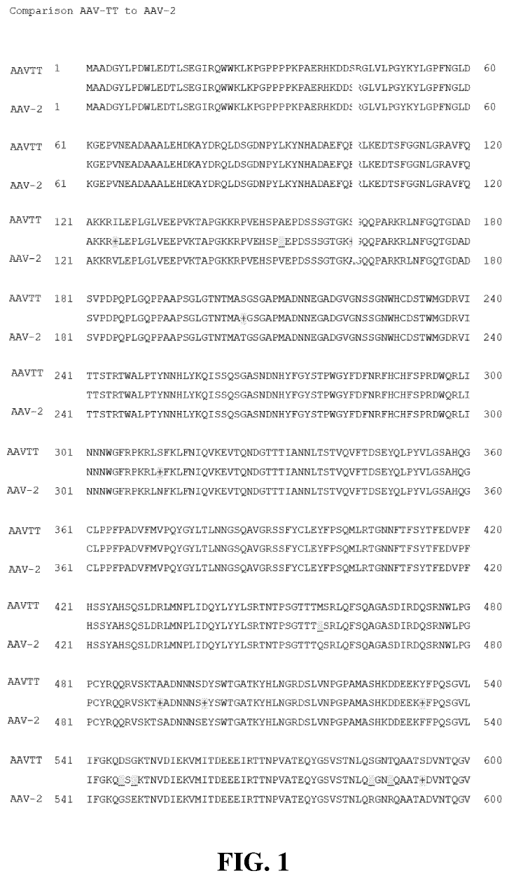

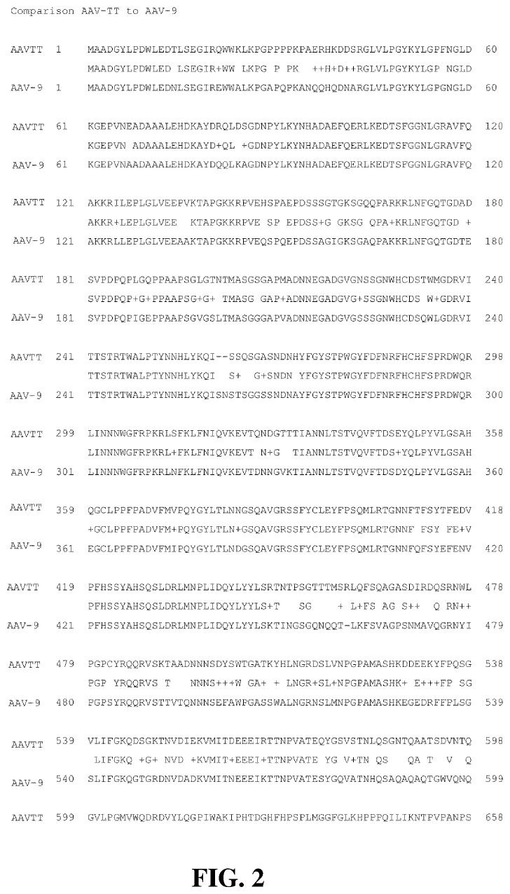

In further embodiments, AAV-TT capsid may be from an AAV serotype other than AAV2 and can be derived for example from AAV1, AAV3B, AAV-LK03, AAV5, AAV6, AAV8, AAV9 or AAV10 capsid protein. In particular, the positions corresponding to those described above with respect to AAV2 can be easily identified by sequence alignments, for example as provided in FIGS. 1 and 2. In one embodiment, AAV-TT VP1 capsid protein of the disclosure comprises or consists of amino acid sequence SEQ ID NO: 14 or an amino acid sequence having at least 95%, 96%, 97%, 98%, preferably 98.5%, more preferably 99% or 99.5% of identity with SEQ ID NO: 14.

In specific embodiments, said AAVretro viral particles are selected according to the present disclosure among those that are able to retrogradely disseminate in the cerebral cortex, preferably at least to the substantia nigra pars compacta and cerebral cortex after intraparenchymal injection in the caudate or putamen nuclei of non-human primate as determined in an in vivo dissemination assay.

In a more specific embodiment, said AAVretro viral particles according to the present disclosure are selected among those which are able to retrogradely disseminate in the cerebral cortex, preferably at least to substantia nigra pars compacta and cerebral cortex after intraparenchymal injection in the caudate or putamen nuclei of non-human primate to at least the same level as AAV-TT as determined in an in vivo dissemination assay.

The inventors indeed designed an in vivo dissemination assay enabling to determine rAAV with true retrograde transport for their use in gene therapies for treating synucleinopathies as disclosed herein, such as Parkinson's disease, and to compare for example with a positive control such as AAV-TT rAAV-GFP.

One important feature of the dissemination assay is that it is an in vivo assay in non-human primate where the rAAV are injected in an area without the presence of fibers of passage. Accordingly, no false positive uptake can be obtained by fibers of passage, i.e. fibers coursing through the injected area towards more distant destination. In non-human primates, the caudate and putamen nuclei are 100% parenchymous structures, and therefore do not contain fibers of passage. Hence, advantageously, suitable rAAV with retrograde transport can be compared and selected according to the present disclosure by means of the proposed dissemination assay.

In a preferred embodiment, said AAV retro viral particle includes AAV TT serotype capsid protein which comprises amino acid sequence SEQ ID NO: 14 or an amino acid sequence having at least 95%, 96%, 97%, 98%, preferably 98.5%, more preferably 99% or 99.5% of identity with SEQ ID NO: 14 and is able to disseminate retrogradely in the cerebral cortex, preferably at least to the substantia nigra pars compacta and cerebral cortex after intraparenchymal injection in the caudate or putamen nuclei of non-human primate as determined in an in vivo dissemination assay.

In a preferred embodiment, said in vivo dissemination assay includes the following steps:
a. injecting a test rAAV comprising a GFP-encoding transgene (rAAV-GFP) by intraparenchymal injection of said rAAV-GFP into the post-commissural putamen of a non-human primate,
b. Counting the number of GFP-expressing neurons in the cerebral cortex, preferably in the brain areas innervating the caudate putamen nuclei one month post injection. GFP encoding transgene may be prepared from GFP encoding nucleic acid of SEQ ID NO: 10 or functional variants thereof with optimized sequence or truncated forms.

Neurons expressing GFP may be visualized by immunoperoxidase stains, using anti-GFP antibodies. GFP-expressing neurons may advantageously be automatically counted throughout the cerebral cortex of the injected non-human primates. A preferential location of GFP-positive neurons is expected to occur in deep layers of the cerebral cortex. Besides cortical areas, GFP-expressing neurons may also be quantified in all brain areas innervating the injected post-commissural putamen or caudate-putamen nuclei, particularly at least the substantia nigra pars compacta, the amygdala and the caudal intralaminar nuclei.

In one specific embodiment, an AAV-retro viral particle according to the present disclosure is selected among those where at least 50%, 60%, 70%, 80% or at least 90% of the neurons of the deep layers V-VI of the cerebral cortex innervating the injected site are expressing GFP as determined in said in vivo dissemination assay. In a preferred embodiment, said AAV retro viral particle includes AAV TT serotype capsid protein which comprises amino acid sequence SEQ ID NO: 14 or an amino acid sequence having at least 95%, 96%, 97%, 98%, preferably 98.5%, more preferably 99% or 99.5% of identity with SEQ ID NO: 14 and where at least 50%, 60%, 70%, 80% or at least 90% of the neurons of the deep layers V-VI of the cerebral cortex innervating the injected site are expressing GFP as determined in said in vivo dissemination assay.

In a more specific embodiment, the dissemination assay is carried out as described in the examples.

In a more specific embodiment, said in vivo dissemination assay includes the following steps:
a. injecting a test rAAV comprising GFP transgene by intraparenchymal injection of said rAAV-GFP into the post-commissural putamen of a non-human primate,
b. counting the number of GFP-expressing neurons in the cerebral cortex, preferably in the brain areas innervating the caudate putamen nuclei, more preferably at least in the cerebral cortex, substantia nigra, amygdala and caudal intralaminar nuclei one month post injection,
c. comparing the percentage of labelled neurons in the cerebral cortex with a control experiment performed with AAV-TT-GFP.

In other embodiments, said AAVretro includes capsid proteins selected among the following variant serotypes: AAV2-retro, AAV-MNM004, AAV-MNM008 and AAV-TT.

In a preferred embodiment, said AAV retro viral particle includes AAV TT serotype capsid protein which comprises amino acid sequence SEQ ID NO: 14 or amino acid sequence having at least 95%, 96%, 97%, 98%, preferably 98.5%, more preferably 99% or 99.5% of identity with SEQ ID NO: 14.

Up to 5 stages of Parkinson's disease have been classically defined by neurologists (see https://www.parkinson.org/Understanding-Parkinsons/What-is-Parkinsons/Stages-of-Parkinsons). The most widely used clinical-rating scale is the so-called Hoehn and Yahr (H-Y) stages that rates the progression of PD in 5 stages: 1 and 2 represent early stages, 2 and 3 mild stages and 4 and 5 advanced stages.

Advantageously, viral particles with retrograde transport enable the viral particles expressing glucocerebrosidase throughout brain areas with disseminated alpha-synuclein aggregates in patients of advanced disease stages.

Accordingly, AAVretro viral particles, preferably AAV retro viral particle which includes AAV TT serotype capsid protein, more preferably comprising amino acid sequence SEQ ID NO: 14 or amino acid sequence having at least 95%, 96%, 97%, 98%, preferably 98.5%, more preferably 99% or 99.5% of identity with SEQ ID NO: 14 will be preferably selected for use according to the present disclosure in treating patients with advanced stages of synucleinopathy, typically, at least H-Y stage 3 of Parkinson disease.

A Process for Producing Viral Particles

Production of viral particles carrying the expression viral vector as disclosed above can be performed by means of conventional methods and protocols, which are selected taking into account the structural features chosen for the actual embodiment of the viral particles to be produced.

Briefly, viral particles can be produced in a host cell, more particularly in specific virus-producing cell (packaging cell), which is transfected with the nucleic acid construct or viral vector to be packaged, in the presence of a helper vector or virus or other DNA construct(s).

The term “packaging cells” as used herein, refers to a cell or cell line which may be transfected with a nucleic acid construct or viral vector of the disclosure, and provides in trans all the missing functions which are required for the complete replication and packaging of a viral vector. Typically, the packaging cells express in a constitutive or inducible manner one or more of said missing viral functions. Said packaging cells can be adherent or suspension cells.

Typically, a process of producing viral particles comprises the following steps:

a) culturing a packaging cell comprising a nucleic acid construct or viral vector as described above in a culture medium; and

b) harvesting the viral particles from the cell culture supernatant and/or inside the cells.

Conventional methods can be used to produce viral particles of the AAV viral particles, which consist on transient cell co-transfection with nucleic acid construct or expression vector (e.g. a plasmid) carrying the transgene encoding glucocerebrosidase; a nucleic acid construct (e.g., an AAV helper plasmid) that encodes rep and cap genes, but does not carry ITR sequences; and with a third nucleic acid construct (e.g., a plasmid) providing the adenoviral functions necessary for AAV replication. Viral genes necessary for AAV replication are referred herein as viral helper genes. Typically, said genes necessary for AAV replication are adenoviral helper genes, such as E1A, E1B, E2a, E4, or VA RNAs. Preferably, the adenoviral helper genes are of the Ad5 or Ad2 serotype.

Large-scale production of AAV particles according to the disclosure can also be carried out for example by infection of insect cells with a combination of recombinant baculoviruses (Urabe et al. Hum. Gene Ther. 2002; 13: 1935-1943). SF9 cells are co-infected with two or three baculovirus vectors respectively expressing AAV rep, AAV cap and the AAV vector to be packaged. The recombinant baculovirus vectors will provide the viral helper gene functions required for virus replication and/or packaging. Smith et al 2009 (Molecular Therapy, vol. 17, no. 11, pp 1888-1896) further describes a dual baculovirus expression system for large-scale production of AAV particles in insect cells.

Suitable culture media will be known to a person skilled in the art. The ingredients that compose such media may vary depending on the type of cell to be cultured. In addition to nutrient composition, osmolarity and pH are considered important parameters of culture media. The cell growth medium comprises a number of ingredients well known by the person skilled in the art, including amino acids, vitamins, organic and inorganic salts, sources of carbohydrate, lipids, trace elements (CuS04, FeS04, Fe(N03)3, ZnS04 . . . ), each ingredient being present in an amount which supports the cultivation of a cell in vitro (i.e., survival and growth of cells). Ingredients may also include different auxiliary substances, such as buffer substances (like sodium bicarbonate, Hepes, Tris or similarly performing buffers), oxidation stabilizers, stabilizers to counteract mechanical stress, protease inhibitors, animal growth factors, plant hydrolyzates, anti-clumping agents, anti-foaming agents. Characteristics and compositions of the cell growth media vary depending on the particular cellular requirements. Examples of commercially available cell growth media are: MEM (Minimum Essential Medium), BME (Basal Medium Eagle) DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12, etc.

Further guidance for the construction and production of viral vectors for use according to the disclosure can be found in Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schafer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 2008; 10:717-733; Adenovirus: Methods and Protocols. M. Chinón and A. Bosch (Eds.); Third Edition. 2014 Humana Press (Springer)

The disclosure also relates to a host cell comprising a nucleic acid construct or a viral vector encoding glucocerebrosidase as described above. More particularly, host cell according to the disclosure is a specific virus-producing cell, also named packaging cell which is transfected with the a nucleic acid construct or a viral vector as described above, in the presence of a helper vector or virus or other DNA constructs and provides in trans all the missing functions which are required for the complete replication and packaging of a viral particle. Said packaging cells can be adherent or suspension cells.

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any packaging cells permissive for baculovirus infection and replication. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, optionally in combination with one or more features of the various embodiments described above or below, the host cell comprises:
a nucleic acid construct or viral vector comprising a transgene encoding glucocerebrosidase as described above (e.g., the AAV vector),
a nucleic acid construct, for example a plasmid, encoding AAV rep and/or cap genes which does not carry the ITR sequences; and, optionally,
a nucleic acid construct, for example a plasmid or virus, comprising viral helper genes.

In another aspect, the disclosure relates to a host cell transduced with a viral particle of the disclosure and the term “host cell” as used herein refers to any cell line that is susceptible to infection by a virus of interest, and amenable to culture in vitro.

Pharmaceutical Compositions

Another aspect of the present disclosure relates to a pharmaceutical composition comprising a nucleic acid construct, a viral vector, a viral particle or a host cell of the disclosure in combination with one or more pharmaceutical acceptable excipient, diluent or carrier.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Preferably, said pharmaceutical composition is formulated as a solution, more preferably as an optionally buffered saline solution. Supplementary active compounds can also be incorporated into the pharmaceutical compositions of the invention. Guidance on co-administration of additional therapeutics can for example be found in the Compendium of Pharmaceutical and Specialties (CPS) of the Canadian Pharmacists Association.

In one embodiment, the pharmaceutical composition is a composition suitable for intraparenchymal, intracerebral, intravenous, or intrathecal administration. These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

Therapeutic Uses

The present invention also provides for the viral particle described herein for use in therapy. In one embodiment, a viral particle for use in therapy is a viral particle that comprises a nucleic acid construct comprising:
a) a transgene encoding a human glucocerebrosidase; wherein said transgene comprises SEQ ID NO: 1, 7, 11, 12 or 19 (preferably SEQ ID NO: 19) or a sequence encoding human glucocerebrosidase, wherein human glucocerebrosidase comprises SEQ ID NO: 5, 6, 8, 17 or 18 (preferably SEQ ID NO: 5 or 8);
b) a promoter operably-linked to said transgene and wherein said promoter preferably allows the expression of said transgene at least in neuronal and microglial cells of the substantia nigra pars compacta (SNc); wherein said promoter is preferably CAG promoter comprising or consisting of SEQ ID NO: 9 or 21, or GusB promoter comprising or consisting of SEQ ID NO: 2 or 20; or JeT promoter comprising or consisting of SEQ ID NO: 27 or hSyn promoter comprising or consisting of SEQ ID NO: 13;
c) a polyadenylation signal sequence, preferably a polyadenylation signal sequence comprising or consisting of SEQ ID NO: 28 or SEQ ID NO: 3, preferably SEQ ID NO: 28;

wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16.

Using animal models of sporadic Parkinson's disease in mice and non-human primates, the inventors surprisingly found that AAV-mediated enhancement of glucocerebrosidase activity:
induces alpha-synuclein aggregates clearance in dopaminergic neurons of the substantia nigra pars compacta;
induces neuroprotection of dopaminergic neurons,
attenuates microglia-driven pro-inflammatory phenomena triggered by alpha-synuclein aggregation, and
impedes the trans-neuronal passage of alpha-synuclein (prion-like spread).

These results provide strong evidence of a possible therapeutic strategy for treating synucleinopathies, in particular sporadic synucleinopathies, and more specifically Parkinson's disease in human subject.

Therefore, in a further aspect, there is provided a viral particle or viral vector as described herein for use in the treatment of synucleinopathy, preferably Parkinson's disease, and more specifically sporadic Parkinson's disease.

In one embodiment, the viral particle for use in the treatment of synucleinopathy, preferably Parkinson's disease, and more specifically sporadic Parkinson's disease, is a viral particle comprising a nucleic acid construct comprising:
a) a transgene encoding a human glucocerebrosidase; wherein said transgene comprises SEQ ID NO: 1, 7, 11, 12 or 19 (preferably SEQ ID NO: 19) or a sequence encoding human glucocerebrosidase, wherein human glucocerebrosidase comprises SEQ ID NO: 5, 6, 8, 17 or 18 (preferably SEQ ID NO: 5 or 8);
b) a promoter operably-linked to said transgene and wherein said promoter preferably allows the expression of said transgene at least in neuronal and microglial cells of the substantia nigra pars compacta (SNc); wherein said promoter is preferably CAG promoter comprising or consisting of SEQ ID NO: 9 or 21, or GusB promoter comprising or consisting of SEQ ID NO: 2 or 20; or JeT promoter comprising or consisting of SEQ ID NO: 27 or hSyn promoter comprising or consisting of SEQ ID NO: 13;
c) a polyadenylation signal sequence, preferably a polyadenylation signal sequence comprising or consisting of SEQ ID NO: 28 or SEQ ID NO: 3, preferably SEQ ID NO: 28;

wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16.

In addition, the disclosure relates to a method for treating synucleinopathy, preferably Parkinson's disease, and more specifically sporadic Parkinson's disease, in a subject in need thereof, said method comprising administering to said subject a therapeutically effective amount of a viral particle or viral vector as described above.

In one embodiment, the method for treating synucleinopathy, preferably Parkinson's disease, and more specifically sporadic Parkinson's disease, in a subject in need thereof, said method comprises administering to said subject a therapeutically effective amount of a viral particle comprising a nucleic acid construct comprising:
a) a transgene encoding a human glucocerebrosidase; wherein said transgene comprises SEQ ID NO: 1, 7, 11, 12 or 19 (preferably SEQ ID NO: 19) or a sequence encoding human glucocerebrosidase, wherein human glucocerebrosidase comprises SEQ ID NO: 5, 6, 8, 17 or 18 (preferably SEQ ID NO: 5 or 8);
b) a promoter operably-linked to said transgene and wherein said promoter preferably allows the expression of said transgene at least in neuronal and microglial cells of the substantia nigra pars compacta (SNc); wherein said promoter is preferably CAG promoter comprising or consisting of SEQ ID NO: 9 or 21, or GusB promoter comprising or consisting of SEQ ID NO: 2 or 20; or JeT promoter comprising or consisting of SEQ ID NO: 27 or hSyn promoter comprising or consisting of SEQ ID NO: 13;
c) a polyadenylation signal sequence, preferably a polyadenylation signal sequence comprising or consisting of SEQ ID NO: 28 or SEQ ID NO: 3, preferably SEQ ID NO: 28;
   wherein said viral particle is a recombinant Adeno-Associated Virus (rAAV) particle, preferably including capsid proteins of AAV TT, and more preferably comprising SEQ ID NO:

14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16.

In a particular embodiment, said method comprises administering to a subject a therapeutically effective amount of a viral particle or viral vector as described above to be delivered to neurons of cerebral cortex, preferably neurons of the deep layers V-VI of the cerebral cortex, preferably at least 10%, 20%, 30%, 40%, 50%, 60%, 70%, 80% or at least 90% of the neurons of the deep layers V-VI of the cerebral cortex innervating the administrated site.

In another particular embodiment, said method comprises administering to a subject a therapeutically effective amount of a viral particle or viral vector as described above to be delivered to neurons of brain areas innervating the injection site, preferably to be delivered to neurons of at least brain areas innervating the caudate-putamen nuclei, i.e. at least substantia nigra pars compacta, cerebral cortex, amygdala and caudal intralaminar nuclei of the thalamus, preferably at least 10%, 20%, 30%, 40%, 50%, 60%, 70%, 80% or at least 90% of these neurons.

In a further aspect, the disclosure relates to a nucleic acid construct, viral vector, viral particle, host cell or pharmaceutical composition as described above, for use as a medicament in a subject in need thereof, and more specifically, for use in treating synucleinopathy, preferably Parkinson's disease, and more specifically sporadic Parkinson's Disease in a subject in need thereof.

In another further aspect, the disclosure relates to the use of a nucleic acid construct, viral vector, viral particle, host cell or pharmaceutical composition as described above in the manufacture of a medicament, preferably for treating synucleinopathy, preferably Parkinson's disease, and more specifically sporadic Parkinson's disease.

The term “subject” or “patient” as used herein, refers to mammals. Mammalian species that can benefit from the disclosed methods of treatment include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. In particular embodiment, said subject is neonate, an infant or, a child.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

As used herein, synucleinopathies refer to diseases where the neuropathological hallmark is represented by the intracytoplasmic aggregation of alpha-synuclein. In particular, synucleinopathies include neurodegenerative disorders such as Parkinson's disease, dementia with Lewy bodies, and multiple system atrophy.

As used herein, Parkinson's disease (PD) refers to a progressive neurodegenerative disorder of the central nervous system of an unknown origin. Within the context of PD, dopamine-producing neurons progressively die, leading to a brain deficiency in a neurotransmitter known as dopamine. As a result of the progressive decline in dopamine brain levels, brain circuits controlling initiation and execution of voluntary movements become dysfunctional, therefore leading to the appearance of the cardinal motor symptoms that typically characterize PD. Initial diagnosis usually takes place in the sixth decade of life spam (65 years of age, on average), and the characteristic presentation is unilateral and distal. As the disease progresses, the disease affects both sides of the body (e.g. bilateral) and symptoms became worse and more apparent over time. Upon initial diagnosis, most patients are facing a variable period of time (approximately between 5 and 7 years) where the disease can be managed pharmacologically by taking medication such as different formulations of levodopa (a dopamine precursor) and/or a wide variety of dopaminergic agonists. As the disease goes on, medication dosage increases to counteract the unrelenting loss of dopaminergic neurons to a time point where PD is no longer manageable with dopamine-replacement therapies because the appearance of on-off phenomena and side effects related to the chronic intake of medication (levodopa-induced dyskinesia). At this stage, patients can be treated with functional neurosurgical approaches, consisting in the bilateral placement of electrodes within the basal ganglia circuits (a procedure known as deep brain stimulation). Regardless the therapeutic options, available approaches are merely symptomatic, i.e. can alleviate motor-related symptoms without any effect on disease progression rates. The typical symptomatic triad is made of tremor (shaking), bradykinesia (slowness of movements), and rigidity (because of muscle stiffness). Besides the typical triad, few more symptoms are often noticed, these including gait disturbances, dysarthria (speech disturbances), pain, as well as a large plethora of non-motor symptoms (constipation, urinary incontinence, REM sleep disturbances, olfactory dysfunction, and orthostatic hypotension, among others). Psychiatric symptoms also often appear in parallel to disease progression and may include disorders of cognition, thought, mood and behavior. Indeed, dementia is often seen in later stages of the disease.

The main neuropathological hallmark of PD is represented by the intracytoplasmic aggregation of a misfolded protein known as alpha-synuclein. Alpha-synuclein aggregates are often seen as spheroid-like structures called Lewy bodies as well as aberrant neuronal structures (Lewy body neurites). The presence of Lewy bodies within dopaminergic neurons in a brain area known as the substantia nigra pars compacta is the criteria of choice for the neuropathological confirmation of PD. Diagnosis of PD is made after clinical evaluation carried out by neurologists, by balancing together the clinical phenotype. In parallel, neuroimage studies with positron emission tomography (PET scans) using the radiotracer fluoro-dopa (a dopamine analogue) also supports the initial diagnosis of PD and are indeed very useful as a neuroimage correlate of disease progression over time.

The above method is particularly suitable for treating sporadic synucleinopathies, in particular sporadic Parkinson's disease. As used herein, sporadic synucleinopathy (also referred as idiopathic disorders) refers to synucleinopathy which is not associated to known particular genetic mutations (familial case). Such known genetic mutations associated to familial synucleinopathies include a mutation in a gene selected from the group consisting of LRRK2, SNCA, VPS35, GCH1, ATXN2, DNAJC13, TMEM230, GIGYF2, HTRA2, RIC3, EIF4G1, UCHL1, CHCHD2, GBA1, PRKN, PINK1, DJ1, ATP13A2, PLA2G6, FBXO7, DNAJC6, SYNJ1, SPG11, VPS13C, PODXL, PTRHD1, RAB39B, DNAJC13, TMEM230, GIGYF2, HTRA2, RIC3, EIF4G1, UCHL1, and CHCHD2. These mutations are further described in more detail in Lunati et al, The genetic landscape of Parkinson's disease, Rev Neurol 2018; 174:628-643.

The synucleinopathies as described above may be associated with defects in lysosomal storage, particularly Gaucher disease. Thus, in another particular embodiment, the above method is also suitable for treating neuronopathic Gaucher disease, more particularly Gaucher disease of type II or type III in a subject in need thereof, said method comprising administering to said subject a therapeutically effective amount of a viral particle, viral vector, host cell or pharmaceutical composition as described above.

Gaucher disease (GD) refers to a lysosomal storage disease, more specifically a sphingolipidoses that is characterized by the deposition of glucocerebroside in cells of the macrophage-monocyte system.

The term “lysosomal storage disease” as used herein refers to genetic diseases and metabolic disorders that result from defects in lysosomal function. Lysosomal storage disorders are caused by lysosomal dysfunction usually as a consequence of deficiency of a single enzyme required for the metabolism of lipids, glycoproteins or so-called mucopolysaccharides that induce an abnormal accumulation of substances inside the lysosome.

Gaucher disease is caused by a recessive mutation(s) in the gene coding for the enzyme glucocerebrosidase. Different mutations in the beta-glucosidase determine the remaining activity of the enzyme, and, to a large extent, the phenotype. Gaucher's disease has three common clinical subtypes, non-neuronopathic type I, which is the most common form of the disease, acute neuronopathic type II, also referred to herein as type II and chronic neuronopathic type III, also referred to herein as type III. Gaucher disease of type II (acute neuronopathic) or III (subacute neuronopathic) is characterized by the presence of primary neurologic disease affecting the central nervous system. Gaucher disease of type II can begin at any time in childhood, as early as 6 months after birth and occurs in approximately 1 in 100,000 live births.

It is characterized severe neurological involvement of the brainstem, associated with an organomegaly and generally leading to death before the age of 2. Major symptoms include an enlarged spleen and/or liver, seizures, poor coordination, skeletal irregularities, eye movement disorders, blood disorders including anemia and respiratory problems. Gaucher disease of type III, occurs later in childhood and adolescence and has an incidence rate of approximately 1 in 100,000 live births. Symptoms progress more slowly in comparison to type II and include an enlarged liver and spleen, extensive and progressive brain damage, eye movement disorders, spasticity, seizures, limb rigidity, and a poor ability to suck and swallow.

In a further aspect, the disclosure relates to viral particle, viral vector, host cell or pharmaceutical composition as described above, for use as a medicament in a subject in need thereof, and more specifically, for use in treating neuronopathic Gaucher disease, more particularly Gaucher disease of type II or type III in a subject in need thereof.

In another further aspect, the disclosure relates to the use of a nucleic acid construct, viral vector, viral particle, host cell or pharmaceutical composition as described above in the manufacture of a medicament, preferably for treating neuronopathic Gaucher disease, more particularly Gaucher disease of type II or type III.

In one embodiment, the viral particle for use in the treatment of neuronopathic Gaucher disease, more particularly Gaucher disease of type II or type III, is a viral particle comprising a nucleic acid construct comprising:
d) a transgene encoding a human glucocerebrosidase; wherein said transgene comprises SEQ ID NO: 1, 7, 11, 12 or 19 (preferably SEQ ID NO: 19) or a sequence encoding human glucocerebrosidase, wherein human glucocerebrosidase comprises SEQ ID NO: 5, 6, 8, 17 or 18 (preferably SEQ ID NO: 5 or 8);
e) a promoter operably-linked to said transgene and wherein said promoter preferably allows the expression of said transgene at least in neuronal and microglial cells of the substantia nigra pars compacta (SNc); wherein said promoter is preferably CAG promoter comprising or consisting of SEQ ID NO: 9 or 21, or GusB promoter comprising or consisting of SEQ ID NO: 2 or 20; or JeT promoter comprising or consisting of SEQ ID NO: 27 or hSyn promoter comprising or consisting of SEQ ID NO: 13;
f) a polyadenylation signal sequence, preferably a polyadenylation signal sequence comprising or consisting of SEQ ID NO: 28 or SEQ ID NO: 3, preferably SEQ ID NO: 28;
wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16.

In addition, the disclosure relates to a method for treating neuronopathic Gaucher disease, more particularly Gaucher disease of type II or type III, in a subject in need thereof, said method comprising administering to said subject a therapeutically effective amount of a viral particle or viral vector as described above.

In one embodiment, the method for treating neuronopathic Gaucher disease, more particularly Gaucher disease of type II or type III, in a subject in need thereof, said method comprises administering to said subject a therapeutically effective amount of a viral particle comprising a nucleic acid construct comprising:
a) a transgene encoding a human glucocerebrosidase; wherein said transgene comprises SEQ ID NO: 1, 7, 11, 12 or 19 (preferably SEQ ID NO: 19) or a sequence encoding human glucocerebrosidase, wherein human glucocerebrosidase comprises SEQ ID NO: 5, 6, 8, 17 or 18 (preferably SEQ ID NO: 5 or 8);
b) a promoter operably-linked to said transgene and wherein said promoter preferably allows the expression of said transgene at least in neuronal and microglial cells of the substantia nigra pars compacta (SNc); wherein said promoter is preferably CAG promoter comprising or consisting of SEQ ID NO: 9 or 21, or GusB promoter comprising or consisting of SEQ ID NO: 2 or 20; or JeT promoter comprising or consisting of SEQ ID NO: 27 or hSyn promoter comprising or consisting of SEQ ID NO: 13;
c) a polyadenylation signal sequence, preferably a polyadenylation signal sequence comprising or consisting of SEQ ID NO: 28 or SEQ ID NO: 3, preferably SEQ ID NO: 28;

wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16.

As used herein a “therapeutically effective amount” refers to an amount effective, at dosages and for periods of time necessary to achieve the desired therapeutic result, such as one or more of the following therapeutic results:
a significant reduction of alpha-synuclein burden in dopaminergic neurons of the substantia nigra compacta in said subject, and preferably also in neurons of any other brain area showing alpha-synuclein aggregates,
a significant neuroprotective effect of dopaminergic neurons in the substantia nigra compacta, as shown by a significant reduction of death in tyrosine-positive neurons,
a significant neuroprotective effect in neurons of any other brain area showing alpha-synuclein aggregates,
a significant decrease of microglia-driven proinflammatory phenomena triggered by alpha-synuclein aggregation,
a significant blockade of the prion-like trans-neuronal passage of alpha-synuclein.

As used, the “prion-like trans-neuronal passage of alpha-synuclein” refers to the ability of alpha-synuclein for propagating from a neuronal axon terminal into the next neuron being innervated by the alpha-synuclein-expressing axon terminal.

A significant reduction of alpha-synuclein burden (e.g. in dopaminergic neurons of the substantia nigra pars compacta) may correspond to a reduction of at least 10%, 20%, 30%, 40%, 50%, 60%, 70%, 80%, or at least 90% of alpha-synuclein aggregates in the corresponding brain area (e.g. substantia nigra pars compacta) after a minimum period of 4 weeks of treatment.

In some embodiments, a significant neuroprotective effect of dopaminergic neurons in a treated patient may be estimated as at least 10%, at least 20% or at least 30% improved neuronal survival vs untreated patients after a minimal period of 52 weeks (a year) of treatment.

In other specific embodiments, a treatment with a product of the disclosure may inhibit the progression or delay the onset, or reduce the severity of one or more symptoms of synucleinopathies or neuronopathic Gaucher disease. For example, a treatment may inhibit the progression, or delay the onset, or reduce the severity of one or more of the following symptoms:
degeneration of dopaminergic neurons (e.g. in the substantia nigra pars compacta),
bradykinesia
muscle rigidity
tremors, rest tremors,
impaired balance and gait disturbances
neuropsychiatric symptoms,
accumulation of alpha-synuclein)
accumulation of Lewy bodies
Disease progression rates
Scores obtained in clinical motor-related scales
Scores obtained in tests measuring cognitive status
Levels of radiotracer uptake in neuroimage PET scans (measured as binding potential)
Olfactory tests
REM sleep disturbances
Non-motor symptoms (constipation, urinary incontinence, REM sleep disturbances, olfactory dysfunction, and orthostatic hypotension, among others)
Dysarthria and speech fluency
Disorders of cognition (including progression to dementia), thought, mood and behavior.

In one embodiment, an effective amount of the viral particle (or viral vector) as described above is administered to the subject or patient by intraparenchymal, intracerebral, intracerebroventricular (icv), intrathecal, or intravenous route.

In some embodiment, for Parkinson's disease, in particular for sporadic Parkinson's disease the targeted area is represented by any brain area showing accumulation of alpha-synuclein, in particular the substantia nigra pars compacta and cerebral cortex. Hence, a therapeutically effective amount of said viral particle or viral vector is preferably administered by intraparenchymal route, more preferably to the brain area of the substantia nigra pars compacta and/or the caudate putamen nuclei. In one embodiment, the intraparenchymal route may facilitate preferred local administration to the SNc as compared to other area of the brain.

As used herein, a “preferred local administration to the SNc” does not mean that all the viral particles or viral vectors are administered to the SNc, but a majority, for example at least 50%, at least 60%, at least 70%, or at least 80% (vg) of the viral particles are administered either to the area of the SNc or to any other brain area innervated by SNc neurons.

With administration in the cerebrospinal space, neuronal transduction is dependent of cerebrospinal fluid circulation dynamics, therefore is expected to occur (1) in periventricular areas, i.e. areas in close proximity to the cerebral ventricles, (2) through a non-specific manner, i.e. neurons will be transduced by diffusion either from the ventricles or from the subarachnoid space, with strong labeling expected to be observed in upper cortical layers I-IV (e.g. by diffusion from the subarachnoid space) and (3) in brain areas such as the cerebellum and the hippocampus that are not connected to the putamen. Transduction from the ventricular system of neurons of deep brain areas such as the substantia nigra would be very unlikely bearing in mind that the substantia nigra is located far away from the ventricles, therefore very difficult to be transfected by passive diffusion.

In contrast to the administration in the cerebrospinal space, the administration of a viral vector in the caudate putamen nuclei presents several advantages such as a specific transduction of neurons located in cerebral cortex, thalamus, amygdala, substantia nigra pars compacta and dorsal raphe nuclei innervating the injection site and circuit-specific retrograde spread in brain areas known to innervate the putamen for instance in layer V of the cortical areas projecting to the putamen without retrograde spread to unexpected areas (e.g. lack of retrograde transport to areas known to not to innervate the putamen).

Thus, the intraparenchymal route may facilitate local administration of a viral particle to the caudate-putamen nuclei, thus facilitating retrograde dissemination of a transgene to any brain area innervating the injection site.

In a preferred embodiment, a viral particle can be administered to the human subject or patient via intraparenchymal route to the caudate-putamen nuclei, in a volume comprised within a range of 50 to 100 μL, preferably 200 to 70 μL per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours. Such a high injection debit of the viral particle increases virus stability and allows a better management of patients.

In certain embodiments, said viral particle is selected among rAAV particles, preferably including capsid proteins selected from the group consisting of: AAV2, AAV5, AAV9, AAV-MNM004, AAV-MNM008, and AAV TT serotypes.

In certain embodiments, said viral particle is an AAVretro which includes capsid proteins selected among the following variant serotypes: AAV2-retro, AAV-MNM004, AAV-MNM008 and AAV-TT.

In one embodiment, AAV-TT particle can be administered to the human subject or patient via intraparenchymal route to the caudate-putamen nuclei, in a volume comprised within a range of 50 to 100 μL, preferably 200 to 70 μL per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours.

In another embodiment, an AAV-9 particle can be administered to the human subject or patient via intraparenchymal route to the caudate-putamen nuclei, in a volume comprised within a range of 50 to 100 μL, preferably 200 to 70 μL per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours.

In one embodiment the intraparenchymal route may facilitate preferred local administration of an AAV to the caudate-putamen nuclei, thus facilitating retrograde dissemination of GBA1 transgene to any brain area innervating the injection site.

The present disclosure relates to a viral particle, preferably AAV particle comprising GBA1 transgene according to the present disclosure for use in the treatment of a neurodegenerative disease such as synucleinopathies wherein said viral particle is administered via intraparenchymal route to the caudate-putamen nuclei.

In a preferred embodiment, an AAV viral particle according to the disclosure can be administered to the human subject or patient for the treatment of synucleinopathies, such as Parkinson's Disease or neuronopathic Gaucher disease via intraparenchymal route to the caudate-putamen nuclei, in a volume comprised within a range of 50 to 1000 μL, preferably 200 to 700 μL per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours.

In a particular embodiment, an AAV-TT according to the present disclosure can be administered to the human subject or patient for the treatment of synucleinopathies, such as Parkinson's Disease or neuronopathic Gaucher disease via intraparenchymal route to the caudate-putamen nuclei.

Said AAV-TT particle according to the disclosure can be administered to the human subject or patient for the treatment of synucleinopathies, such as Parkinson's Disease or neuronopathic Gaucher Disease via intraparenchymal route to the caudate-putamen nuclei, in a volume comprised within a range of 50 to 1000 μL, preferably 200 to 700 μK per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours.

In a preferred embodiment, there is provided an recombinant Adeno-Associated Virus (rAAV) particle comprising a nucleic acid construct comprising a transgene which comprises a nucleotide sequence selected from the group consisting of SEQ ID NO: 1, 7 11, 12 and 19 or a nucleotide sequence encoding human glucocerebrosidase comprising SEQ ID NO: 5, 6, 8, 17 or 18, wherein said nucleic acid construct further comprises a promoter operably-linked to said transgene, wherein said rAAV particle include AAV-TT capsid proteins comprising amino acid sequence of SEQ ID NO: 14 or sequence having at least 95%, 96%, 97%, 98%, preferably 98.5%, more preferably 99% or 99.5% identity with SEQ ID NO: 14, for use in the treatment of a neurodegenerative disease such as synucleinopathies, preferably Gaucher disease (such as neuropathic Gaucher disease) or PD (such as sporadic PD), wherein said rAAV particle is administered via intraparenchymal route to the caudate-putamen nuclei, preferably in a volume comprised within a range of 50 to 100 μL, preferably 200 to 70 μL per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours.

In a further preferred embodiment, the present disclosure relates to a viral particle comprising a nucleic acid construct comprising:
a) a transgene encoding a human glucocerebrosidase; wherein said transgene comprises SEQ ID NO: 1, 7, 11, 12 or 19 (preferably SEQ ID NO: 19) or a sequence encoding human glucocerebrosidase, wherein human glucocerebrosidase comprises SEQ ID NO: 5, 6, 8, 17 or 18 (preferably SEQ ID NO: 5 or 8);
b) a promoter operably-linked to said transgene and wherein said promoter preferably allows the expression of said transgene at least in neuronal and microglial cells of the substantia nigra pars compacta (SNc); wherein said promoter is preferably CAG promoter comprising SEQ ID NO: 9 or 21, or GusB promoter comprising SEQ ID NO: 2 or 20 or JeT promoter comprising or consisting of SEQ ID NO: 27 or hSyn promoter comprising SEQ ID NO: 13;
c) a polyadenylation signal sequence, preferably a polyadenylation signal sequence comprising or consisting of SEQ ID NO: 28 or SEQ ID NO: 3, preferably SEQ ID NO: 28;

wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16; wherein said viral particle is for use in the treatment of a neurodegenerative disease such as synucleinopathies, preferably Gaucher disease (such as neuropathic Gaucher disease) or PD (such as sporadic PD), wherein said rAAV particle is administered via intraparenchymal route to the caudate-putamen nuclei, preferably in a volume comprised within a range of 50 to 100 μL, preferably 200 to 700 μL per putamen, at a concentration preferably comprised within the range of 10¹³-10¹⁴ vg/mL (vg: viral genomes).

In a particular embodiment, said viral particle is administered at an injection debit comprised within the range of 0.5 to 5 μL/min preferably during 2 to 6 hours.

Furthermore, in a further preferred embodiment, there is provided a method for treating synucleinopathies, preferably Gaucher disease (such as neuropathic Gaucher disease) or PD (such as sporadic PD) to a subject in need thereof, said method comprises administering to said subject a therapeutically effective amount of a viral particle comprising a nucleic acid construct comprising:
a) a transgene encoding a human glucocerebrosidase; wherein said transgene comprises SEQ ID NO: 1, 7, 11, 12 or 19 (preferably SEQ ID NO: 19) or a sequence encoding human glucocerebrosidase, wherein human glucocerebrosidase comprises SEQ ID NO: 5, 6, 8, 17 or 18 (preferably SEQ ID NO: 5 or 8);
b) a promoter operably-linked to said transgene and wherein said promoter preferably allows the expression of said transgene at least in neuronal and microglial cells of the substantia nigra pars compacta (SNc); wherein said promoter is preferably CAG promoter comprising SEQ ID NO: 9 or 21, or GusB promoter comprising SEQ ID NO: 2 or 20 or JeT promoter comprising or consisting of SEQ ID NO: 27 or hSyn promoter comprising SEQ ID NO: 13;
c) a polyadenylation signal sequence, preferably a polyadenylation signal sequence comprising or consisting of SEQ ID NO: 28 or SEQ ID NO: 3, preferably SEQ ID NO: 28;

wherein said viral particle is a recombinant Adeno-Associated Virus (rAAV) particle, preferably including capsid proteins of AAV TT, and more preferably comprising SEQ ID NO: 14 or a sequence having at least 95%, 96%, 97%, 98%, preferably 98.5%, more preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16; wherein said rAAV particle is administered via intraparenchymal route to the caudate-putamen nuclei, preferably in a volume comprised within a range of 50 to 100 μL, preferably 200 to 700 μL per putamen, at a concentration preferably comprised within the range of 10¹³-10¹⁴ vg/mL (vg: viral genomes).

In a particular embodiment, said viral particle is administered at an injection debit comprised within the range of 0.5 to 5 μL/min preferably during 2 to 6 hours. In another embodiment, an AAV-9 according to the present disclosure can be administered to the human subject or patient for the treatment of synucleinopathies, such as Parkinson's Disease or neuronopathic Gaucher disease, via intraparenchymal route to the caudate-putamen nuclei.

Said AAV-9 particle according to the disclosure can be administered to the human subject or patient for the treatment of synucleinopathies, such as Parkinson's Disease or neuronopathic Gaucher disease via intraparenchymal route to the caudate-putamen nuclei, in a volume comprised within a range of 50 to 100 μL, preferably 200 to 700 μL per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours.

The therapeutically effective amount of the product of the disclosure, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response.

A therapeutically effective amount is also typically one in which any toxic or detrimental effect of the product or pharmaceutical composition is outweighed by the therapeutically beneficial effects.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

In one embodiment, an AAV viral particle according to the disclosure can be administered to the human subject or patient for the treatment of synucleinopathies, such as sporadic Parkinson's Disease or neuronopathic Gaucher disease, in an amount or dose comprised within a range of 10⁸-10¹⁴ vg/kg (vg: viral genomes; kg: subject's or patient's body weight). In a more particular embodiment, the AAV viral particle is administered in an amount comprised within a range of 10⁹-10¹³ vg/kg. In a more particular embodiment, the AAV viral particle is administered at a dosage of at least 10¹⁰-10¹² vg/kg in a human subject.

Furthermore, multiple doses of such viral particles may be administered to a human subject simultaneously or sequentially, in particular to ensure a homogeneous distribution of the vector through the area of delivery, e.g. the substantia nigra pars compacta and/or the caudate-putamen nuclei. Typically, 3, 4, 5 or more injections of a dose of viral particles may be administered at the same time point to the area of delivery, e.g. the substantia nigra pars compacta and/or the caudate-putamen nuclei of a human subject, for example using intraparenchymal route.

Kit

In another aspect, the disclosure further relates to a kit comprising a nucleic acid construct, viral vector, a host cell, viral particle or pharmaceutical composition as described above in one or more containers. The kit may include instructions or packaging materials that describe how to administer the nucleic acid construct, viral vector, viral particle, host cell or pharmaceutical composition contained within the kit to a patient. Containers of the kit can be of any suitable material, e.g., glass, plastic, metal, etc., and of any suitable size, shape, or configuration. In certain embodiments, the kits may include one or more ampoules or syringes that contain the products of the invention in a suitable liquid or solution form.

The following examples are provided by way of illustration, and they are not intended to be limiting of the present disclosure.

### EXAMPLES

In Vivo Dissemination Assay for Testing and Comparing AAV with Retrograde Transport

A test rAAV-GFP was prepared using standard methods for producing rAAV. The test rAAV-GFP used a nucleic acid construct with a GFP encoding sequence as the transgene, under the control of CAG promoter, and with ITRs of AAV2, said nucleic acid construct being packaged with capsid proteins of the AAV serotype to be tested for its retrograde transport property.

The in vivo dissemination assay included a first step of injecting said test rAAV-GFP by intraparenchymal injection of said rAAV-GFP into the post-commissural putamen of a non-human primate.

Then, the assay included a step of counting the number of GFP-expressing neurons in the cerebral cortex, substantia nigra, amygdala and caudal intralaminar nuclei one month post injection. Cell counting were carried out by taking advantage of Aiforia™, a whole-slide digital imaging and deep convolutional neuronal networks (CNN) algorithm designed for the automatic unbiased counting of immunoperoxidase-stained cells in brain tissue specimens (Penttinen et al., European Journal of Neuroscience 2018; 48:2354-2361).

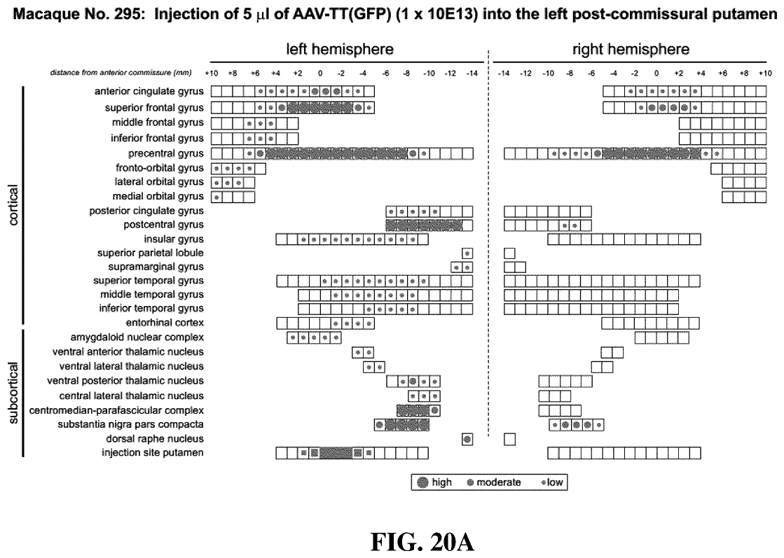

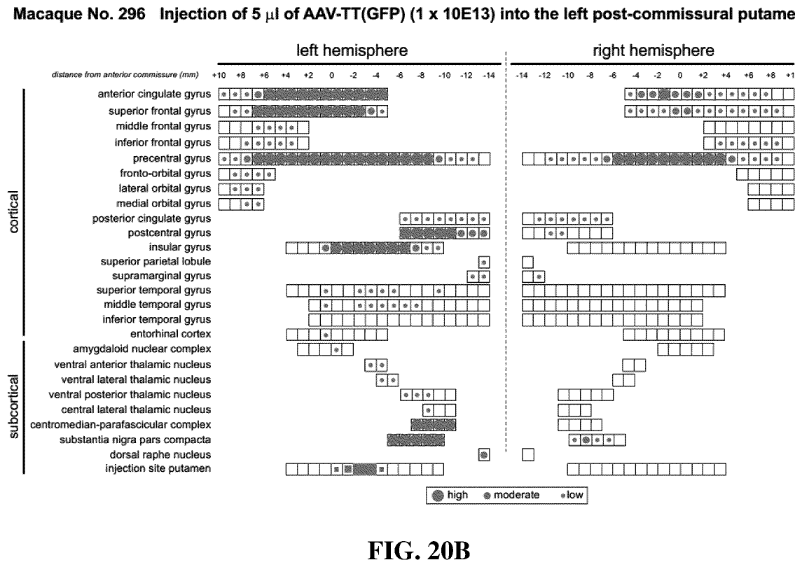

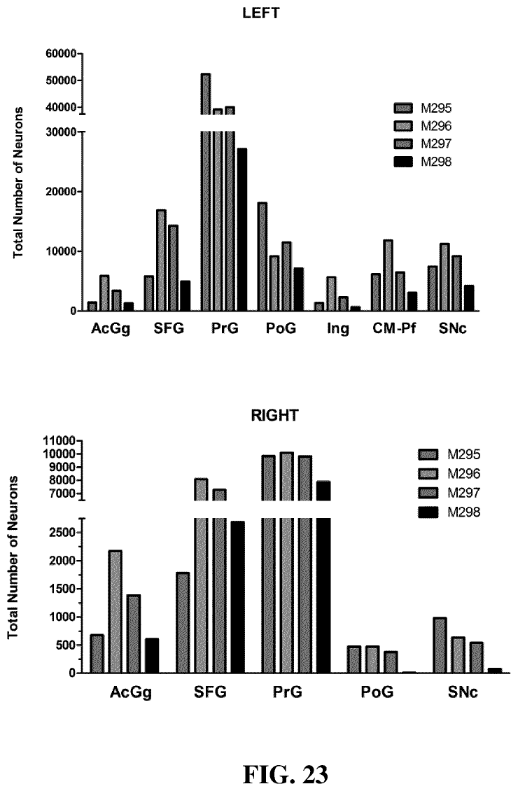

Neurons expressing GFP were visualized by immunoperoxidase stains, using anti-GFP antibodies. GFP-expressing neurons were automatically counted throughout the brain of the injected non-human primates. A preferential location of GFP-positive neurons occurred in deep layers of the cerebral cortex (e.g. layers V and VI) as illustrated in FIGS. 20 and 23. Besides cortical areas, GFP-expressing neurons were quantified in all brain areas innervating the injected post-commissural putamen, particularly the substantia nigra pars compacta, the amygdala and the caudal intralaminar nuclei (FIGS. 20 and 23).

1. Studies in Mice:

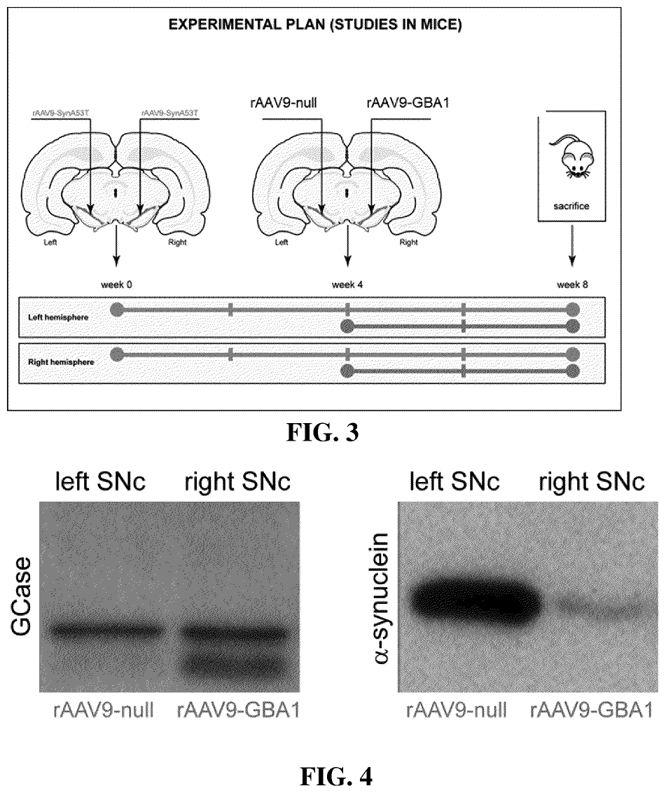

Wild-type mice (n=11) were injected bilaterally into the SNc through stereotaxic surgery with a recombinant AAV serotype 9 coding for the mutated form of human alpha-synuclein under the control of a synapsin neuron specific promoter (rAAV2/9-SynA53T). Each SNc received 1.0 microliters of 1.36×10E13 of the viral suspension. Once the neurodegenerative processes are already ongoing but before reaching a non-returning point (e.g. 4 weeks post-delivery of rAAV2/9-SynA53T), a recombinant AAV9 coding for the GBA1 gene under the control of a constitutive promoter GusB (interchangeably named in the examples and figures as rAAV9-GBA1 or rAAV2/9-GBA1) was delivered into the right SNc (1.0 microliters of 1.25×10E13 of the viral suspension), together with an empty, control rAAV9 coding for no transgene (interchangeably named in the examples and figures as rAAV9-null or r-AAV2/9-null) injected into the left SNc. 4 weeks later (e.g. 8 weeks after initial delivery of rAAV2/9-SynA53T), animals were sacrificed and processed for neuropathological analysis. The conducted experimental plan is summarized below (FIG. 3).

Western Blot analysis (see FIG. 4) conducted in an additional cohort of 5 mice showed (i) enhanced GCase protein levels expression in the right SNc (e.g. the one injected with rAAV2/9-GBA1) and (ii) a clear reduction in alpha-synuclein protein levels in the right SNc.

Neuropathological Data:

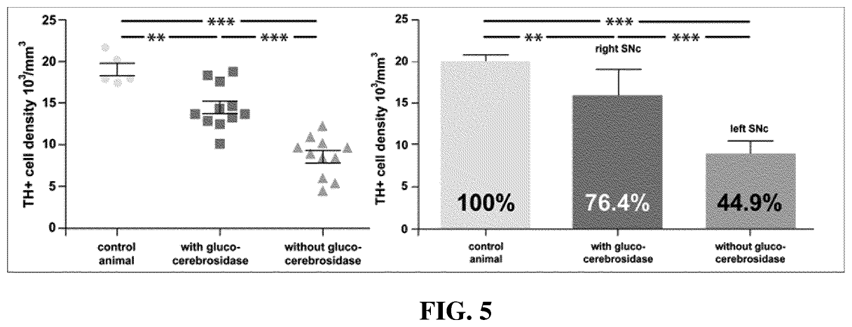

The in-depth neuropathological analysis conducted revealed that (1) the rAAV2/9-GBA1 mediated enhancement of GCase resulted in an almost complete clearance of alpha-synuclein (Data not shown). (2) alpha-synuclein clearance also comprises phosphorylated (e.g. aggregated) forms of alpha-synuclein, together with an evident neuroprotective effect exerted on dopaminergic neurons in keeping with increased expression levels of GCase (Data not shown). (3) the unbiased stereological estimation of the density of tyrosine-positive neurons in the SNc showed an statistically very significant difference when comparing left vs. right SNc. Upon a follow-up of 8 weeks post-delivery of rAAV2/9-SynA53T into the left SNc, roughly a 55% of neuronal death was observed, contrasting with a 24% of neuronal death being noticed in the right SNc, i.e. the one treated with rAAV2/9-GBA1 (FIG. 5). (4) Microglial-driven pro-inflammatory phenomena were attenuated in the right SNc upon the delivery of rAAV2/9-GBA1. As soon as dopaminergic neurons started expressing alpha-synuclein, microglial cells change phenotype and enveloped alpha-synuclein expressing neurons, likely in an attempt to isolate these neurons from the surrounding. This switch in the morphological phenotype of microglial cells is reverted back to normal morphology upon AAV-mediated GCase enhancement (Data not shown). Analysis of microglial phenotypes by comparing treated vs. untreated sides of the SNc has been carried out using antibodies against the ionized calcium-binding adaptor molecule 1 (Iba1), a microglial and macrophage-specific calcium-binding protein that is involved with the membrane ruffling and phagocytosis in activated microglia.

Taken together obtained data suggest a positive therapeutic effect of AAV2/9-GBA1 even considering that this therapeutic vector is of same serotype as the one used for disease modelling purposes. In other words, the obtained therapeutic effect is not affected by a previous injection of a vector virus with same serotype.

2. Studies in Non-Human Primates (NHPs) with rAAV2/9-GBA1:

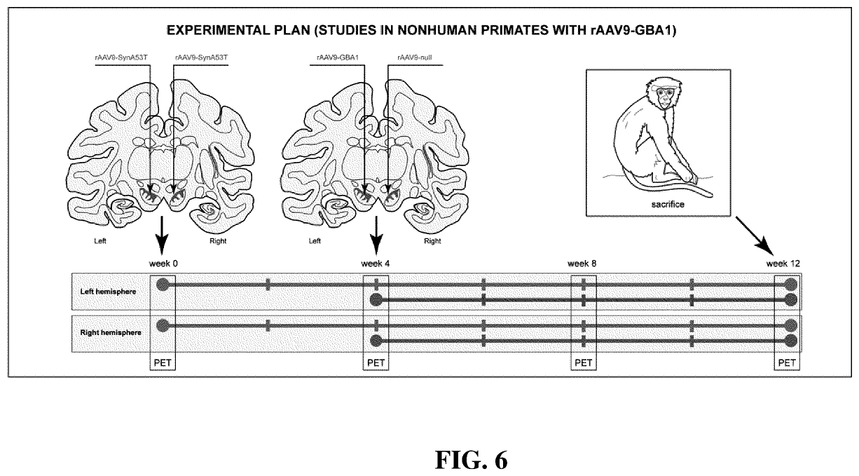

Wildtype non-human primates (Macaca fascicularis, n=4) were injected bilaterally into the caudal half SNc through stereotaxic surgery with a recombinant AAV serotype 9 coding for the mutated form of human alpha-synuclein under the control of a synapsin neuron specific promoter (rAAV2/9-SynA53T). Each SNc received two deposits of 5.0 microliters each of 1.36×10E13 of the viral suspension, spaced 2 mm in the rostrocaudal direction in an attempt to cover the whole extent of the caudal SNc. Once the neurodegenerative processes are already ongoing but before reaching a non-returning point (e.g. 4 weeks post-delivery of rAAV2/9-SynA53T), a recombinant AAV9 coding for the GBA1 gene under the control of a constitutive promoter GusB (rAAV2/9-GBA1) was delivered into the left SNc (1.0 microliters of 1.25×10E13 of the viral suspension), together with an empty, control rAAV9 coding for no transgene (r-AAV2/9-null) injected into the right SNc. 8 weeks later (e.g. 12 weeks after initial delivery of rAAV2/9-SynA53T), animals were sacrificed and processed for neuropathological analysis. The conducted experimental plan is summarized below (FIG. 6).

Neuroimage microPET Studies:

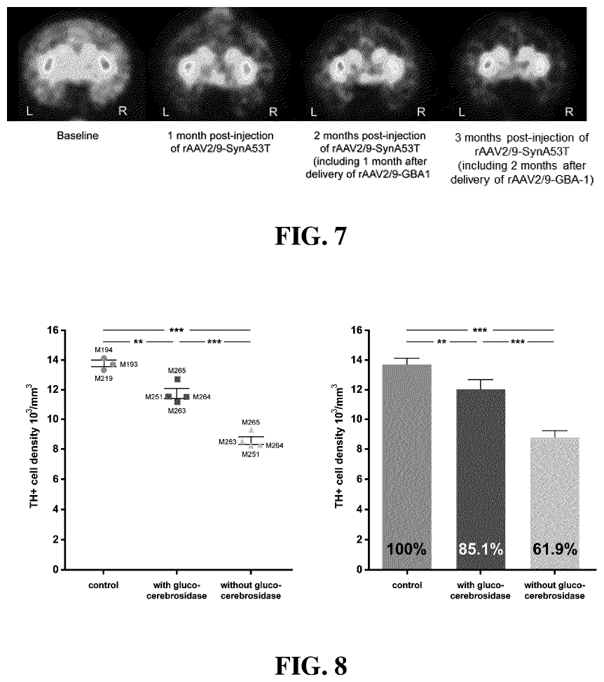

MicroPET studies were conducted in all animals at regular intervals, comprising one scan at baseline, followed by consecutive scans performed one, two and three months post-bilateral delivery of rAAV2/9-SynA53T into the SNc (e.g. one and two months post-injection of rAAV2/9-GBA1 and rAAv2/9-null viral vectors). The radiotracer chosen for these experiments was 11c-dihydrotetrabenazine, a selective VMAT2 ligand that allows the acquisition of sharp images with high reproducibility. Obtained results showed that the radiotracer binding potential measured into the left post-commissural putamen is higher than the one observed into the right post-commissural putamen (as shown in FIG. 7).

Neuropathological Data:

The neuropathological analysis conducted revealed that (1) the rAAV2/9-GBA1 mediated enhancement of GCase reduced alpha-synuclein burden and exerts a significant neuroprotection of dopaminergic (TH+) neurons in the SNc, together with a preserved innervation of the caudal post-commissural putamen located ipsilaterally to the left SNc injected with the rAAV2/9-GBA1, as shown from coronal sections of the NHP brain taken at the level of the post-commissural putamen and caudate nuclei and the mesencephalon, with immunohistochemical stains for tyrosine hydroxylase (Data not shown). (2) the unbiased stereological estimation of the density of tyrosine-positive neurons in the SNc showed an statistically very significant difference when comparing left vs. right SNc. Upon a follow-up of 12 weeks post-delivery of rAAV2/9-SynA53T into the right SNc, roughly a 39% of neuronal death was observed, contrasting with a 15% of neuronal death being noticed in the left SNc, i.e. the one treated with rAAV2/9-GBA1 (FIG. 8). (3) A prion-like transneuronal spread of mutated alpha-synuclein was observed into several brain areas of the right frontal cortex, where a moderate amount of pyramidal neurons expressing alpha-synuclein were noticed after 3 months post-delivery of rAAV2/9-SynA53T. Most importantly, AAV-mediated enhancement of GCase activity into the left SNc reduced alpha-synuclein burden, thus impeding the transneuronal spread of alpha-synuclein. In particular, immunohistochemical detection of alpha-synuclein show that 3 months of AAV-mediated over-expression of mutated alpha-synuclein resulted in the transeuronal spread of alpha-synuclein aggregates. Upon delivery of rAAV2/9-SynA53T into the right SNc, a high density of synuclein-positive fibers was observed coursing through the medial forebrain bundle, leading to the appearance of a moderate number of pyramidal neurons showing alpha-synuclein immunoreactivity throughout several brain areas of the frontal cortex. By contrast, after two months of AAV-mediated enhancement of GCase activity into the left SNc, the density of synuclein-positive fibers observed travelling through the medial forebrain bundle was clearly reduced, and indeed second-order labeled neurons in the frontal cortex have never been noticed (Data not shown). (4) Microglial-driven pro-inflammatory phenomena were attenuated in the left SNc, i.e. the one treated with rAAV2/9-GBA1 (Data not shown). Analysis of microglial phenotypes by comparing treated vs. untreated sides of the SNc has been carried out using antibodies against the major histocompatibility complex class II (MHC-II), a specific marker for activated microglial phenotypes (e.g. resting microglial cells lack expression of MHC-II).

As mentioned for the mice studies, non-human primate data suggest a positive therapeutic effect of AAV2/9-GBA1 even considering that this therapeutic vector is of same serotype as the one used for disease modelling purposes. In other words, the obtained therapeutic effect is not affected by a previous injection of a vector virus with same serotype.

3. Retrograde Dissemination of GCase Resulted in Clearance of Phosphorylated Alpha-Synuclein Throughout the Cerebral Cortex in Mice

It has been postulated that alpha-synuclein might progress through brain circuits in a prion-like manner. This phenomenon explains the clinical course of the disease, initially characterized by motor symptoms as a result of alpha-synuclein aggregates located into the SNc, later progressing to non-motor symptoms (dementia and neuropsychiatric symptoms) as a result of the presence of a progressive synucleinopathy engaging brain areas other than the SNc, particularly the cerebral cortex. In this regard, it is worth noting that this progressive synucleinopathy, when affecting the cerebral cortex is the main driving force sustaining the appearance of dementia and neuropsychiatric symptoms in advanced stages of PD and therefore when coming to design disease-modifying therapeutic alternatives, finding a way for accurately targeting such a widespread synucleinopathy would be key. In other words, bearing in mind that PD patients in advanced disease stages will exhibit a generalized synucleinopathy throughout a number of brain areas—particularly the cerebral cortex, among others- and there is still a need to new therapeutic avenues for a product with broad dissemination throughout the brain areas to be treated.

Quite the same also applies when considering synucleinopathies other than PD such as DLB, where the main neuropathological hallmark is represented by the presence of Lewy bodies and Lewy neurites across the cerebral cortex.

Overall, at present the main unmet medical need is to develop disease-modifying strategies for PD and related synucleinopathies, intended to slow-down—or even ideally arrest—the unrelenting progressive course of these devastating brain disorders. Any successful therapeutic approach should therefore preferably be efficient in conducting alpha-synuclein clearance throughout the brain, particularly when facing PD patients in advanced disease stages, these patients being the most likely ones to benefit from treatments intended to minimize disease progression rates.

The inventors therefore first tested whether AAV variant particles with retrograde transport would be able to disseminate efficiently from the injected area to distant area including in particular cortical areas.

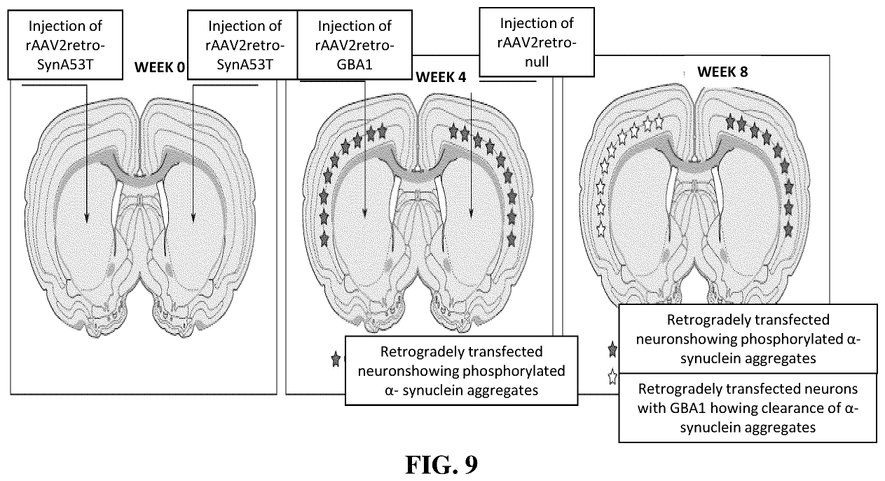

Wild type mice were injected bilaterally into the striatum through stereotaxic surgery with a recombinant AAV2-retro coding for the mutated form of human alpha-synuclein under the control of a synapsin neurospecific promoter (rAAV2retro-SynA53T). Each striata received 2.0 microliters of 3.71×10E12 vp/ml of the viral suspension, to further generate a widespread synucleinopathy throughout the cerebral cortical areas innervating the injected striata. Four weeks later, a recombinant AAV2-retro coding for the GBA1 gene under the control of a constitutive promoter GusB (rAAV2retro-GBA1) was delivered into the left striatum (2.0 microliters; 3.71×10E12 vp/ml of the viral suspension) together with an empty, control rAAV2-retro coding for no transgene (rAAV2retro-null) injected into the right striatum. Four weeks later (e.g. eight weeks after initial delivery of rAAV2retro-SynA53T), animals were sacrificed and processed for neuropathological analysis. The conducted experimental plan is summarized below (FIG. 9).

Neuropathological Data:

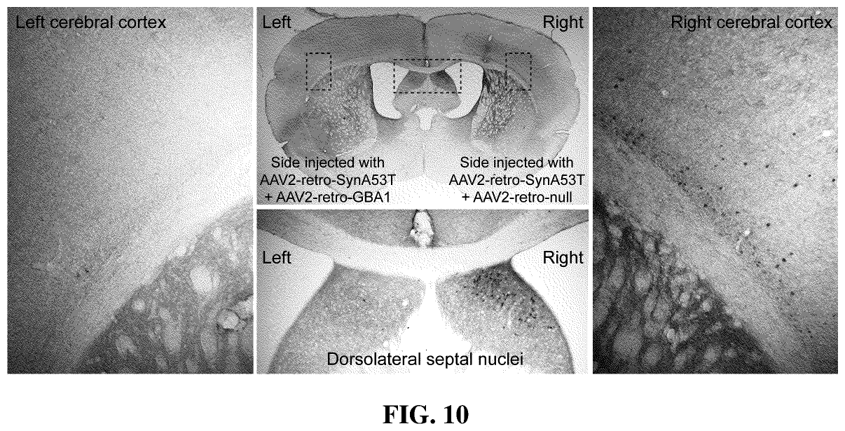

The conducted analysis revealed that the AAV2retro-GBA1-mediated enhancement of GCase resulted in an almost complete clearance of phosphorylated alpha-synuclein throughout the cerebral cortex at the level of the left brain hemisphere (e.g. the one located ipsilaterally to the striatum firstly injected with rAAV2retro-SynA53T and later on with rAAV2retro-GBA1). By contrast, an evident synucleinopathy still persists within a number of cortical areas of the right brain hemisphere (e.g. the one located ipsilaterally to the striatum firstly injected with rAAV2retro-SynA53T and later on with the control vector rAAV2retro-null) (FIG. 10).

4. Studies in Nonhuman Primates (NHPs) with rAAV-TT-GBA1:

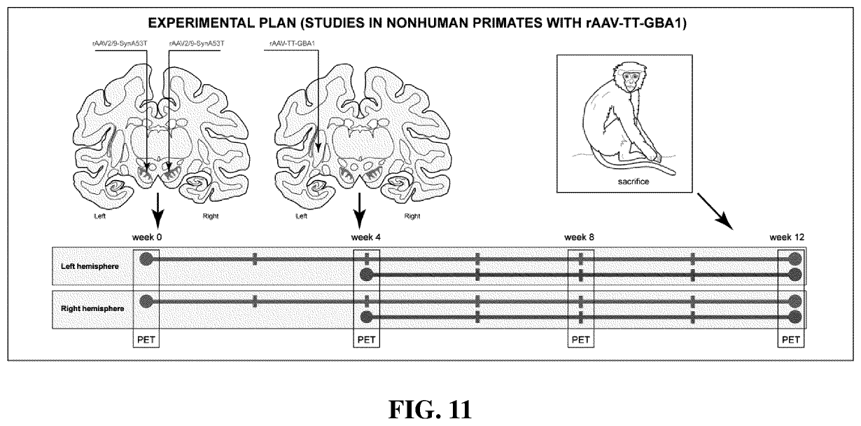

Wildtype nonhuman primates (Macaca fascicularis, n=4) were injected bilaterally into the caudal half SNc through stereotaxic surgery with a recombinant AAV serotype 9 coding for the mutated form of human alpha-synuclein under the control of a synapsin neuron specific promoter (rAAV2/9-SynA53T). Each SNc received two deposits of 5.0 microliters each of 1.36×10E13 of the viral suspension, spaced 2 mm in the rostrocaudal direction in an attempt to cover the whole extent of the caudal SNc. Once the neurodegenerative processes are already ongoing but before reaching a non-returning point (e.g. 4 weeks post-delivery of rAAV2/9-SynA53T), a recombinant AAV-TT coding for the GBA1 gene under the control of a constitutive promoter CAG (rAAV-TT-GBA1) was delivered into the left post-commissural putamen (2×10 microliters of 1×10E13 of the viral suspension). Pressure-injections were achieved in pulses of 0.5 μL/min. In non-human primate the debit is adjusted to the lower range. However, in human trials high injection speed allows virus stability, and better patient management and debit can range from 0.5 μL to 5 μL/min. 8 weeks later (e.g. 12 weeks after initial delivery of rAAV2/9-SynA53T), animals were sacrificed and processed for neuropathological analysis. The conducted experimental plan is summarized below (FIG. 11).

Neuroimage microPET Studies:

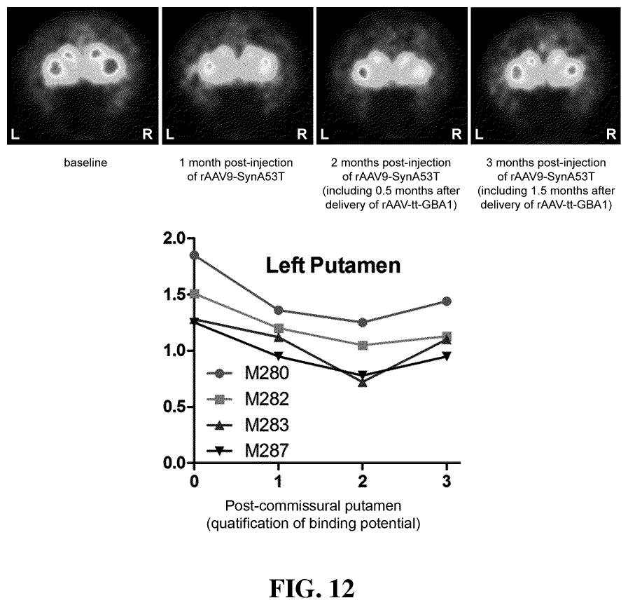

MicroPET studies were conducted in all animals at regular intervals, comprising one scan at baseline, followed by consecutive scans performed one, two and three months post-delivery of rAAV2/9-SynA53T (e.g. one and two months post-injection of rAAV-TT-GBA1 viral vector). The radiotracer chosen for these experiments was 11c-dihydrotetrabenazine (11C-DTBZ), a selective VMAT2 ligand that allows the acquisition of sharp images with high reproducibility. Obtained results showed that the radiotracer binding potential measured into the left post-commissural putamen was 24.44% higher than the one observed 1.5 months before the delivery of rAAV-TT-GBA1. Obtained results are illustrated below in FIG. 12.

Neuropathological Data:

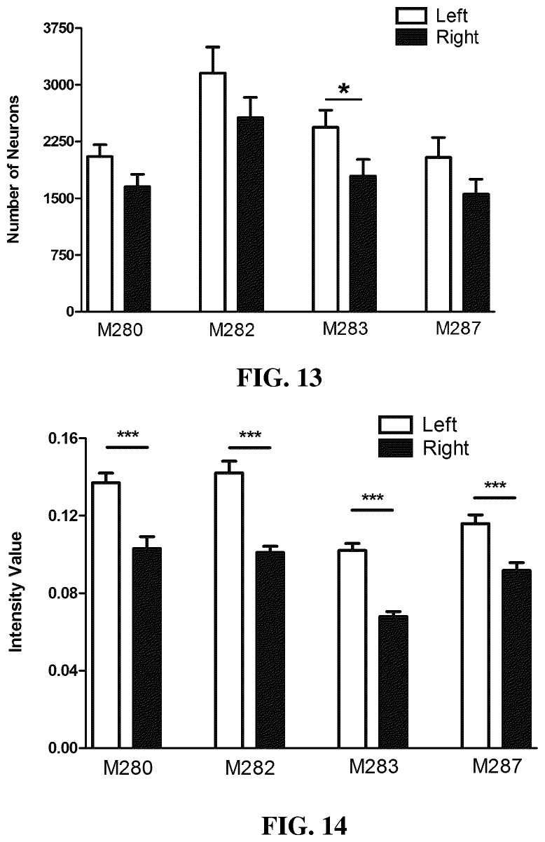

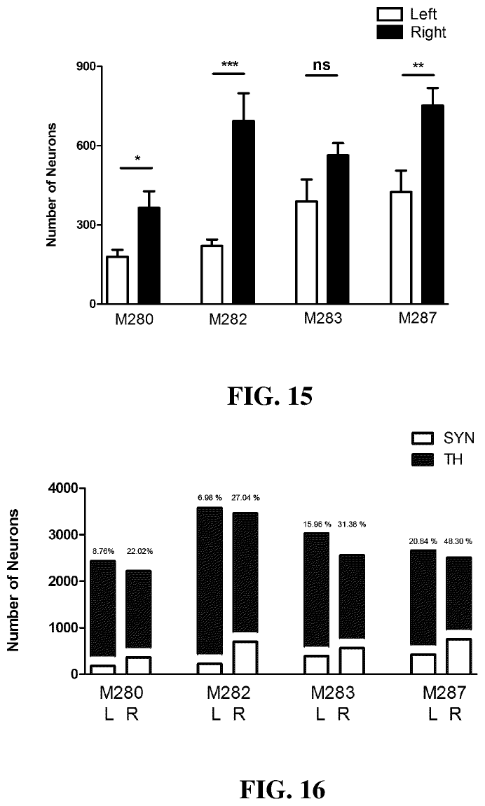

The conducted neuropathological analysis revealed that (1) the rAAV-TT-GBA1 mediated enhancement of GCase reduced alpha-synuclein burden and exerts a significant neuroprotection of dopaminergic (TH+) neurons in the SNc, together with a preserved innervation of the caudal post-commissural putamen located ipsilaterally to the left SNc injected with the rAAV2/9-GBA1, as shown from coronal sections of the NHP brain taken at the level of the post-commissural putamen and caudate nuclei and the mesencephalon, with immunohistochemical stains for tyrosine hydroxylase (Data not shown). (2) the automated counting of the number of tyrosine-positive neurons in the SNc showed a statistically very significant difference when comparing left vs. right SNc. Upon a follow-up of 12 weeks post-delivery of rAAV2/9-SynA53T into the left and right SNc (e.g. two months post-delivery of rAAV-TT-GBA1 into the left post-commissural putamen), the total number of tyrosine-positive neurons in the left SNc was found to be 22.3% higher than the number of tyrosine-positive neurons in the right SNc (e.g. the one not treated with the intraputaminal delivery of rAAV-TT-GBA1) (FIG. 13). The automatic measuring of the optical density (OD) for TH stain into the left and right post-commissural putamen of 4 NHPs injected with rAAV-TT-GBA1 into the left post-commissural putamen showed that the mean OD was 27.42% lower in the right vs. left post-commissural putamen (the latter being the one treated with the intraputaminal delivery of rAAV-TT-GBA1) (FIG. 14). (3) Furthermore, the automated counting of the number of alpha-synuclein-positive neurons in the SNc showed a statistically significant difference when comparing left vs. right SNc. Upon a follow-up of 9 weeks post-delivery of rAAV2/9-SynA53T into the left and right SNc (e.g. 1.5 months post-delivery of rAAV-TT-GBA1 into the left post-commissural putamen), the total number of alpha-synuclein-expressing neurons in the left SNc was found to be 48.33% lower than the number of alpha-synuclein-positive neurons in the right SNc (e.g. the one not treated with the intraputaminal delivery of rAAV-TT-GBA1) (FIG. 15).

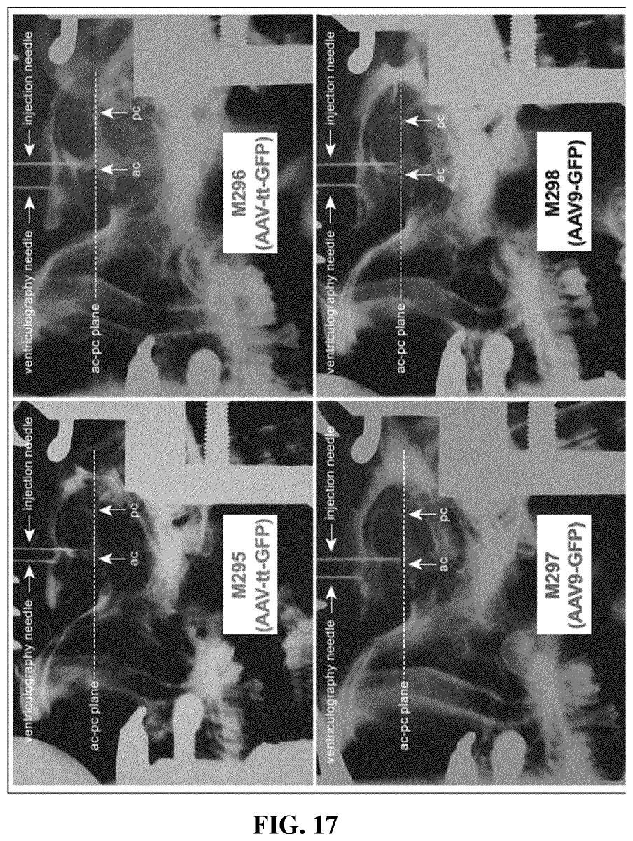

The percentage of alpha-synuclein expressing neurons are consistently lower in the left SNc (e.g. the one located ipsilaterally to the post-commissural putamen being injected with AAV-TT-GBA1) than in the right SNc (untreated side) (FIG. 16).

As mentioned for the mice studies, non-human primate data suggest a positive therapeutic effect of rAAV-TT-GBA1.

5. Biodistribution and Comparative Performance of AAV-TT-GFP and AAV9-GFP in the Nonhuman Primate Brain

5.1 Conducted Experiments

Up to four adult juvenile male Macaca fascicularis primates (body weight between 3.0 to 3.4 Kg) were injected with either 5 μL of AAV-TT-GFP (1×10¹³ vg/mL; 2 animals) or with 5 μl of AAV9-GFP (1×10¹³ vg/mL; 2 animals). Both AAVs were coding for GFP under the control of a CAG promoter.

AAVs were administered through ventriculography-assisted stereotaxic surgery by taking advantage of a Hamilton syringe. Pressure-injections were achieved in pulses of 0.5 μL/min. In non-human primate the debit is adjusted to the lower range. However, in human trials high injection speed allows virus stability, and better patient management and debit can range from 0.5 μL to 5 μL/min. Once AAV delivery was completed, the injection needle was left in place for additional 10 min to minimize AAV reflux through the injection tract (FIG. 17). Just before surgery, body fluid samples (blood and CSF) were collected and stored at −80° C.

Animals were sacrificed one-month post-AAV delivery through intracardiac perfusion. Before sacrifice, body fluid samples (blood and CSF) were collected and stored at −80° C. The perfusates consisted of a saline Ringer solution followed by a buffered solution of paraformaldehyde (3,000 ml/animal) and by 1,000 ml of a cryoprotective solution made of 10% glycerin and 1% DMSO in phosphate buffer 0.1 M, pH 7.3.

During perfusion with the Ringer solution, fresh tissue samples (e.g. unfixed) were taken from a number of peripheral organs, these including heart, lung, liver, spleen, pancreas, kidney, testis and striatal muscle. Samples were frozen on dry ice and stored at −80° C.

Once perfusion is completed, the brain was removed from the skull and brain blocks of approximately 1 cm wide were made and stored in a cryoprotective solution made of 20% glycerin and 2% DMSO in phosphate buffer 0.1 M, pH 7.3 (pia matter removed from all brain blocks). Samples from fixed peripheral organs were obtained (heart, lung, liver, spleen, pancreas, kidney, testis, retroperitoneal ganglia, pineal gland and striatal muscle) and further embedded in paraffin.

After a minimum of 48 h in the cryoprotective solution, 10 series of frozen coronal brain sections (40 μm-thick) were made in a sliding microtome and collected in the cryoprotective solution. One entire series of sections (e.g. comprising every 10th section of the monkey brain) was processed for the immunoperoxidase detection of GFP by taking advantage of a primary polyclonal antibody raised in rabbit. Upon incubation with a biotinylated goat anti-rabbit IgG, sections were then incubated with an ABC kit and finally stained using H2O2-DAB solution. Once stained is completed, free-floating sections were mounted on microscopy slides, air-dried overnight and cover-slipped with entellan. Stained sections were scanned using an Aperio CS2 slide scanner (Leica) and processed using dedicated software.

5.2 Results

Labeling with either AAV-TT-GFP or with AAV9-GFP was only found throughout brain territories known to innervate the post-commissural putamen, whereas not even a single labeled neuron was observed in brain territories not innervating the injection site (e.g. the hippocampus, cerebellum, etc). Moreover, obtained retrograde labeling was of “Golgi-like” morphology, i.e. neuronal labeling was not limited to cell somata and indeed extends over distal dendrites, particularly in locations throughout the cerebral cortex. It is also worth noting that small dendritic processes such as dendritic spines are sometimes even visible.

Events at the Injection Site

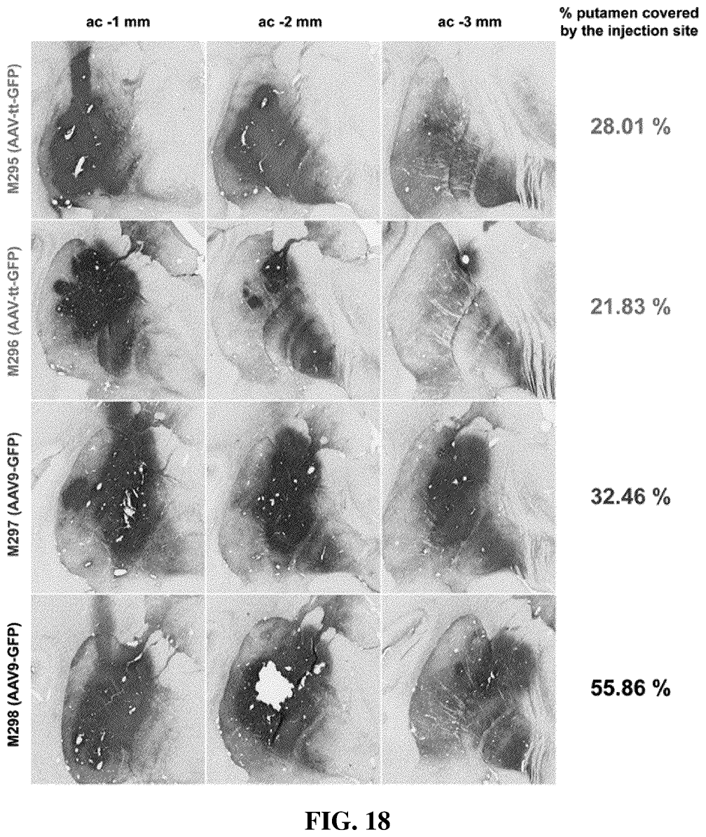

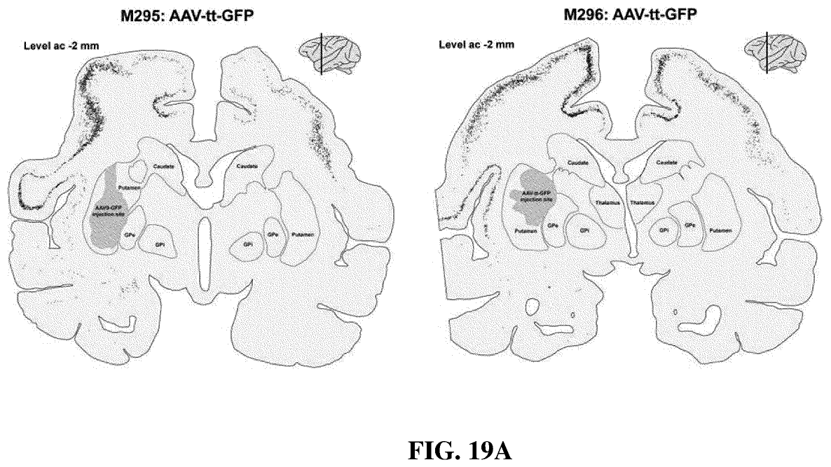

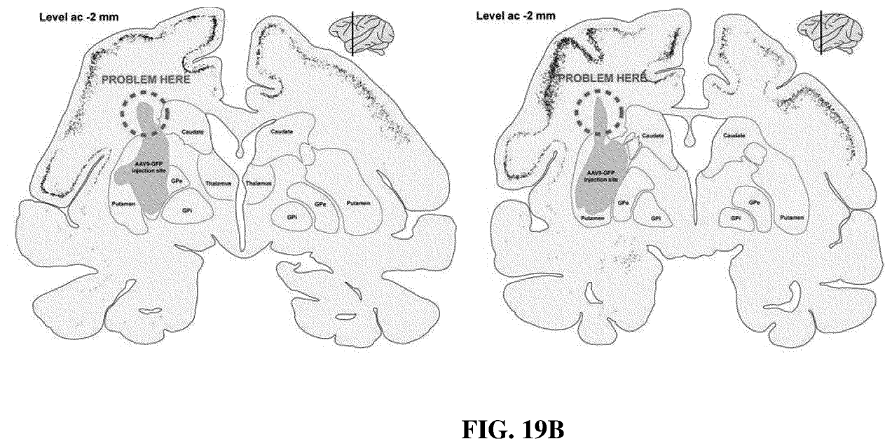

Both injections of AAV-TT-GFP were accurate and properly located within the boundaries of the post-commissural putamen. Obtained sizes for the injection sites were consistently smaller for AAV-TT-GFP than for AAV9-GFP, covering 28.01% and 21.83% in animals M295 and M296 (injected with AAV-TT-GFP), whereas in animals injected with AAV9-GFP (M297 and M298), 32.46% and 55.86% of the post-commissural putamen was comprised within the injection sites, respectively (FIG. 18). Both injections with AAV-TT-GFP showed a completely lack of AAV uptake through the injection tract (e.g. these are both very clean injections). By contrast, injections performed with AAV9-GFP exhibited a moderate-to-high uptake through the injection tract, meaning that it is very likely that obtained results are contaminated by false positive labeling (probably particularly notorious within cortical territories) due to AAV9-GFP uptake by white matter tracts located above the post-commissural putamen. Troubles related to false positive results are illustrated in FIG. 19. Furthermore, the delivery of AAV9-GFP in animal M297 has spread beyond the boundaries of the post-commissural putamen, and also includes a substantial part of the external globus pallidus (GPe).

The Potency of Retrograde Spread

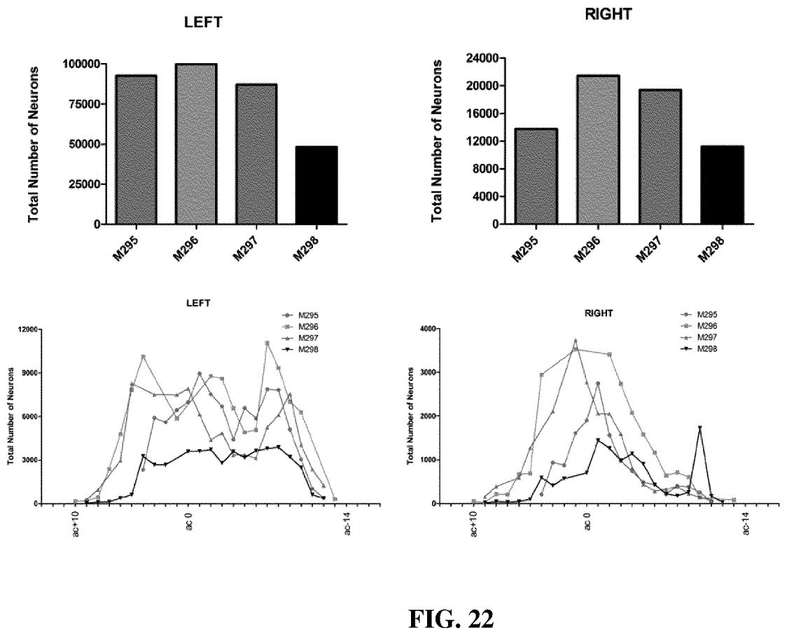

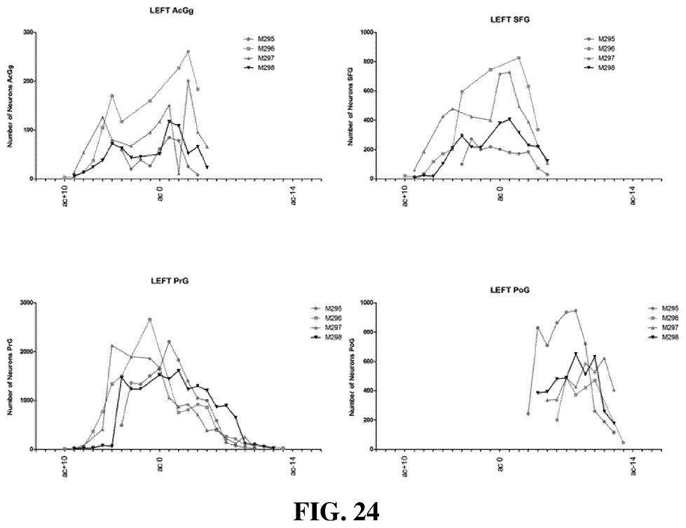

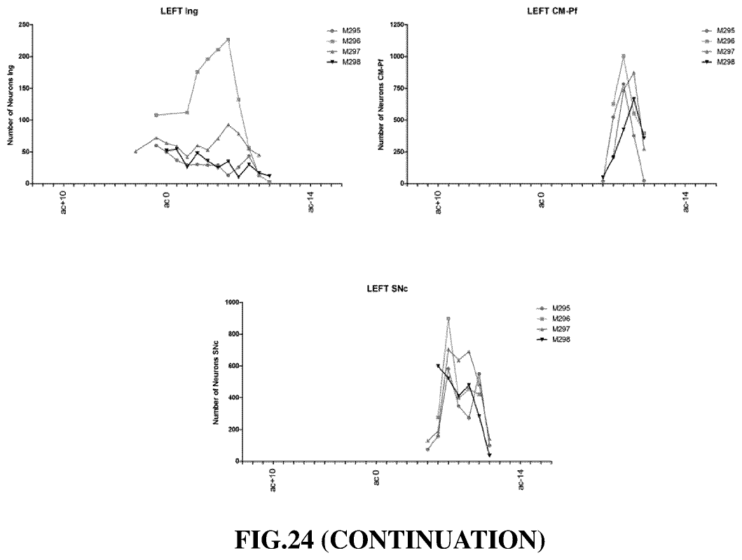

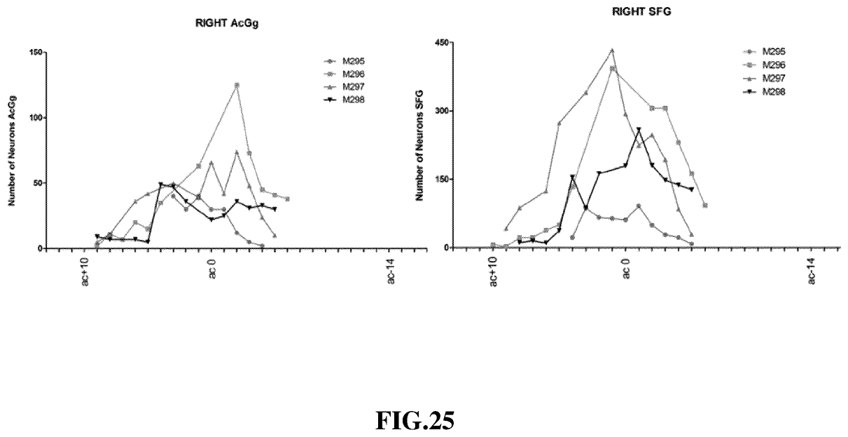

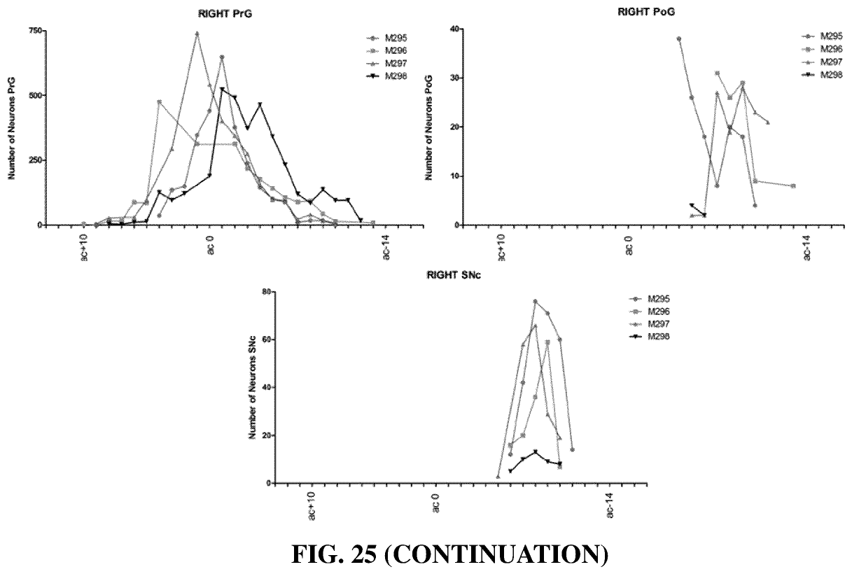

Both the total numbers and observed intensities of retrogradely-labeled neurons are directly related to the extent of the injection sites. In other words, higher numbers of GFP+ neurons are expected from injection sites covering larger territories of the post-commissural putamen. In this regard, and besides providing an accurate quantification of the number of neurons observed in each region of interest, this final number needs to be corrected by the extent of the post-commissural putamen area being covered by the injection site. Numbers of neurons are provided based on the quantification done with Aiforia® (labeled as “raw” and “corrected” data). In an attempt to properly compare the performance of AAV-TT vs. AAV9, obtained raw data need to be standardized by taking into consideration the extent of the injection site. Accordingly, a correction factor based on the size of the injection site was calculated to properly estimate the expected retrograde spread of each AAV. Correction factors were ×3.57 for M295, ×4.58 for M296, ×3.08 for M297 and ×1.79 for M298. Correction factors were used for generating data showed in FIGS. 20-25.

Brain Areas Showing Strongest Labeling

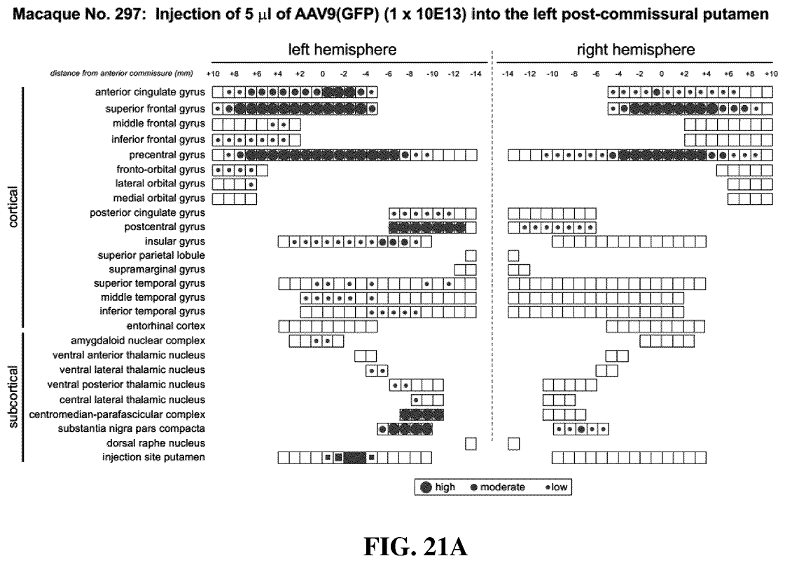

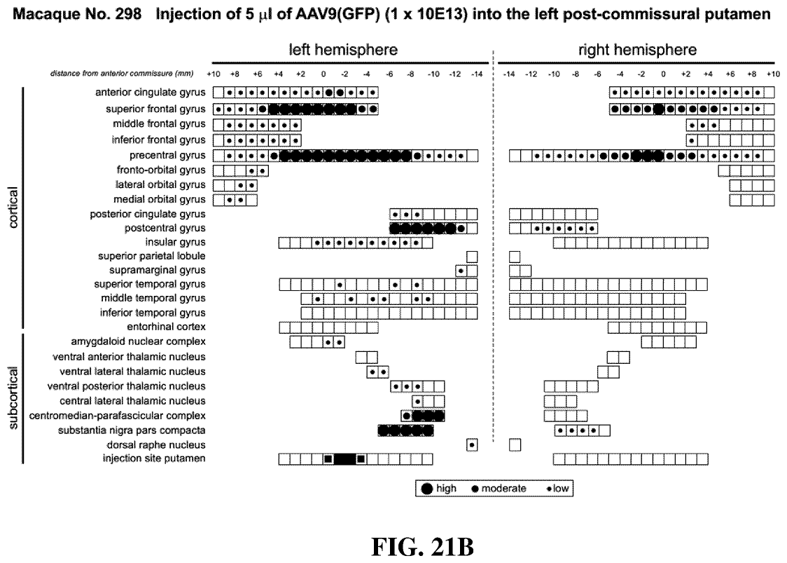

In all animals (AAV-TT-GFP and AAV9-GFP), the strongest labeling was observed in the superior frontal gyrus and in the precentral gyms (FIG. 20-21). Other cortical areas consistently showing GFP+ neurons (although to a lower extent) are the anterior cingulate cortex, the postcentral gyrus and the insular gyrus. Sparse neuronal labeling was observed in the middle frontal gyms, inferior frontal gyrus, orbital frontal cortex (frontal orbital, lateral orbital and medial orbital territories), superior, middle and inferior temporal gyri, as well as in the superior parietal lobule and the supramarginal gyrus. Furthermore, GFP+ neurons were consistently found in the contralateral cortex as mirror-like representations of the ipsilateral cortex (obviously containing a much lower number of GFP+ neurons). Results are fully consistent with what was expected and indeed very relevant, bearing in mind that upon AAV delivery in the post-commissural putamen (the motor-related putamenal territories), the strongest labeling was observed in both the precentral and superior frontal gyri (cortical gyri containing the primary motor cortex and the supplementary motor area, respectively). Regarding subcortical labeling, two structures are particularly relevant, namely the substantia nigra pars compacta (SNc) and the centromedian-parafascicularis complex (CM-Pf).

Moreover, the amount of GFP+ neurons observed in the CM-Pf is very impressive, although expected, bearing in mind that the CM-Pf thalamic complex is the main source of thalamostriatal projections. Besides CM-Pf, sparse labeling was also found in the ventral anterior, ventral lateral and ventral posteromedial thalamic nuclei, centrolateral and paracentral intralaminar nuclei and the dorsal raphe nucleus (a small brainstem nucleus known to be the main source of serotoninergic projections to the putamen). Furthermore, observed labeling at the level of the amygdaloid complex is lower than initially expected for both AAV types. Although the amygdaloid complex has often been viewed as another source of afferents to the putamen (together with the cortex, thalamus and substantia nigra), data obtained with AAV-TT and with AAV9 clearly suggested that the importance of this anatomical pathway has been likely overestimated in earlier anatomical studies.

Striatal Afferent Systems

Although the present study was not designed for this purpose, the conducted quantification allows to numerically estimate the “weight” of each different striatal afferent system, namely the corticostriatal pathways (ipsi- and contralateral), thalamostriatal and nigrostriatal projections. Obtained data showed that ipsilateral corticostriatal projections are by far the most abundant ones (69.37% of total striatal afferents on average), followed by contralateral corticostriatal-projecting neurons (15.99% of total striatal afferents), then nigrostriatal projections (7.99% on average) and finally the thalamostriatal projections arising from the centromedian-parafascicular thalamic complex (6.67%). In this regard, it is also worth noting that although the contralateral corticostriatal pathway has often been neglected in most studies dealing with basal ganglia function and dysfunction, this projection roughly represents up to 16% of total striatal afferents, a percentage clearly above the ones related to thalamostriatal and nigrostriatal projections.

Obtained results supported a superior performance of AAV-TT-GFP when compared to AAV9-GFP. A deep comparison of the results showed that AAV-TT is a better candidate than AAV9. AAV-TT holds some important advantages, particularly when dealing with a higher “potency” in terms of retrograde spread, together with the lack of uptake through the injection tract. The use of AAV-TT presents a complete lack of false positives.

6. Comparison of the Performance of AAV-TT-GBA1 vs. AAV9-GBA1

6.1 Conducted Experiments

Wildtype nonhuman primates (Macaca fascicularis, n=8) (body weight between 4.3 to 10.4 kg) were injected into the left and right substantia nigra pars compacta through stereotaxic surgery with AAV2/9-SynA53T. Each SNc received two deposits of 5.0 microliters each of 1.26×10E12 vg/mL of the viral suspension, spaced 1 mm in the rostrocaudal direction in an attempt to cover the whole extent of the caudal SNc (e.g. total of 4 injections/animal).

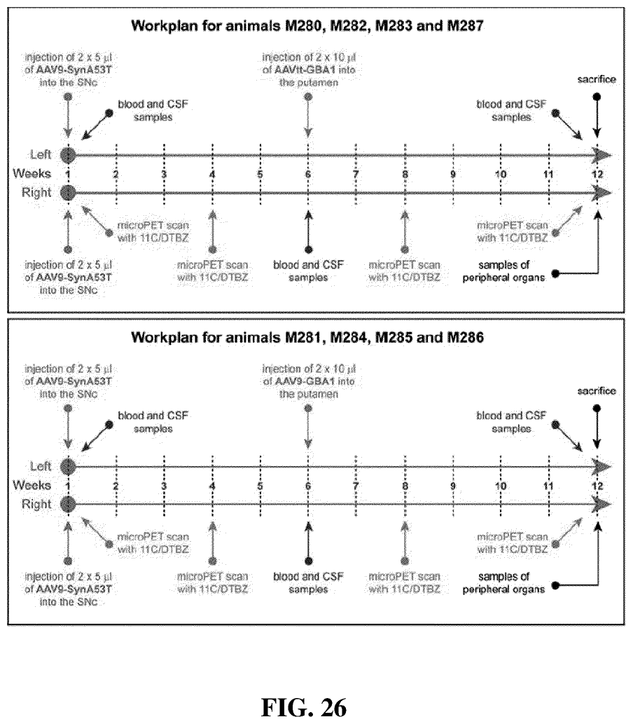

6 weeks post-delivery of AAV2/9-SynA53T, a recombinant AAV-TT coding for the GBA1 gene under the control of a constitutive promoter CAG (AAV-TT-GBA1) was delivered into the left post-commissural putamen (2×10 microliters of 1×10E13 vg/mL of the viral suspension; 4 animals, injections spaced 1 mm from each other in the rostrocaudal direction). 8 weeks later (e.g. 12 weeks after initial delivery of AAV2/9-SynA53T), animals were sacrificed and processed for neuropathological analysis. The conducted experimental plan is summarized in FIG. 26. AAVs were administered through ventriculography-assisted stereotaxic surgery by taking advantage of a Hamilton syringe. Pressure-injections were achieved in pulses of 0.5 μL/min. In non-human primate the debit is adjusted to the lower range. However, in human trials high injection speed allows virus stability, and better patient management and debit can range from 0.5 μL to 5 μL/min. Once AAV delivery was completed, the injection needle was left in place for additional 10 min to minimize AAV reflux through the injection tract.

Body fluid samples (blood and CSF) were collected at baseline (e.g. before surgeries with AAV9-SynA53T), prior to deliveries of GBA1-coding vectors and at sacrifice.

MicroPET scans with the radiotracer 11C-dihydrotetrabenazine (11C-DTBZ) were performed at baseline and 4, 8 and 12 weeks post-injection of AAV9-SynA53T.

Animals were sacrificed 12 weeks post-delivery of AAV9-SynA53T (e.g. 6 weeks post-injection of either AAV-tt-GBA1 or AAV9-GBA1) through intracardiac perfusion. Before sacrifice, body fluid samples (blood and CSF) were collected and stored at −80° C. The perfusates consisted of a saline Ringer solution followed by a buffered solution of paraformaldehyde (3,000 ml/animal) and by 1,000 ml of a cryoprotective solution made of 10% glycerin and 1% DMSO in phosphate buffer 0.1 M, pH 7.3.

During perfusion with the Ringer solution, fresh tissue samples (e.g. unfixed) were taken from a number of peripheral organs, these including heart, lung, liver, spleen, pancreas, kidney, testis and striatal muscle. Samples were frozen on dry ice and stored at −80° C.

Once perfusion is completed, the brains were removed from the skull and brain blocks of approximately 1 cm wide were made and stored in a cryoprotective solution made of 20% glycerin and 2% DMSO in phosphate buffer 0.1 M, pH 7.3 (pia matter removed from all brain blocks). Samples from fixed peripheral organs were obtained (heart, lung, liver, spleen, pancreas, kidney, testis, retroperitoneal ganglia, pineal gland and striatal muscle) and further embedded in paraffin (sent to MOTAC).

After a minimum of 48 h in the cryoprotective solution, 10 series of frozen coronal brain sections (40 m-thick) were made in a sliding microtome and collected in the cryoprotective solution. One entire series of sections (e.g. comprising every 10th section of the monkey brain) was processed for the immunoperoxidase detection of tyrosine hydroxylase (TH) by taking advantage of a primary polyclonal antibody raised in goat. Upon incubation with a biotinylated donkey anti-goat IgG, sections were then incubated with an ABC kit and finally stained using H2O2-DAB solution. Furthermore, another entire series of sections was processed for the immunoperoxidase detection of alpha-synuclein (α-syn) by taking advantage of a primary monoclonal antibody raised in mouse. Upon incubation with a biotinylated donkey anti-mouse IgG, sections were then incubated with and ABC kit and finally stained using H2O2-DAB solution. Once stains are completed, free-floating sections were mounted on microscopy slides, air-dried overnight and coverslipped with entellan. Stained sections were scanned using an Aperio CS2 slide scanner (Leica) and processed using dedicated software.

6.2 Results

The performance of AAV-TT-GBA1 vs. AAV9-GBA1 for alpha-synuclein clearance as well as the potential neuroprotective effect of GCase enhancement and alpha-synuclein removal in nigrostriatal-projecting dopaminergic neurons was compared.

Neuroimage microPET Studies

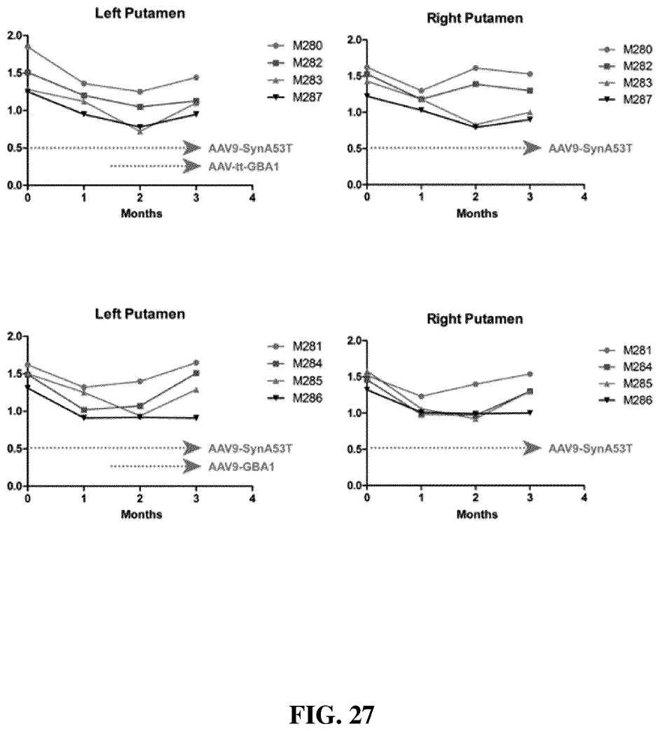

MicroPET scans with 11C-DTBZ were performed at baseline, and 4, 8 and 12 weeks post-injection of AAV9-SynA53T. Values for radiotracer binding potential were calculated through regions of interest comprising the entire rostrocaudal extent of the post-commissural putamen (typically engaging between 5 and 9 different sections) (FIG. 27).

In animals injected with AAV-TT-GBA1, there is a mild improvement in radiotracer binding at 6 weeks post-delivery of AAV-TT-GBA1 (e.g. 12 weeks post-injection of AAV9-SynA53T) compared to binding values at 2 weeks of AAV-TT-GBA1 (e.g. just 6 weeks after injection of AAV9-SynA53T). Observed increases are of 1% (M282), 15% (M280), 22% (M287) and 53% (M283). When compared to the binding values at baseline (e.g. prior to the induction of synucleinopathy), obtained values for all animals remained below baseline levels (−22% in M280, −24% in M287, −25% in M282 and −32% in M283). Obtained values for the right post-commissural putamen (non-injected side) remained roughly similar to baseline for animals M280 and M282, whereas a moderate decline was observed in animals M283 and M287.

Regarding animals injected with AAV9-GBA1, highest levels of radiotracer uptake were noticed in animals M281, M284 and M285, without any apparent effect on animal M286. Values for M281 are not surprising, bearing in mind that this animal was not appropriately injected with AAV9-SynA53T (mistargeted left and right substantia nigra). Although M281, M284 and M285 finally reached roughly similar levels at the end of the follow-up period compared to baseline, it is also worth noting that similar increases were also observed in the right putamen for the same animals.

Nigrostriatal Innervation (Optical Densities)

When explaining why dopaminergic neurons of the substantia nigra pars compacta are so vulnerable to die in the context of Parkinson's disease, it has been hypothesized that unique architecture of large and complex axonal arborizations of nigrostriatal projections (Matsuda et al., J Neurosci. 2009 Jan. 14; 29(2): 444-453) puts dopaminergic neurons under such a high energy budget that it makes them particularly susceptible to factors that contribute to cell death (Bolam and Pissadaki, Mov Disord. 2012 October; 27(12):1478-83). Indeed, it is worth noting that although the volume of the striatum has increased by approximately 300-fold from rats (19.9 mm³) to humans (6,280 mm³), the number of dopaminergic neurons has increased by only 32-fold. In summary, when dealing with neuroprotective studies, to what extent the nigrostriatal pathway was preserved deserves strong attention.

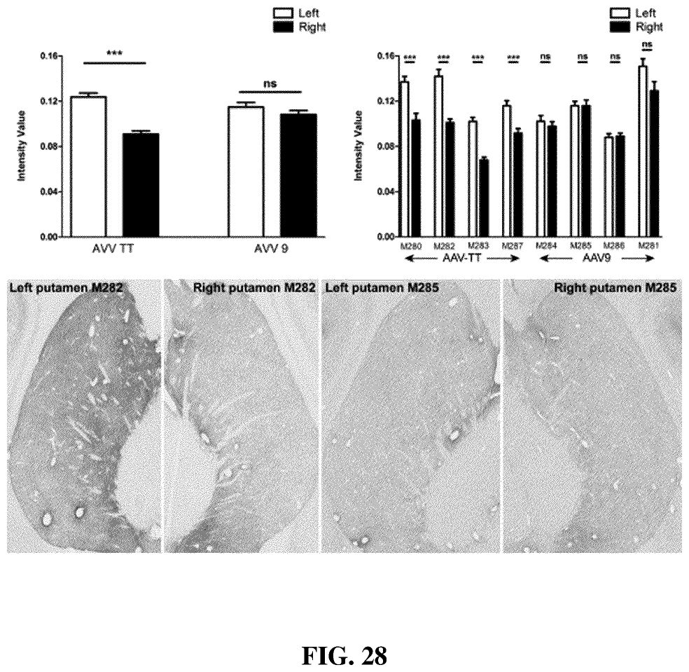

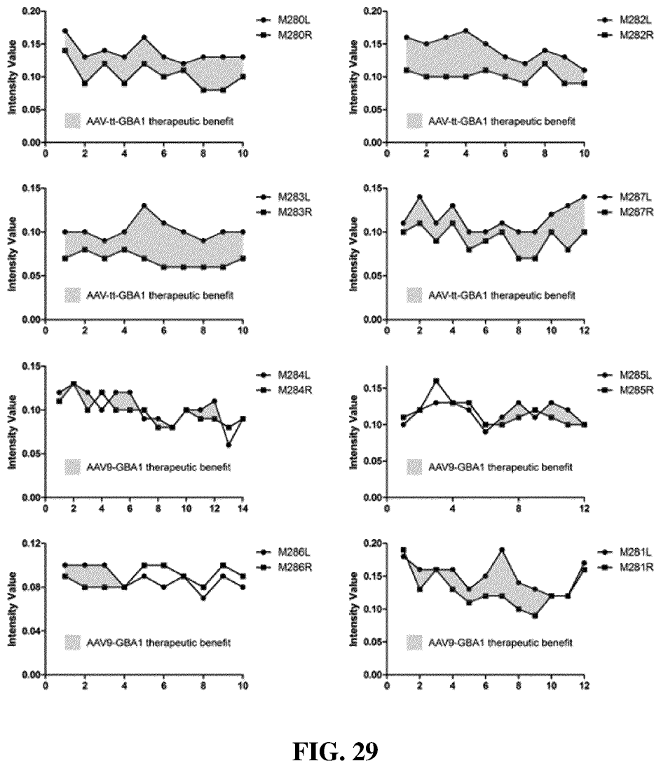

For these purposes, 10 to 12 equally-spaced rostrocaudal coronal sections covering the whole extent of the post-commissural putamen were stained for TH and further analyzed for optical density (OD). Obtained data comparing OD values for left and right post-commissural putamen levels are illustrated in FIGS. 28 and 29.

Left vs. right OD differences reflecting therapeutic benefit were consistently found to be higher throughout the entire rostrocaudal extent of the post-commissural putamen in animals injected with AAV-TT-GBA1 than in animals injected with AAV9-GBA1.

Neuroprotective Effect of GBA1-Coding AAVs for Dopaminergic Neurons

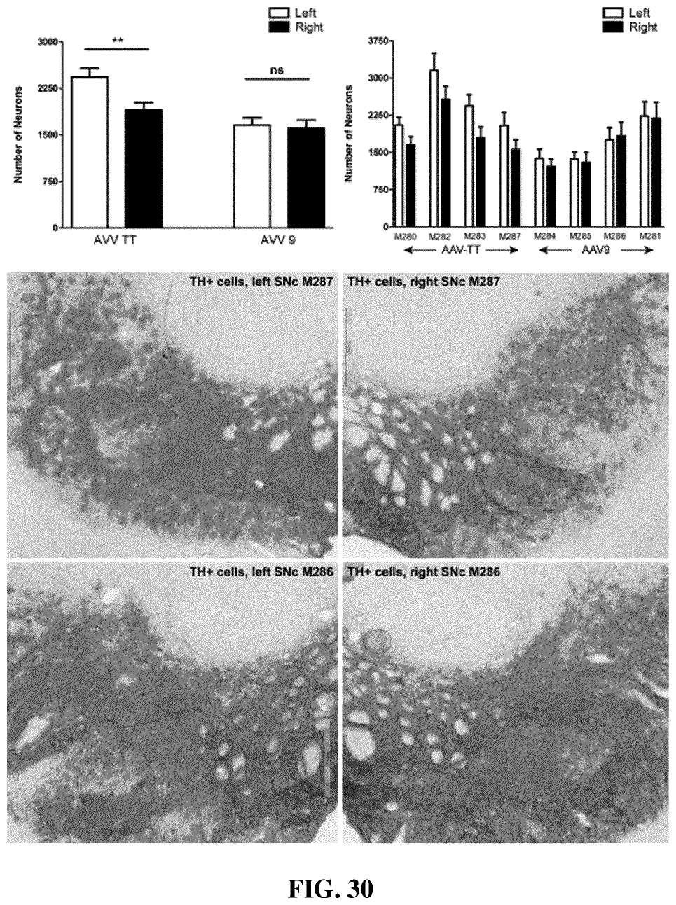

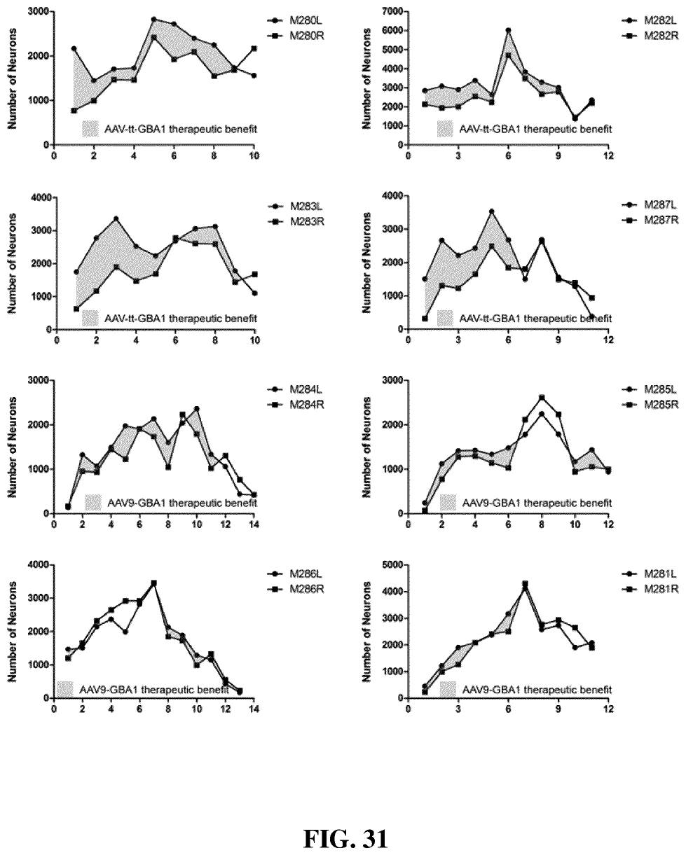

One of the main aims of the conducted studies was to evaluate to what extent AAV-driven GCase enhancement might exert a neuroprotective effect of nigrostriatal-projecting, dopaminergic neurons. For this purpose, up to 14 coronal sections stained for TH and covering the whole rostrocaudal extent of the substantia nigra were analyzed for dopaminergic cell counting. Analysis was conducted using the deep learning algorithm Aiforia®. Obtained data are illustrated in FIGS. 30 and 31.

Left vs. right differences reflecting therapeutic benefit were consistently found to be higher in animals injected with AAV-TT-GBA1 than in animals injected with AAV9-GBA1. Indeed, the therapeutic benefit of AAV-TT-GBA1 is maintained through most of the rostrocaudal extent of the substantia nigra.

Effect of GBA1-Coding AAVs for Alpha-Synuclein Clearance

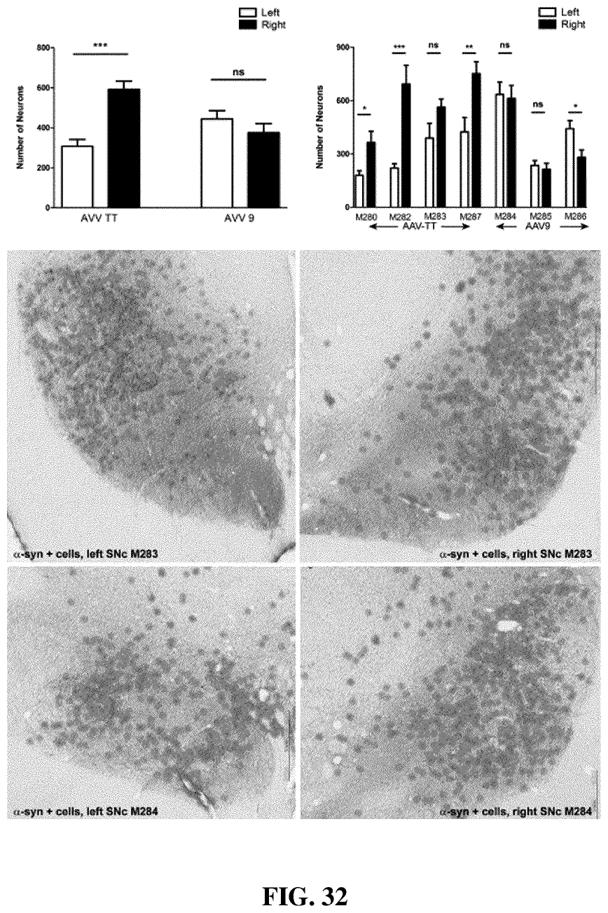

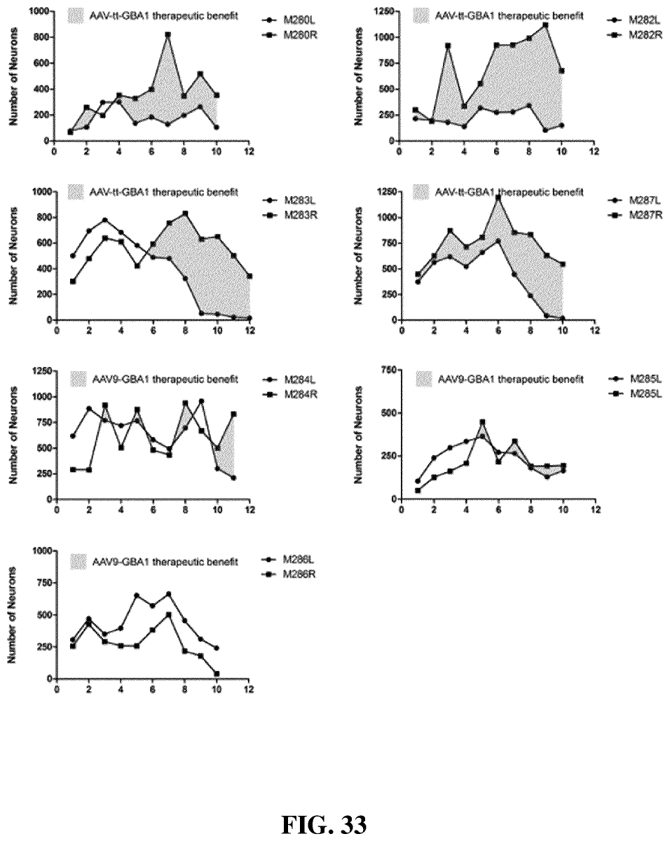

To assess to what extent the AAV-mediated enhancement of GCase activity was efficient for conducting a noticeable clearance of alpha-synuclein, 14 coronal sections stained for α-syn and comprising the whole rostrocaudal extent of the left and right substantia nigra were used. Analysis was conducted using Aiforia®. Obtained data are illustrated in FIGS. 32 and 33.

Left vs. right differences reflecting therapeutic benefit were consistently found to be much higher in animals injected with AAV-TT-GBA1 than in animals injected with AAV9-GBA1. Indeed, the therapeutic benefit of AAV-TT-GBA1 is maintained through most of the rostrocaudal extent of the substantia nigra. Indeed, it is also worth noting that the effect of AAV9-GBA1 in alpha-synuclein clearance was found to be minimal—if any—for all animals (animal M281 not considered for analysis due to surgical mistargeting with AAV9-SynA53T).

Ratios TH+/α-syn+

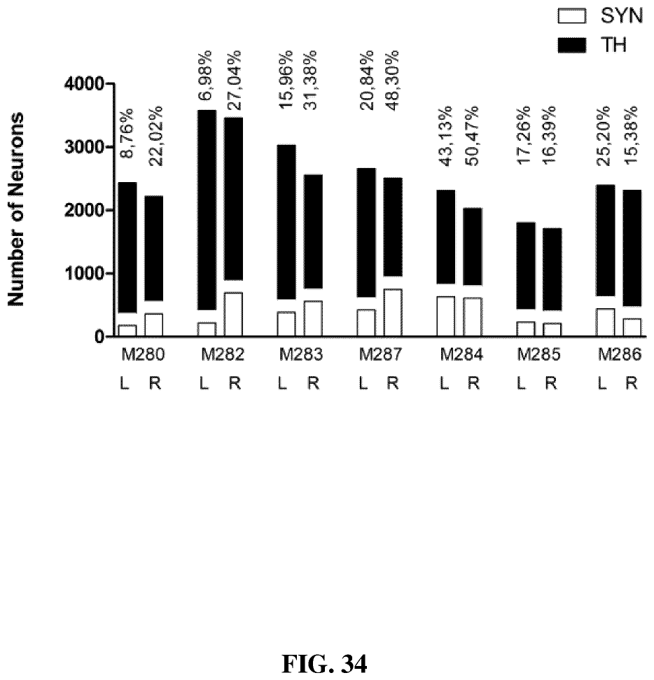

In an attempt to evaluate the overall extent of the induced synucleinopathy with AAV9-SynA53T, FIG. 34 illustrates the observed ratios between TH+ cells and -syn+ cells. From the obtained data it can be concluded that the induced synucleinopathy was of a moderate extent.

These data clearly support for a superior performance of AAV-TT-GBA1 vs. AAV9-GBA1 in terms of (1) better preservation of the nigrostriatal innervation, (2) better neuroprotective effect for dopaminergic neurons and (3) better efficacy for alpha-synuclein clearance.

7. Sequences for Use in Practicing the Invention

Sequences for use in practicing the invention are described below (non-limiting list):

SEQ ID NO: 1:
Human GBA1 coding nucleotide sequence:
ATGGCTGGCAGTCTTACAGGTCTCCTGCTCCTGCAAGCTGTCTCTTGGGCTTCTGGGGCCAGGCCCTG
TATCCCCAAATCCTTTGGATACTCATCTGTGGTGTGTGTTTGTAATGCCACTTATTGTGATAGCTTTG
ACCCCCCCACCTTTCCTGCACTGGGCACCTTTTCAAGGTATGAATCTACCAGGTCTGGGAGGAGGATG
GAGCTGAGTATGGGGCCCATCCAAGCAAACCATACTGGCACTGGCTTGCTGCTGACACTGCAACCTGA
ACAGAAGTTCCAGAAAGTGAAGGGCTTTGGAGGAGCCATGACTGATGCTGCTGCCCTCAATATTTTGG
CCCTGAGCCCCCCTGCTCAGAATCTCCTTTTGAAATCATACTTCTCTGAGGAGGGAATTGGATACAAT
ATCATCAGGGTGCCAATGGCCTCATGTGACTTTAGTATTAGGACTTACACCTATGCTGATACCCCTGA
TGATTTCCAGCTGCATAACTTCTCATTGCCTGAGGAGGATACCAAATTGAAGATCCCACTCATTCACA
GGGCCCTGCAACTGGCTCAGAGACCAGTGTCATTGCTGGCCTCCCCCTGGACCTCCCCAACTTGGCTC
AAAACCAATGGGGCTGTCAATGGTAAGGGCTCTCTTAAGGGGCAGCCTGGAGACATTTACCATCAGAC
CTGGGCCAGGTATTTTGTGAAGTTCCTGGATGCTTATGCTGAGCACAAATTGCAATTTTGGGCTGTTA
CAGCTGAGAATGAACCCTCTGCAGGACTGCTGTCTGGCTATCCTTTCCAGTGCCTGGGCTTTACCCCT
GAGCATCAGAGGGATTTCATTGCCAGGGACCTGGGACCTACTCTTGCCAATAGCACACACCATAATGT
GAGGCTTCTGATGCTTGATGACCAGAGACTTCTGCTGCCACACTGGGCCAAGGTTGTCCTGACAGATC
CTGAGGCTGCCAAGTATGTTCATGGGATTGCTGTGCACTGGTATCTGGACTTCCTTGCTCCAGCTAAG
GCCACCCTGGGAGAAACACACAGGTTGTTTCCCAATACAATGCTTTTTGCATCAGAGGCCTGTGTGGG
CAGTAAATTTTGGGAGCAGTCTGTTAGGCTGGGGAGCTGGGATAGAGGAATGCAATACTCCCATTCTA
TCATCACCAATCTGCTCTACCATGTGGTGGGGTGGACTGACTGGAACCTTGCCCTTAACCCTGAGGGT
GGCCCCAATTGGGTCAGGAATTTTGTGGATAGTCCCATCATTGTGGATATCACCAAGGACACATTCTA
TAAGCAACCAATGTTCTATCACCTGGGTCACTTTAGTAAGTTTATCCCTGAGGGGTCCCAGAGGGTGG
GACTGGTGGCTTCCCAGAAGAATGATCTGGATGCTGTGGCCCTGATGCACCCTGATGGCAGTGCTGTG
GTTGTTGTTCTCAATAGAAGCTCTAAAGATGTGCCCTTGACCATCAAAGATCCAGCTGTGGGATTTCT
GGAAACAATTTCCCCTGGTTATAGCATCCACACTTACCTTTGGAGAAGGCAGTGA
SEQ ID NO: 2:
nGUSB promoter nucleotide sequence
ATTCCTGCTGGGAAAAGCAAGTGGAGGTGCTCCTTGAAGAAACAGGGGGATCCCACCGATCTCAGGGG
TTCTGTTCTGGCCTGCGGCCCTGGATCGTCCAGCCTGGGTCGGGGTGGGGAGCAGACCTCGCCCTTAT
CGGCTGGGGCTGAGGGTGAGGGTCCCGTTTCCCCAAAGGCCTAGCCTGGGGTTCCAGCCACAAGCCCT
ACCGGGCAGCGCCCGGCCCCGCCCCTCCAGGCCTGGCACTCGTCCTCAACCAAGATGGCGCGGATGGC
TTCAGGCGCATCACGACACCGGCGCGTCACGCGACCCGCCCTACGGGCACCTCCCGCGCTTTTCTTAG
CGCCGCAGACGGTGGCCGAGCGGGGGACCGGGAAGCATGGCCCGGGCT
SEQ ID NO: 3:
Bovine growth hormone (BGH) polyadenylation signal
GCCCTGTGCCTTCTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAGCAGGCATG
CACT
SEQ ID NO: 4:
pAAV.nGUSB.GBA1 nucleotide sequence
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATATGCGGTGTGAAATACCGC
ACAGATGCGTAAGGAGAAAATACCGCATCAGGCGCCATTCGCCATTCAGGCTGCGCAACTGTTGGGAA
GGGCGATCGGTGCGGGCCTCTTCGCTATTACGCCAGCTGGCGAAAGGGGGATGTGCTGCAAGGCGATT
AAGTTGGGTAACGCCAGGGTTTTCCCAGTCACGACGTTGTAAAACGACGGCCAGTGAATTCGAGCTCG
GTACCTCGCGAATGCATCTAGAGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGAGAGGG
AGTGGCCAACTCCATCACTAGGGGTTCCTGGAGGGGTGGAGTCGTGACAGATCTGAATTCCTGCTGGG
AAAAGCAAGTGGAGGTGCTCCTTGAAGAAACAGGGGGATCCCACCGATCTCAGGGGTTCTGTTCTGGC
CTGCGGCCCTGGATCGTCCAGCCTGGGTCGGGGTGGGGAGCAGACCTCGCCCTTATCGGCTGGGGCTG
AGGGTGAGGGTCCCGTTTCCCCAAAGGCCTAGCCTGGGGTTCCAGCCACAAGCCCTACCGGGCAGCGC
CCGGCCCCGCCCCTCCAGGCCTGGCACTCGTCCTCAACCAAGATGGCGCGGATGGCTTCAGGCGCATC
ACGACACCGGCGCGTCACGCGACCCGCCCTACGGGCACCTCCCGCGCTTTTCTTAGCGCCGCAGACGG
TGGCCGAGCGGGGGACCGGGAAGCATGGCCCGGGCTGCAGCTCTAAGGTAAATATAAAATTTTTAAGT
GTATAATGTGTTAAACTACTGATTCTAATTGTTTCTCTCTTTTAGATTCCAACCTTTGGAACTCAATT
CAGCCACCATGGCTGGCAGTCTTACAGGTCTCCTGCTCCTGCAAGCTGTCTCTTGGGCTTCTGGGGCC
AGGCCCTGTATCCCCAAATCCTTTGGATACTCATCTGTGGTGTGTGTTTGTAATGCCACTTATTGTGA
TAGCTTTGACCCCCCCACCTTTCCTGCACTGGGCACCTTTTCAAGGTATGAATCTACCAGGTCTGGGA
GGAGGATGGAGCTGAGTATGGGGCCCATCCAAGCAAACCATACTGGCACTGGCTTGCTGCTGACACTG
CAACCTGAACAGAAGTTCCAGAAAGTGAAGGGCTTTGGAGGAGCCATGACTGATGCTGCTGCCCTCAA
TATTTTGGCCCTGAGCCCCCCTGCTCAGAATCTCCTTTTGAAATCATACTTCTCTGAGGAGGGAATTG
GATACAATATCATCAGGGTGCCAATGGCCTCATGTGACTTTAGTATTAGGACTTACACCTATGCTGAT
ACCCCTGATGATTTCCAGCTGCATAACTTCTCATTGCCTGAGGAGGATACCAAATTGAAGATCCCACT
CATTCACAGGGCCCTGCAACTGGCTCAGAGACCAGTGTCATTGCTGGCCTCCCCCTGGACCTCCCCAA
CTTGGCTCAAAACCAATGGGGCTGTCAATGGTAAGGGCTCTCTTAAGGGGCAGCCTGGAGACATTTAC
CATCAGACCTGGGCCAGGTATTTTGTGAAGTTCCTGGATGCTTATGCTGAGCACAAATTGCAATTTTG
GGCTGTTACAGCTGAGAATGAACCCTCTGCAGGACTGCTGTCTGGCTATCCTTTCCAGTGCCTGGGCT
TTACCCCTGAGCATCAGAGGGATTTCATTGCCAGGGACCTGGGACCTACTCTTGCCAATAGCACACAC
CATAATGTGAGGCTTCTGATGCTTGATGACCAGAGACTTCTGCTGCCACACTGGGCCAAGGTTGTCCT
GACAGATCCTGAGGCTGCCAAGTATGTTCATGGGATTGCTGTGCACTGGTATCTGGACTTCCTTGCTC
CAGCTAAGGCCACCCTGGGAGAAACACACAGGTTGTTTCCCAATACAATGCTTTTTGCATCAGAGGCC
TGTGTGGGCAGTAAATTTTGGGAGCAGTCTGTTAGGCTGGGGAGCTGGGATAGAGGAATGCAATACTC
CCATTCTATCATCACCAATCTGCTCTACCATGTGGTGGGGTGGACTGACTGGAACCTTGCCCTTAACC
CTGAGGGTGGCCCCAATTGGGTCAGGAATTTTGTGGATAGTCCCATCATTGTGGATATCACCAAGGAC
ACATTCTATAAGCAACCAATGTTCTATCACCTGGGTCACTTTAGTAAGTTTATCCCTGAGGGGTCCCA
GAGGGTGGGACTGGTGGCTTCCCAGAAGAATGATCTGGATGCTGTGGCCCTGATGCACCCTGATGGCA
GTGCTGTGGTTGTTGTTCTCAATAGAAGCTCTAAAGATGTGCCCTTGACCATCAAAGATCCAGCTGTG
GGATTTCTGGAAACAATTTCCCCTGGTTATAGCATCCACACTTACCTTTGGAGAAGGCAGTGAAAATG
AAGGCCTGATAATTGCACCACCAGGCCTGATAGGCCCTGTGCCTTCTAGTTGCCAGCCATCTGTTGTT
TGCCCCTCCCCCGTGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTGGGGCAGGACAGCA
AGGGGGAGGATTGGGAAGACAATAGCAGGCATGCACTAGTCCACTCCCTCTCTGCGCGCTCGCTCGCT
CACTGAGGCCGGGCGACCAAAGGTCGCCCGACGCCCGGGCTTTGCCCGGGCGGCCTCAGTGAGCGAGC
GAGCGCGCAGAGAGGGATCTAGATATCGGATCCCGGGCCCGTCGACTGCAGAGGCCTGCATGCAAGCT
TGGCGTAATCATGGTCATAGCTGTTTCCTGTGTGAAATTGTTATCCGCTCACAATTCCACACAACATA
CGAGCCGGAAGCATAAAGTGTAAAGCCTGGGGTGCCTAATGAGTGAGCTAACTCACATTAATTGCGTT
GCGCTCACTGCCCGCTTTCCAGTCGGGAAACCTGTCGTGCCAGCTGCATTAATGAATCGGCCAACGCG
CGGGGAGAGGCGGTTTGCGTATTGGGCGCTCTTCCGCTTCCTCGCTCACTGACTCGCTGCGCTCGGTC
GTTCGGCTGCGGCGAGCGGTATCAGCTCACTCAAAGGCGGTAATACGGTTATCCACAGAATCAGGGGA
TAACGCAGGAAAGAACATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGCTCAAGTCAGAGGTGG
CGAAACCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCTACAG
AGTTCTTGAAGTGGTGGCCTAACTACGGCTACACTAGAAGAACAGTATTTGGTATCTGCGCTCTGCTG
AAGCCAGTTACCTTCGGAAAAAGAGTTGGTAGCTCTTGATCCGGCAAACAAACCACCGCTGGTAGCGG
TGGTTTTTTTGTTTGCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGTCATGAGATTATCA
AAAAGGATCTTCACCTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGC
CCCAGTGCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACGATCAAGGCGAGT
TACATGATCCCCCATGTTGTGCAAAAAAGCGGTTAGCTCCTTCGGTCCTCCGATCGTTGTCAGAAGTA
AGTTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTTGAATGT
ATTTAGAAAAATAAACAAATAGGGGTTCCGCGCACATTTCCCCGAAAAGTGCCACCTGACGTCTAAGA
AACCATTATTATCATGACATTAACCTATAAAAATAGGCGTATCACGAGGCCCTTTCGTC
SEQ ID NO: 5
Amino acid sequence of human glucocerebrosidase without signal peptide
sequence (as encoded by SEQ ID NO: 1)
ARPCIPKSFGYSSVVCVCNATYCDSFDPPTFPALGTFSRYESTRSGRRMELSMGPIQANHTGTGLLLT
LQPEQKFQKVKGFGGAMTDAAALNILALSPPAQNLLLKSYFSEEGIGYNIIRVPMASCDFSIRTYTYA
DTPDDFQLHNFSLPEEDTKLKIPLIHRALQLAQRPVSLLASPWTSPTWLKTNGAVNGKGSLKGQPGDI
YHQTWARYFVKFLDAYAEHKLQFWAVTAENEPSAGLLSGYPFQCLGFTPEHQRDFIARDLGPTLANST
HHNVRLLMLDDQRLLLPHWAKVVLTDPEAAKYVHGIAVHWYLDFLAPAKATLGETHRLFPNTMLFASE
ACVGSKFWEQSVRLGSWDRGMQYSHSIITNLLYHVVGWTDWNLALNPEGGPNWVRNFVDSPIIVDITK
DTFYKQPMFYHLGHFSKFIPEGSQRVGLVASQKNDLDAVALMHPDGSAVVVVLNRSSKDVPLTIKDPA
VGFLETISPGYSIHTYLWRRQ
SEQ ID NO: 6
Full amino acid sequence of human glucocerebrosidase including short
signal peptide (as encoded by SEQ ID NO: 1)
MAGSLTGLLLLQAVSWASGARPCIPKSFGYSSVVCVCNATYCDSFDPPTFPALGTFSRYESTRSGRRM
ELSMGPIQANHTGTGLLLTLQPEQKFQKVKGFGGAMTDAAALNILALSPPAQNLLLKSYFSEEGIGYN
IIRVPMASCDFSIRTYTYADTPDDFQLHNFSLPEEDTKLKIPLIHRALQLAQRPVSLLASPWTSPTWL
KTNGAVNGKGSLKGQPGDIYHQTWARYFVKFLDAYAEHKLQFWAVTAENEPSAGLLSGYPFQCLGFTP
EHQRDFIARDLGPTLANSTHHNVRLLMLDDQRLLLPHWAKVVLTDPEAAKYVHGIAVHWYLDFLAPAK
ATLGETHRLFPNTMLFASEACVGSKFWEQSVRLGSWDRGMQYSHSIITNLLYHVVGWTDWNLALNPEG
GPNWVRNFVDSPIIVDITKDTFYKQPMFYHLGHFSKFIPEGSQRVGLVASQKNDLDAVALMHPDGSAV
VVVLNRSSKDVPLTIKDPAVGFLETISPGYSIHTYLWRRQ
SEQ ID NO: 7
Full nucleotide sequence of coding sequence of human GBA1 gene (wild
type)
Human glucocerebrosidase mRNA, complete cds, GenBank: M19285.1
>M19285.1: 123-1733 Human glucocerebrosidase mRNA, complete cds
ATGGAGTTTTCAAGTCCTTCCAGAGAGGAATGTCCCAAGCCTTTGAGTAGGGTAAGCATCATGGCTGG
CAGCCTCACAGGTTTGCTTCTACTTCAGGCAGTGTCGTGGGCATCAGGTGCCCGCCCCTGCATCCCTA
AAAGCTTCGGCTACAGCTCGGTGGTGTGTGTCTGCAATGCCACATACTGTGACTCCTTTGACCCCCCG
ACCTTTCCTGCCCTTGGTACCTTCAGCCGCTATGAGAGTACACGCAGTGGGCGACGGATGGAGCTGAG
TATGGGGCCCATCCAGGCTAATCACACGGGCACAGGCCTGCTACTGACCCTGCAGCCAGAACAGAAGT
TCCAGAAAGTGAAGGGATTTGGAGGGGCCATGACAGATGCTGCTGCTCTCAACATCCTTGCCCTGTCA
CCCCCTGCCCAAAATTTGCTACTTAAATCGTACTTCTCTGAAGAAGGAATCGGATATAACATCATCCG
GGTACCCATGGCCAGCTGTGACTTCTCCATCCGCACCTACACCTATGCAGACACCCCTGATGATTTCC
AGTTGCACAACTTCAGCCTCCCAGAGGAAGATACCAAGCTCAAGATACCCCTGATTCACCGAGCCCTG
CAGTTGGCCCAGCGTCCCGTTTCACTCCTTGCCAGCCCCTGGACATCACCCACTTGGCTCAAGACCAA
TGGAGCGGTGAATGGGAAGGGGTCACTCAAGGGACAGCCCGGAGACATCTACCACCAGACCTGGGCCA
GATACTTTGTGAAGTTCCTGGATGCCTATGCTGAGCACAAGTTACAGTTCTGGGCAGTGACAGCTGAA
AATGAGCCTTCTGCTGGGCTGTTGAGTGGATACCCCTTCCAGTGCCTGGGCTTCACCCCTGAACATCA
GCGAGACTTCATTGCCCGTGACCTAGGTCCTACCCTCGCCAACAGTACTCACCACAATGTCCGCCTAC
TCATGCTGGATGACCAACGCTTGCTGCTGCCCCACTGGGCAAAGGTGGTACTGACAGACCCAGAAGCA
GCTAAATATGTTCATGGCATTGCTGTACATTGGTACCTGGACTTTCTGGCTCCAGCCAAAGCCACCCT
AGGGGAGACACACCGCCTGTTCCCCAACACCATGCTCTTTGCCTCAGAGGCCTGTGTGGGCTCCAAGT
TCTGGGAGCAGAGTGTGCGGCTAGGCTCCTGGGATCGAGGGATGCAGTACAGCCACAGCATCATCACG
AACCTCCTGTACCATGTGGTCGGCTGGACCGACTGGAACCTTGCCCTGAACCCCGAAGGAGGACCCAA
TTGGGTGCGTAACTTTGTCGACAGTCCCATCATTGTAGACATCACCAAGGACACGTTTTACAAACAGC
CCATGTTCTACCACCTTGGCCACTTCAGCAAGTTCATTCCTGAGGGCTCCCAGAGAGTGGGGCTGGTT
GCCAGTCAGAAGAACGACCTGGACGCAGTGGCACTGATGCATCCCGATGGCTCTGCTGTTGTGGTCGT
GCTAAACCGCTCCTCTAAGGATGTGCCTCTTACCATCAAGGATCCTGCTGTGGGCTTCCTGGAGACAA
TCTCACCTGGCTACTCCATTCACACCTACCTGTGGCATCGCCAGTGA
SEQ ID NO: 8
Full amino acid sequence of human glucocerebrosidase including long
signal peptide (NCBI reference: NP_001005742.1, lysosomal acid
glucosylceramidase isoform 1 precursor)
MEFSSPSREECPKPLSRVSIMAGSLTGLLLLQAVSWASGARPCIPKSFGYSSVVCVCNATYCDSFDPP
TFPALGTFSRYESTRSGRRMELSMGPIQANHTGTGLLLTLQPEQKFQKVKGFGGAMTDAAALNILALS
PPAQNLLLKSYFSEEGIGYNIIRVPMASCDFSIRTYTYADTPDDFQLHNFSLPEEDTKLKIPLIHRAL
QLAQRPVSLLASPWTSPTWLKTNGAVNGKGSLKGQPGDIYHQTWARYFVKFLDAYAEHKLQFWAVTAE
NEPSAGLLSGYPFQCLGFTPEHQRDFIARDLGPTLANSTHHNVRLLMLDDQRLLLPHWAKVVLTDPEA
AKYVHGIAVHWYLDFLAPAKATLGETHRLFPNTMLFASEACVGSKFWEQSVRLGSWDRGMQYSHSIIT
NLLYHVVGWTDWNLALNPEGGPNWVRNFVDSPIIVDITKDTFYKQPMFYHLGHFSKFIPEGSQRVGLV
ASQKNDLDAVALMHPDGSAVVVVLNRSSKDVPLTIKDPAVGFLETISPGYSIHTYLWRRQ
SEQ ID NO: 9
Nucleotide sequence of the CAG promoter
ctagttattaatagtaatcaattacggggtcattagttcatagcccatatatggagttccgcgttaca
taacttacggtaaatggcccgcctggctgaccgcccaacgacccccgcccattgacgtcaataatgac
gtatgttcccatagtaacgccaatagggactttccattgacgtcaatgggtggagtatttacggtaaa
ctgcccacttggcagtacatcaagtgtatcatatgccaagtacgccccctattgacgtcaatgacggt
aaatggcccgcctggcattatgcccagtacatgaccttatgggactttcctacttggcagtacatcta
cgtattagtcatcgctattaccatggtcgaggtgagccccacgttctgcttcactctccccatctccc
ccccctccccacccccaattttgtatttatttattttttaattattttgtgcagcgatgggggcgggg
ggggggggggggcgcgcgccaggcggggcggggcggggcgaggggcggggcggggcgaggcggagagg
tgcggcggcagccaatcagagcggcgcgctccgaaagtttccttttatggcgaggcggcggcggcggc
ggccctataaaaagcgaagcgcgcggcgggcg
SEQ ID NO: 10
GFP encoding nucleotide sequence
Atggtgagcaagggcgaggagctgttcaccggggtggtgcccatcctggtcgagctggacggcgacgt
aaacggccacaagttcagcgtgtccggcgagggcgagggcgatgccacctacggcaagctgaccctga
agttcatctgcaccaccggcaagctgcccgtgccctggcccaccctcgtgaccaccctgacctacggc
gtgcagtgcttcagccgctaccccgaccacatgaagcagcacgacttcttcaagtccgccatgcccga
aggctacgtccaggagcgcaccatcttcttcaaggacgacggcaactacaagacccgcgccgaggtga
agttcgagggcgacaccctggtgaaccgcatcgagctgaagggcatcgacttcaaggaggacggcaac
atcctggggcacaagctggagtacaactacaacagccacaacgtctatatcatggccgacaagcagaa
gaacggcatcaaggtgaacttcaagatccgccacaacatcgaggacggcagcgtgcagctcgccgacc
actaccagcagaacacccccatcggcgacggccccgtgctgctgcccgacaaccactacctgagcacc
cagtccgccctgagcaaagaccccaacgagaagcgcgatcacatggtcctgctggagttcgtgaccgc
cgccgggatcactctcggcatggacgagctgtacaagtga
SEQ ID NO: 11
Human GBA1 coding nucleotide sequence (IDT optimized sequence)
atggagttctcatctccctcacgagaagaatgtccgaaacctctttcaagagtaagcatcatggccgg
cagcttgaccggtcttttgttgttgcaggccgtgtcctgggcctcaggtgctaggccatgcattccta
aatccttcggctatagtagcgtggtttgcgtctgcaacgccacatactgtgacagtttcgatccacct
accttcccagcgctgggtaccttctcacggtatgaatcaacgcgatcagggcgcagaatggaactttc
aatggggccaatccaagctaaccacacgggaacgggtcttctgctgacgctccaaccggaacaaaagt
tccaaaaggtaaaaggctttggaggtgcgatgactgatgccgcagcactcaacatcctggcgctctca
ccgccggcacaaaatttgctgttgaagagttatttctcagaagaagggatcggttacaacatcatacg
ggtcccgatggcgagctgtgacttttctataagaacatatacctatgcggatacgcccgacgatttcc
aacttcataattttagtctgcctgaggaagacacaaagttgaagataccgctgatacacagagcattg
cagcttgctcaacgaccggtcagcttgcttgccagcccatggacaagtccaacatggcttaagaccaa
tggcgcggttaatggcaagggatccctgaagggccagccgggagacatctatcatcaaacttgggcgc
ggtattttgtcaagttcttggacgcctacgctgagcacaaactgcagttctgggccgttaccgccgaa
aatgaaccatccgccggactgctttctggctaccctttccaatgtcttggctttacgcctgaacacca
aagagacttcattgctcgggaccttggtccaacgctcgcgaacagtactcatcataatgtacgactct
tgatgctcgatgaccagcgactgttgcttccacattgggccaaggtagttctgaccgaccccgaagcc
gctaaatacgtccacggcattgctgtccattggtaccttgactttttggctcccgcaaaagccactct
gggtgaaacacacagactctttccaaacacgatgcttttcgcatcagaagcctgcgtcggaagtaaat
tttgggaacagtcagtaaggttgggtagttgggatcgcgggatgcaatatagtcatagcattattacc
aacttgctttatcacgtcgttgggtggacagattggaacctcgcgttgaatcctgaaggcggccctaa
ttgggtaagaaactttgttgattcacctattatcgtcgacataaccaaggacacattctacaagcaac
cgatgttctatcaccttgggcatttcagtaaattcataccagagggcagccagcgcgtcgggttggta
gcctctcaaaaaaacgatttggatgcggtcgctctgatgcatcccgacgggagcgcagtagtcgttgt
ccttaaccgaagctccaaggatgtacccctcacgattaaggaccctgctgtcgggttccttgaaacta
taagtcccggctatagtattcatacttatctctggagaagacagtga
SEQ ID NO: 12
Human GBA1 coding nucleotide sequence (GenScript optimized
sequence)
ATGGAGTTTTCAAGCCCCTCACGGGAAGAGTGCCCTAAGCCCCTGTCACGGGTCTCAATTATGGCCGG
GAGCCTGACTGGCCTGCTGCTGCTGCAGGCCGTGAGCTGGGCATCAGGAGCCAGGCCTTGCATCCCAA
AGTCTTTCGGCTACAGCTCCGTGGTGTGCGTGTGCAACGCCACCTATTGTGACTCCTTCGATCCCCCT
ACCTTTCCCGCCCTGGGCACATTTTCTAGATACGAGTCTACACGCAGCGGCCGGAGAATGGAGCTGAG
CATGGGCCCTATCCAGGCCAATCACACCGGAACAGGCCTGCTGCTGACCCTGCAGCCAGAGCAGAAGT
TCCAGAAGGTGAAGGGCTTTGGAGGAGCAATGACAGACGCAGCCGCCCTGAACATCCTGGCCCTGTCC
CCACCCGCCCAGAATCTGCTGCTGAAGTCCTACTTCTCTGAGGAGGGCATCGGCTATAACATCATCCG
GGTGCCCATGGCCAGCTGCGACTTTTCCATCAGAACCTACACATATGCCGATACCCCTGACGATTTCC
AGCTGCACAATTTTTCCCTGCCAGAGGAGGATACAAAGCTGAAGATCCCCCTGATCCACCGGGCCCTG
CAGCTGGCACAGCGGCCCGTGAGCCTGCTGGCCAGCCCCTGGACCTCCCCTACATGGCTGAAGACCAA
CGGCGCCGTGAATGGCAAGGGCTCTCTGAAGGGACAGCCAGGCGACATCTACCACCAGACATGGGCCA
GATATTTCGTGAAGTTTCTGGATGCCTACGCCGAGCACAAGCTGCAGTTCTGGGCCGTGACCGCAGAG
AACGAGCCTTCTGCCGGCCTGCTGAGCGGCTATCCCTTCCAGTGCCTGGGCTTTACACCTGAGCACCA
GCGGGACTTTATCGCCAGAGATCTGGGCCCAACCCTGGCCAACTCCACACACCACAATGTGAGGCTGC
TGATGCTGGACGATCAGCGCCTGCTGCTGCCTCACTGGGCCAAGGTGGTGCTGACCGACCCAGAGGCC
GCCAAGTACGTGCACGGCATCGCCGTGCACTGGTATCTGGATTTCCTGGCACCAGCAAAGGCCACCCT
GGGAGAGACACACCGGCTGTTCCCTAACACCATGCTGTTTGCCAGCGAGGCCTGCGTGGGCTCCAAGT
TTTGGGAGCAGTCCGTGAGGCTGGGATCTTGGGACAGGGGCATGCAGTACTCCCACTCTATCATCACC
AATCTGCTGTATCACGTGGTGGGCTGGACAGACTGGAACCTGGCCCTGAATCCAGAGGGCGGCCCCAA
CTGGGTGAGAAATTTCGTGGATAGCCCCATCATCGTGGACATCACCAAGGATACATTCTACAAGCAGC
CAATGTTTTATCACCTGGGCCACTTCTCTAAGTTTATCCCAGAGGGCAGCCAGAGGGTGGGCCTGGTG
GCCAGCCAGAAGAACGACCTGGATGCCGTGGCCCTGATGCACCCTGATGGCTCCGCCGTGGTGGTGGT
GCTGAATCGCTCTAGCAAGGACGTGCCTCTGACCATCAAGGATCCAGCCGTGGGCTTCCTGGAGACTA
TTTCCCCCGGCTATTCAATTCATACCTATCTGTGGAGAAGGCAGTGA
SEQ ID NO: 13
Nucleotide sequence of the hSyn promoter (NCBI ref. NG_008437.1)
AGTGCAAGTGGGTTTTAGGACCAGGATGAGGCGGGGTGGGGGTGCCTACCTGACGACCGACCCCGACC
CACTGGACAAGCACCCAACCCCCATTCCCCAAATTGCGCATCCCCTATCAGAGAGGGGGAGGGGAAAC
AGGATGCGGCGAGGCGCGTGCGCACTGCCAGCTTCAGCACCGCGGACAGTGCCTTCGCCCCCGCCTGG
CGGCGCGCGCCACCGCCGCCTCAGCACTGAAGGCGCGCTGACGTCACTCGCCGGTCCCCCGCAAACTC
CCCTTCCCGGCCACCTTGGTCGCGTCCGCGCCGCCGCCGGCCCAGCCGGACCGCACCACGCGAGGCGC
GAGATAGGGGGGCACGGGCGCGACCATCTGCGCTGCGGCGCCGGCGACTCAGCGCTGCCTCAGTCTGC
GGTGGGCAGCGGAGGAGTCGTGTCGTGCCTGAGAGCGCAG
SEQ ID NO: 14
Amino acid sequence of AAVTT capsid protein
MAADGYLPDWLEDTLSEGIRQWWKLKPGPPPPKPAERHKDDSRGLVLPGYKYLGPFNGLDKGEPVNEA
DAAALEHDKAYDRQLDSGDNPYLKYNHADAEFQERLKEDTSFGGNLGRAVFQAKKRILEPLGLVEEPV
KTAPGKKRPVEHSPAEPDSSSGTGKSGQQPARKRLNFGQTGDADSVPDPQPLGQPPAAPSGLGTNTMA
SGSGAPMADNNEGADGVGNSSGNWHCDSTWMGDRVITTSTRTWALPTYNNHLYKQISSQSGASNDNHY
FGYSTPWGYFDFNRFHCHFSPRDWQRLINNNWGFRPKRLSFKLFNIQVKEVTQNDGTTTIANNLTSTV
QVFTDSEYQLPYVLGSAHQGCLPPFPADVFMVPQYGYLTLNNGSQAVGRSSFYCLEYFPSQMLRTGNN
FTFSYTFEDVPFHSSYAHSQSLDRLMNPLIDQYLYYLSRTNTPSGTTTMSRLQFSQAGASDIRDQSRN
WLPGPCYRQQRVSKTAADNNNSDYSWTGATKYHLNGRDSLVNPGPAMASHKDDEEKYFPQSGVLIFGK
QDSGKTNVDIEKVMITDEEEIRTTNPVATEQYGSVSTNLQSGNTQAATSDVNTQGVLPGMVWQDRDVY
LQGPIWAKIPHTDGHFHPSPLMGGFGLKHPPPQILIKNTPVPANPSTTFSAAKFASFITQYSTGQVSV
EIEWELQKENSKRWNPEIQYTSNYNKSVNVDFTVDTNGVYSEPRPIGTRYLTRNL
SEQ ID NO: 15
Nucleotide sequence of Flip ITR of AAV2
TTGGCCACTCCCTCTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGAGAGGGAGTGGCCAACTCCATCACTA
GGGGTTCCT
SEQ ID NO: 16
Nucleotide sequence of Flop ITR of AAV2
AGGAACCCCTAGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGAGAGGG
AGTGGCCAA
SEQ ID NO: 17:
Human GBA1 isoform 2 amino acid sequence (NCBI Ref. NP_001165282.1)
MELSMGPIQANHTGTGLLLTLQPEQKFQKVKGFGGAMTDAAALNILALSPPAQNLLLKSYFSEEGIGY
NIIRVPMASCDFSIRTYTYADTPDDFQLHNFSLPEEDTKLKIPLIHRALQLAQRPVSLLASPWTSPTW
LKTNGAVNGKGSLKGQPGDIYHQTWARYFVKFLDAYAEHKLQFWAVTAENEPSAGLLSGYPFQCLGFT
PEHQRDFIARDLGPTLANSTHHNVRLLMLDDQRLLLPHWAKVVLTDPEAAKYVHGIAVHWYLDFLAPA
KATLGETHRLFPNTMLFASEACVGSKFWEQSVRLGSWDRGMQYSHSIITNLLYHVVGWTDWNLALNPE
GGPNWVRNFVDSPIIVDITKDTFYKQPMFYHLGHFSKFIPEGSQRVGLVASQKNDLDAVALMHPDGSA
VVVVLNRSSKDVPLTIKDPAVGFLETISPGYSIHTYLWRRQ
SEQ ID NO: 18:
Human GBA1 isoform 3 amino acid sequence (NCBI ref. NP_001165283.1)
MEFSSPSREECPKPLSRVSIMAGSLTGLLLLQAVSWASGARPCIPKSFGYSSVVCVCNATYCDSFDPP
TFPALGTFSRYESTRSGRRMELSMGPIQANHTGTGIGYNIIRVPMASCDFSIRTYTYADTPDDFQLHN
FSLPEEDTKLKIPLIHRALQLAQRPVSLLASPWTSPTWLKTNGAVNGKGSLKGQPGDIYHQTWARYFV
KFLDAYAEHKLQFWAVTAENEPSAGLLSGYPFQCLGFTPEHQRDFIARDLGPTLANSTHHNVRLLMLD
DQRLLLPHWAKVVLTDPEAAKYVHGIAVHWYLDFLAPAKATLGETHRLFPNTMLFASEACVGSKFWEQS
VRLGSWDRGMQYSHSIITNLLYHVVGWTDWNLALNPEGGPNWVRNFVDSPIIVDITKDTFYKQPMFYH
LGHFSKFIPEGSQRVGLVASQKNDLDAVALMHPDGSAVVVVLNRSSKDVPLTIKDPAVGFLETISPGY
SIHTYLWRRQ
SEQ ID NO: 19:
Full nucleotide sequence of coding sequence of human GBA1 gene
atggagttttcaagtccttccagagaggaatgtcccaagcctttgagtagggtaagcatcatggctgg
cagcctcacaggattgcttctacttcaggcagtgtcgtgggcatcaggtgcccgcccctgcatcccta
aaagcttcggctacagctcggtggtgtgtgtctgcaatgccacatactgtgactcctttgaccccccg
acctttcctgcccttggtaccttcagccgctatgagagtacacgcagtgggcgacggatggagctgag
tatggggcccatccaggctaatcacacgggcacaggcctgctactgaccctgcagccagaacagaagt
tccagaaagtgaagggatttggaggggccatgacagatgctgctgctctcaacatccttgccctgtca
ccccctgcccaaaatttgctacttaaatcgtacttctctgaagaaggaatcggatataacatcatccg
ggtacccatggccagctgtgacttctccatccgcacctacacctatgcagacacccctgatgatttcc
agttgcacaacttcagcctcccagaggaagataccaagctcaagatacccctgattcaccgagccctg
cagttggcccagcgtcccgtttcactccttgccagcccctggacatcacccacttggctcaagaccaa
tggagcggtgaatgggaaggggtcactcaagggacagcccggagacatctaccaccagacctgggcca
gatactttgtgaagttcctggatgcctatgctgagcacaagttacagttctgggcagtgacagctgaa
aatgagccttctgctgggctgttgagtggataccccttccagtgcctgggcttcacccctgaacatca
gcgagacttcattgcccgtgacctaggtcctaccctcgccaacagtactcaccacaatgtccgcctac
tcatgctggatgaccaacgcttgctgctgccccactgggcaaaggtggtactgacagacccagaagca
gctaaatatgttcatggcattgctgtacattggtacctggactttctggctccagccaaagccaccct
aggggagacacaccgcctgttccccaacaccatgctctttgcctcagaggcctgtgtgggctccaagt
tctgggagcagagtgtgcggctaggctcctgggatcgagggatgcagtacagccacagcatcatcacg
aacctcctgtaccatgtggtcggctggaccgactggaaccttgccctgaaccccgaaggaggacccaa
ttgggtgcgtaactttgtcgacagtcccatcattgtagacatcaccaaggacacgttttacaaacagc
ccatgttctaccaccttggccacttcagcaagttcattcctgagggctcccagagagtggggctggtt
gccagtcagaagaacgacctggacgcagtggcactgatgcatcccgatggctctgctgttgtggtcgt
gctaaaccgctcctctaaggatgtgcctcttaccatcaaggatcctgctgtgggcttcctggagacaa
tctcacctggctactccattcacacctacctgtggcgtcgccagtga
SEQ ID NO: 20:
nGUSB promoter nucleotide sequence #2
ATTCCTGCTGGGAAAAGCAAGTGGAGGTGCTCCTTGAAGAAACAGGGGGATCCCACCGATCTCAGGGG
TTCTGTTCTGGCCTGCGGCCCTGGATCGTCCAGCCTGGGTCGGGGTGGGGAGCAGACCTCGCCCTTAT
CGGCTGGGGCTGAGGGTGAGGGTCCCGTTTCCCCAAAGGCCTAGCCTGGGGTTCCAGCCACAAGCCCT
ACCGGGCAGCGCCCGGCCCCGCCCCTCCAGGCCTGGCACTCGTCCTCAACCAAGATGGCGCGGATGGC
TTCAGGCGCATCACGACACCGGCGCGTCACGCGACCCGCCCTACGGGCACCTCCCGCGCTTTTCTTAG
CGCCGCAGACGGTGGCCGAGCGGGGGACCGGGAAGC
SEQ ID NO: 21
Nucleotide sequence of the CAG promoter # 2
ctagttattaatagtaatcaattacggggtcattagttcatagcccatatatggagttccgcgttaca
taacttacggtaaatggcccgcctggctgaccgcccaacgacccccgcccattgacgtcaataatgac
gtatgttcccatagtaacgccaatagggactttccattgacgtcaatgggtggagtatttacggtaaa
ctgcccacttggcagtacatcaagtgtatcatatgccaagtacgccccctattgacgtcaatgacggt
aaatggcccgcctggcattatgcccagtacatgaccttatgggactttcctacttggcagtacatcta
cgtattagtcatcgctattaccatggtcgaggtgagccccacgttctgcttcactctccccatctccc
ccccctccccacccccaattttgtatttatttattttttaattattttgtgcagcgatgggggcgggg
ggggggggggggcgcgcgccaggcggggcggggcggggcgaggggcggggcggggcgaggcggagagg
tgcggcggcagccaatcagagcggcgcgctccgaaagtttccttttatggcgaggcggcggcggcggc
ggccctataaaaagcgaagcgcgcggcgggcgggagtcgctgcgacgctgccttcgccccgtgccccg
ctccgccgccgcctcgcgccgcccgccccggctctgactgaccgcgttactcccacaggtgagcgggc
gggacggcccttctcctccgggctgtaattagcgcttggtttaatgacggcttgtttcttttctgtgg
ctgcgtgaaagccttgaggggctccgggagggccctttgtgcgggggggagcggctcggggggtgcgt
gcgtgtgtgtgtgcgtggggagcgccgcgtgcggcccgcgctgcccggcggctgtgagcgctgcgggc
gcggcgcggggctttgtgcgctccgcagtgtgcgcgaggggagcgcggccgggggcggtgccccgcgg
tgcggggggggctgcgaggggaacaaaggctgcgtgcggggtgtgtgcgtgggggggtgagcaggggg
tgtgggcgcggcggtcgggctgtaacccccccctgcacccccctccccgagttgctgagcacggcccg
gcttcgggtgcggggctccgtacggggcgtggcgcggggctcgccgtgccgggcggggggtggcggca
ggtgggggtgccgggcggggcggggccgcctcgggccggggagggctcgggggaggggcgcggcggcc
cccggagcgccggcggctgtcgaggcgcggcgagccgcagccattgccttttatggtaatcgtgcgag
agggcgcagggacttcctttgtcccaaatctgtgcggagccgaaatctgggaggcgccgccgcacccc
ctctagcgggcgcggggcgaagcggtgcggcgccggcaggaaggaaatgggcggggagggccttcgtg
cgtcgccgcgccgccgtccccttctccctctccagcctcggggctgtccgcggggggacggctgcctt
cgggggggacggggcagggcggggttcggcttctggcgtgtgaccggcggctctagagcctctgctaa
ccatgttcatgccttcttctttttcctacag
SEQ ID NO: 22
Nucleotide sequence of the Human Ubiquitin C (UbC) promoter short
version
Ggcctccgcgccgggttttggcgcctcccgcgggcgcccccctcctcacggcgagcgctgccacgtca
gacgaagggcgcagcgagcgtcctgatccttccgcccggacgctcaggacagcggcccgctgctcata
agactcggccttagaaccccagtatcagcagaaggacattttaggacgggacttgggtgactctaggg
cactggttttctttccagagagcggaacaggcgaggaaaagtagtcccttctcggcgattctgcggag
ggatctccgtggggcggtgaacgccgatgattatataaggacgcgccgggtgtggcacagctagttcc
gtcgcagccgggatttgggtcgcggttcttgtttgtggatcgctgtgatcgtcacttggt
SEQ ID NO: 23
Nucleotide sequence of the Human Ubiquitin C (UbC) promoter long
version
ccggaggcgcggcccaaaaccgcggagggcgcccgcggggggaggagtgccgctcgcgacggtgcagt
ctgcttcccgcgtcgctcgcaggactaggaaggcgggcctgcgagtcctgtcgccgggcgacgagtat
tctgagccggaatcttggggtcatagtcgtcttcctgtaaaatcctgccctgaacccactgagatccc
gtgaccaaaagaaaggtctctcgccttgtccgctccttttcatcagggaagagccgctaagacgcctc
cctagaggcaccccgccacttgcggctactaatatattcctgcgcggcccacaccgtgtcgatcaagg
cagcgtcggccctaaacccagcgccaagaacaaacacctagcgacactagcagtgaaccactcatcgc
ccgacgacccgaccggccccgaaagcaccggcggcccggcgagccaccctgccttcgcacacctctct
ggcggttcccgacatcagacccaggcgctcgttccaacgggacttgacccccaacccccctcgcgtcg
ttttaccgccgacaagggctcagaacttaccttctgcgaacactccgcccgacactccagcaactttg
ttccaccccccgtaccacccgccgttcttgggttccagaactccggaagcgattacgccctttcgaga
ataagcccactctacccgaccccgtggtagacccctgggactgcacttcaaacagtgactgacctctt
gagccaaacagcagacaacgcccccgccgtcaataccgccacggcaacccgtcacgtgggcatggaaa
ccctcgcgcgcgggagcagcacagcactgcagtgggcaagacaaccgaatattacgtcccaccccggt
ggacggccatccacacgccatccgaaaagaggcagcgtcctgcgtcccaagcccggatcccatccgag
aggacttagctgtccgcggcctggagaccactcccctccctattcactccgcagtcaaagaaaccagc
caaaatacatggatagaagaattcatcgacttcgaggccaaaacttgatacgcgagccccaaccgctc
acacaaaacacttcaaaaaatccgtggaaaactttacattagtaaacccagttatacattaaaagtca
caatctgatcatttaacaggcgatttaagaccggcaaaaaccgaaaaaacaatctg
SEQ ID NO: 24
Nucleotide sequence of the Phosphoglycerate kinase 1 (PGK) promoter
Gacccctctctccagccactaagccagttgctccctcggctgacggctgcacgcgaggcctccgaacg
tcttacgccttgtggcgcgcccgtccttgtcccgggtgtgatggcggggtgtggggcggagggcgtgg
cggggaagggccggcgacgagagccgcgcgggacgactcgtcggcgataaccggtgtcgggtagcgcc
agccgcgcgacggtaacgagggaccgcgacaggcagacgctcccatgatcactctgcacgccgaaggc
aaacagtgcaggccgtgcggcgcttggcgttccttggaagggctgaatccccgcctcgtccttcgcag
cggccccccgggtgttcccatcgccgcttctaggcccactgcgacgcttgcctgcacttcttacacgc
tctgggtcccagccgcggcgacgcaaagggccttggtgcgggtctcgtcggcgcagggacgcgtttgg
gtcccgacggaaccttttccgcgttggggttgggg
SEQ ID NO: 25
Nucleotide sequence of the CBA/CBh promoter #1
AGATGTACTGCCAAGTAGGAAAGTCCCGTAAGGTCATGTACTGGGCATAATGCCAGGCGGGCCATTTA
CCGTCATTGACGTCAATAGGGGGCGTACTTGGCATATGATACACTTGATGTACTGCCAAGTGGGCAGT
TTACCGTAAATACTCCACCCATTGACGTCAATGGAAAGTCCCTATTGGCGTTACTATGGGAACATACG
TCATTATTGACGTCAATGGGCGGGGGTCGTTGGGCGGTCAGCCAGGCGGGCCATTTACCGTAAGTTAT
GTAACGCGGAACTCCATATATGGGCTATGAACTAATGACCCCGTAATTGATTACTATTAACCACGTTC
TGCTTCACTCTCCCCATCTCCCCCCCCTCCCCACCCCCAATTTTGTATTTATTTATTTTTTAATTATT
TTGTGCAGCGATGGGGGCGGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGAACTGAAAAACCAGAAAGT
TAACTGGTAAGTTTAGTCTTTTTGTCTTTTATTTCAGGTCCTGGTGGTGCAAATCAAAGAACTGCTCC
TCAGTGGATGTTGCCTTTACTTCTAGGCCTGTACGGAAGTGTTACTTCTGCTCTAAAAGCT
SEQ ID NO: 26
Nucleotide sequence of the CBA/CBh promoter #2
AGATGTACTGCCAAGTAGGAAAGTCCCGTAAGGTCATGTACTGGGCATAATGCCAGGCGGGCCATTTA
CCGTCATTGACGTCAATAGGGGGCGTACTTGGCATATGATACACTTGATGTACTGCCAAGTGGGCAGT
TTACCGTAAATACTCCACCCATTGACGTCAATGGAAAGTCCCTATTGGCGTTACTATGGGAACATACG
TCATTATTGACGTCAATGGGCGGGGGTCGTTGGGCGGTCAGCCAGGCGGGCCATTTACCGTAAGTTAT
GTAACGCGGAACTCCATATATGGGCTATGAACTAATGACCCCGTAATTGATTACTATTAACCACGTTC
TGCTTCACTCTCCCCATCTCCCCCCCCTCCCCACCCCCAATTTTGTATTTATTTATTTTTTAATTATT
TTGTGCAGCGATGGGGGCGGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGGGAGTCGCTGCGTTGCCTT
CGCCCCGTGCCCCGCTCCGCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCAAGAGGTAAGGGTTTAAGGGAT
GGTTGGTTGGTGGGGTATTAATGTTTAATTACCTGTTTTACAGGCCTGAAATCACTTGGTTTTAGGTT
GG
SEQ ID NO: 27
Nucleotide sequence of the JeT promoter
GAATTCGGGCGGAGTTAGGGCGGAGCCAATCAGCGTGCGCCGTTCCGAAAGTTGCCTTTTATGGCTGG
GCGGAGAATGGGCGGTGAACGCCGATGATTATATAAGGACGCGCCGGGTGTGGCACAGCTAGTTCCGT
CGCAGCCGGGATTTGGGTCGCGGTTCTTGTTTGTGGATCCCTGTGATCGTCACTTGACA
SEQ ID NO: 28
Nucleotide sequence of the Human Growth Hormone polyA
ACGGGTGGCATCCCTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTTCTATAATATTAT
GGGGTGGAGGGGGGTGGTATGGAGCAAGGGGCAAGTTGGGAAGACAACCTGTAGGGCCTGCGGGGTCT
ATTGGGAACCAAGCTGGAGTGCAGTGGCACAATCTTGGCTCACTGCAATCTCCGCCTCCTGGGTTCAA
GCGATTCTCCTGCCTCAGCCTCCCGAGTTGTTGGGATTCCAGGCATGCATGACCAGGCTCAGCTAATT
TTTGTTTTTTTGGTAGAGACGGGGTTTCACCATATTGGCCAGGCTGGTCTCCAACTCCTAATCTCAGG
TGATCTACCCACCTTGGCCTCCCAAATTGCTGGGATTACAGGCGTGAACCACTGCTCCCTTCCCTGTC
CTTCTGATTTTGTAGGTAACCACGTGCGGACCGA

1. A viral particle comprising a nucleic acid construct comprising a transgene encoding a glucocerebrosidase.

2. The viral particle of claim 1, wherein said transgene comprises:
a) a nucleotide sequence selected from the group consisting of SEQ ID NO: 1, 7 11, 12 and 19 or
b) a nucleotide sequence encoding human glucocerebrosidase, wherein human glucocerebrosidase comprises SEQ ID NO: 5, 6, 8, 17 or 18.

3. The viral particle of claim 1 or 2, wherein said nucleic acid construct further comprises a promoter operably-linked to said transgene and wherein said promoter preferably allows the expression of said transgene at least in neuronal and microglial cells of the substantia nigra pars compacta (SNc).

4. The viral particle of claim 3, wherein said promoter is an ubiquitous promoter, notably a promoter selected from the group consisting of GusB promoter comprising or consisting of SEQ ID NO:2 or 20, CAG promoter comprising or consisting of SEQ ID NO:9 or 21, the JeT promoter comprising or consisting of SEQ ID NO: 27 and hSyn promoter comprising or consisting of SEQ ID NO: 13.

5. The viral particle of any one of claims 1-4, wherein said viral particle simultaneously targets at least neurons and microglial cells.

6. The viral particle of any one of claims 1-5, wherein said viral particle simultaneously targets at least dopaminergic neurons and microglial cells in the substantia nigra pars compacta.

7. The viral particle of any one of claims 1-6, wherein said viral particle is a recombinant Adeno-Associated Virus (rAAV) particle, preferably including capsid proteins selected from the group consisting of AAV2, AAV5, AAV9, AAV-MNM004, AAV-MNM008 and AAV TT.

8. The viral particle of any one of claims 1-7, wherein said viral particle comprises AAV TT capsid proteins, which preferably comprises amino acid sequence of SEQ ID NO: 14 or sequence having at least 98.5%, preferably 99% or 99.5% identity with SEQ ID NO: 14.

9. The viral particle of any one of claims 1 to 8, wherein said nucleic acid construct further comprises a polyadenylation signal sequence, preferably a polyadenylation signal sequence comprising SEQ ID NO: 28 or 3, more preferably SEQ ID NO:28.

10. The viral particle of any one of claims 1 to 9, wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype which comprise or consist of sequence SEQ ID NO: 15 and/or 16 or a sequence having at least 80% or at least 90% of identity with SEQ ID NO: 15 and/or 16.

11. The viral particle according to any one of claims 1 to 10, wherein said viral vector comprises SEQ ID NO: 4 or a nucleic acid sequence having at least 80% or at least 90% of identity with SEQ ID NO: 4.

12. The viral particle according to any one of claims 1 to 11, wherein said nucleic acid construct comprises a coding sequence of human glucocerebrosidase under the control of a promoter, allowing expression of said human glucocerebrosidase in at least both dopaminergic neurons and microglial cells, wherein said viral particle is selected among viral particles that targets at least dopaminergic neurons and microglial cells of the substantia nigra pars compacta, typically AAV particles including capsid proteins selected from the group consisting of AAV2, AAV5, AAV9.

13. The viral particle according to any one of claims 1 to 11, wherein said nucleic acid construct comprises a coding sequence of human glucocerebrosidase under the control of a promoter, allowing expression of said human glucocerebrosidase in at least both dopaminergic neurons and microglial cells, preferably in at least neurons of the substantia nigra pars compacta and the cerebral cortex wherein said viral particle is selected among viral particles with retrograde transport, typically viral particles including AAV retro capsid proteins selected from the group consisting of AAV-MNM004, AAV-MNM008 and AAV-TT.

14. The viral particle according to any one of claim 1 to 11 or 13 comprising a nucleic acid construct comprising:
a) a transgene encoding a human glucocerebrosidase; wherein said transgene comprises SEQ ID NO: 19 or a sequence encoding human glucocerebrosidase, wherein human glucocerebrosidase comprises SEQ ID NO: 5 or 8;
b) a promoter operably-linked to said transgene and wherein said promoter preferably allows the expression of said transgene at least in neuronal and microglial cells of the substantia nigra pars compacta (SNc); wherein said promoter is preferably CAG promoter comprising or consisting of SEQ ID NO: 9 or 21 or GusB promoter comprising or consisting of SEQ ID NO: 2 or 20, or the JeT promoter comprising or consisting of SEQ ID NO: 27 or hSyn promoter comprising or consisting of SEQ ID NO: 13;
c) a polyadenylation signal sequence, preferably a polyadenylation signal sequence comprising SEQ ID NO: 28 or 3, preferably SEQ ID NO: 28;
wherein said viral particle is a recombinant Adeno-Associated Virus (rAAV) particle, preferably including capsid proteins of AAV TT, and more preferably comprising SEQ ID NO: 14 or a sequence having at least 98.5%, preferably 99% or 99.5% identity with SEQ ID NO: 14; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype, and wherein each of the 5′ITR and a 3′ITR sequences, independently, comprise or consist of sequences SEQ ID NO: 15 or 16 or a sequence having at least 80% or at least 90% of identity with SEQ ID NO: 15 and/or 16, wherein preferably 5′ITR comprises SEQ ID NO: 15 and 3′ITR comprises SEQ ID NO: 16.

15. The viral particle of any one of claims 1 to 14, wherein said viral particle comprises capsid proteins capable of retrograde transport (AAVretro).

16. The viral particle of claim 15, wherein said viral particle is capable of disseminating in the cerebral cortex, preferably at least to the substantia nigra pars compacta and the cerebral cortex after parenchymal injection in the caudate or putamen nuclei of a non-human primate as determined in an in vivo dissemination assay.

17. An in vivo dissemination assay comprising the steps:
a) injecting into the post-commissural putamen of a non-human primate a rAAV comprising a transgene encoding a green fluorescent protein (GFP) by intraparenchymal injection, and,
b) counting the number of neurons in the cerebral cortex, preferably in the brain areas innervating the caudate putamen nuclei expressing GFP about one month post injection.

18. The in vivo dissemination assay according to claim 17 further comprises a step c) of comparing the number of neurons expressing GFP in the cerebral cortex, preferably in the brain areas innervating the caudate putamen nuclei with a control experiment performed by injecting into the post-commissural putamen of a non-human primate an AAV-TT comprising a transgene encoding a green fluorescent protein (GFP) by intraparenchymal injection.

19. The viral particle of any one of claims 1 to 16 selected among AAVretro which are capable to disseminating in the cerebral cortex, preferably to at least to the substantia nigra pars compacta and the cerebral cortex to at least the same level as AAV-TT as determined in an in vivo dissemination assay according to any of claims 17 to 18.

20. The viral particle according to any one of claims 1 to 16 and 19, wherein said AAVretro is selected from the group consisting of AAV-MNM004, AAV-MNM008 and AAV-TT.

21. The viral particle according to any one of claims 1 to 16 or 19 to 20 wherein said viral particle comprises AAV TT capsid protein, which preferably comprises amino acid sequence of SEQ ID NO: 14 or sequence having at least 98.5%, preferably 99% or 99.5% of identity with SEQ ID NO: 14.

22. The viral particle according to any one of claims 1 to 16 or 19 to 21 for use in therapy.

23. The viral particle according to any one of claims 1 to 16 or 19 to 21 for use in treating synucleinopathy by gene therapy in a subject in need thereof.

24. The viral particle for use according to claim 23, wherein said synucleinopathy is a human sporadic synucleinopathy.

25. The viral particle for use according to claim 23 or 24, wherein said synucleinopathy is a Parkinson's disease, typically sporadic Parkinson's disease.

26. The viral particle for use according to any one of claims 23-25, wherein said subject to be treated is selected among patients with advanced stages of synucleinopathy, typically, at least H-Y stage 3 of Parkinson disease.

27. The viral particle for use according to any one of claims 23-26, wherein said synucleinopathy is not associated to at least a mutation in a gene selected from the group consisting of LRRK2, SNCA, VPS35, GCH1, ATXN2, DNAJC13, TMEM230, GIGYF2, HTRA2, RIC3, EIF4G1, UCHL1, CHCHD2, GBA1, PRKN, PINK1, DJ1, ATP13A2, PLA2G6, FBXO7, DNAJC6, SYNJ1, SPG11, VPS13C, PODXL, PTRHD1, RAB39B, DNAJC13, TMEM230, GIGYF2, HTRA2, RIC3, EIF4G1, UCHL1, and CHCHD2.

28. The viral particle for use according to any one of claims 23-27, wherein said viral particle is administered to said subject by intraparenchymal administration, preferably to the brain area of the substantia nigra pars compacta and/or the caudate putamen nuclei.

29. The viral particle according to any one of claims 1 to 16 or 19 to 21 for use in treating neuronopathic Gaucher's disease.

30. A method for treating a synucleinopathy, preferably Parkinson's disease, and more specifically sporadic Parkinson's Disease, in a subject in need thereof, said method comprising administering to said subject a therapeutically effective amount of a viral particle according to any one of claims 1 to 16 or 19 to 21.

31. The method for treating a synucleinopathy according to claim 30 wherein said subject to be treated is selected among patients with advanced stages of synucleinopathy, typically, at least H-Y stage 3 of Parkinson disease.

32. The method for treating synucleinopathy according to claim 30 or 31 wherein said synucleinopathy is not associated to at least a mutation in a gene selected from the group consisting of LRRK2, SNCA, VPS35, GCH1, ATXN2, DNAJC13, TMEM230, GIGYF2, HTRA2, RIC3, EIF4G1, UCHL1, CHCHD2, GBA1, PRKN, PINK1, DJ1, ATP13A2, PLA2G6, FBXO7, DNAJC6, SYNJ1, SPG11, VPS13C, PODXL, PTRHD1, RAB39B, DNAJC13, TMEM230, GIGYF2, HTRA2, RIC3, EIF4G1, UCHL1, and CHCHD2.

33. The method for treating synucleinopathy according to claims 30-32 wherein said viral particle is administered to said subject by intraparenchymal administration, preferably to the brain area of the substantia nigra pars compacta and/or the caudate putamen nuclei.

34. A method for treating neuronopathic Gaucher's disease, in a subject in need thereof, said method comprising administering to said subject a therapeutically effective amount of a viral particle according to any one of claims 1 to 16 or 19 to 21.
